(19) United States
(12) Patent Application Publication
(10) Pub. No.: US 2025/0090510 A1
(43) Pub. Date: Mar. 20, 2025

(54) eIF4G1-eIF1 INHIBITORS AND USE THEREOF

(71) Applicant: YEDA RESEARCH AND DEVELOPMENT CO. LTD., Rehovot (IL)

(72) Inventors: Rivka DIKSTEIN, Rehovot (IL); Urmila SERAWAT, Rehovot (IL); Ana TAMARKIN BEN-HARUSH, Rehovot (IL); Ora HAIMOV, Rehovot (IL); Neta REGEV-RUDZKI, Rehovot (IL); Shaked ASHKENAZI, Rehovot (IL); Benjamin WEISS, Rehovot (IL); Anastasia LEV, Rehovot (IL)

(21) Appl. No.: 18/964,396

(22) Filed: Nov. 30, 2024

Related U.S. Application Data
(63) Continuation of application No. PCT/IL2023/050571, filed on Jun. 1, 2023.

(30) Foreign Application Priority Data
Jun. 1, 2022 (IL) 293532

(51) Int. Cl. A61K 31/44; A61K 31/415; A61K 31/428

(52) U.S. Cl. CPC A61K 31/44; A61K 31/415; A61K 31/428

(57) ABSTRACT

Methods of inhibiting eIF4G1 binding to eIF1, and inhibiting translation initiation are provided. Pharmaceutical compositions comprising inhibitors of eIF4G1-eIF1 binding and there use in treating disease are also provided.

### CROSS REFERENCE TO RELATED APPLICATIONS

This application is a ByPass Continuation of PCT Patent Application No. PCT/IL2023/050571 having International filing date of Jun. 1, 2023, which claims the benefit of priority of Israeli Patent Application No. 293532, filed Jun. 1, 2022 titled “eIF4G1-eIF1 INHIBITORS AND USE THEREOF”. The contents of which are all incorporated herein by reference in their entirety.

### REFERENCE TO AN ELECTRONIC SEQUENCE LISTING

The contents of the electronic sequence listing (YEDA-P-015-PCT.xml; Size: 61,710 bytes; and Date of Creation: May 23, 2023) is herein incorporated by reference in its entirety.

### FIELD OF INVENTION

The present invention is in the field of translation inhibition.

### BACKGROUND OF THE INVENTION

Protein synthesis is the foundation of cellular functioning. The most tightly regulated stage in this process is translation initiation, whereby the ribosomal subunits, eukaryotic initiation factors (eIFs), and other components assemble at the initiation codon of mRNA. In eukaryotes, the majority of the mRNAs initiate translation through a canonical mode involving several steps (i) recognition of the mRNA 5′end m7G cap structure; (ii) assembly of the 43S Pre-Initiation Complex (PIC); (iii) recruitment of the PIC to the mRNA (iv); scanning of the 5′UTR; (v) start codon selection; and (vi) 60S subunit joining. Flexibility in the regulation of protein synthesis allows cells to rapidly adapt to stress, activate survival programs, and develop drug resistance. Therefore, aberrant control of mRNA translation is universal in cancer, and several common anti-cancer drugs act, at least in part, by inhibiting translation. Dysregulated translation is also involved in the pathophysiology of numerous neurodegenerative diseases and brain-associated disorders. Thus, inhibitors of translation initiation hold promise as versatile therapeutic agents.

The key factor that mediates m7G cap recognition is eIF4F, a complex consisting of eIF4E, the cap-binding protein; eIF4G1, a large scaffolding protein that interacts with eIF4E and recruits the 43S; and the helicase eIF4A, which unwinds cap-proximal secondary structures. The 43S PIC recruitment to the mRNA is mediated by direct interaction between eIF4G1 and ribosome-bound eIF3 and eIF1. The critical scanning phase is promoted by eIF1 and eIF1A, which bind the 40S subunit near the P and A sites, respectively, and promote an open 40S conformation that is scanning competent. A recent study, by the Inventors, from human cells revealed that eIF4G1 interaction with eIF1 is dynamic and is required to promote scanning and leaky scanning from cap-proximal AUG. However, the regulatory role of this interaction in the translation of endogenous mammalian mRNAs is presently unknown.

In recent years, substantial progress has been made in mapping the location and the interactions of the various ribosomal proteins (RPs) and eIFs of the initiation complex by structural cryo-EM and biochemical studies. A Cryo-EM structure of the 48S complex captured in the process of scanning and included eIF1 and a core segment eIF4G1 was recently reported. However, the interactions between eIF4G1 and eIF1 and the interactions of eF4G1 with several eIF3 subunits were not visible, raising the possibility that an alternative conformation of a scanning complex may exist whereby eIF4G1 directly interacts with eIF1. Additionally, much less is known about the functional significance and the regulation of these dynamic protein-protein interactions (PPIs) during the multi-step translation initiation process, especially in the context of mammalian cells. As mRNA translation is critical for all cellular activities, most of the eIFs and RPs are essential for cell growth and viability, rendering the use of genetic manipulation impractical. This poses a major challenge for elucidating the in-vivo significance of the various steps of the initiation process and the specific roles played by each initiation factor in mammalian cells. Presently the investigation of eIFs utilizes prolonged treatments with siRNAs, which impede the ability to distinguish between direct and indirect effects, and is also expensive and labor-intensive. Likewise, overexpression studies give rise to non-physiological amounts of proteins, limiting our ability to draw conclusions. Furthermore, some eIFs, such as eIF4G1, eIF1A, and eIF2β, harbor several independent, sometimes opposing, functional domains making the information gleaned from their overall depletion less informative. Thus, there is a significant interest in finding alternative avenues for addressing the significance of eIFs for translation in-vivo.

While the importance of protein-protein interactions (PPI) for translation initiation is well-established, their targeting by drugs is rare, primarily since PPI involves multiple contact sites, and finding small molecules that can effectively disrupt these contacts is challenging. New small molecule inhibitors of translation initiation and in particular eIF4G1 interaction with eIF1 are therefore greatly needed.

### SUMMARY OF THE INVENTION

The present invention provides methods of inhibiting eIF4G1 binding to eIF1, and inhibiting translation initiation. Pharmaceutical compositions comprising inhibitors of eIF4G1-eIF1 binding and there use in treating disease are also provided.

According to a first aspect, there is provided a method of inhibiting Eukaryotic translation initiation factor 4 gamma 1 (eIF4G1) binding to Eukaryotic translation initiation factor 1 (eIF1) comprising contacting the eIF4G1 with a compound, a salt thereof, a tautomer thereof, a functional derivative thereof or any combination thereof; wherein the compound is represented by Formula I:

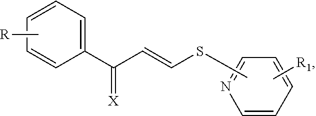

### or by Formula II:

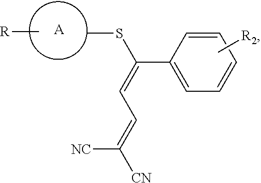

wherein:
each R and R₂ is independently selected from H, or is absent, or represents a substituent comprising any one of —NO₂, —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted;
each R′ is independently H or comprises an optionally substituted C₁-C₁₀ alkyl, an C₁-C₁₀ alkyl-aryl, an C₁-C₁₀ alkyl-cycloalkyl, optionally substituted C₃-C₁₀ cycloalkyl, optionally substituted C₃-C₁₀ heterocyclyl, optionally substituted heteroaryl, optionally substituted aryl or a combination thereof;
A represents any of cycloalkyl, aryl, and heteroaryl, a fused aryl, a fused cycloalkyl or any combination thereof;
X comprises O, S, NH, or NR′;
R₁ is H or represents a substituent comprising any one of an electron withdrawing group; —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted.

According to some embodiments, the R₁ is the electron withdrawing group.

According to some embodiments, the X is O.

According to some embodiments, the A is heteroaryl, and wherein the R₂ is not H.

According to some embodiments, the compound is selected from:

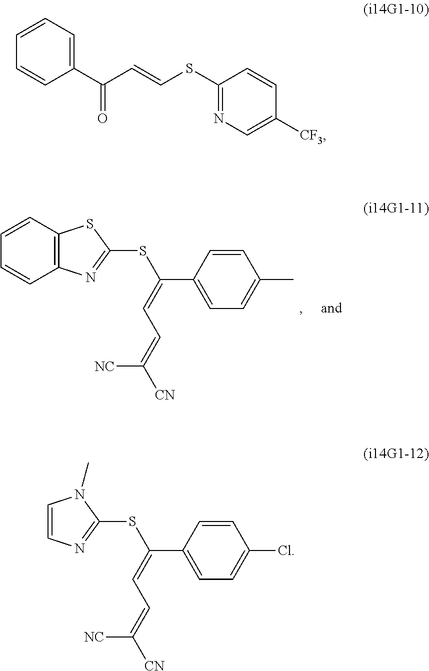 [SMILES: O=C(/C=C/Sc1ccc(C(F)(F)F)cn1)c1ccccc1 ; Cc1ccc(/C(=C\C=C(C#N)C#N)Sc2nc3ccccc3s2)cc1 ; Cn1ccnc1S/C(=C/C=C(C#N)C#N)c1ccc(Cl)cc1]

According to some embodiments, the eIF4G1 and the eIF1 are in a cell and the method is a method of decreasing translation initiation in the cell.

According to some embodiments, the contacting is with i14G1-10 or a derivate thereof and wherein the contacting further increases binding of the eIF4G1 to Eukaryotic translation initiation factor 4E (eIF4E).

According to some embodiments, the method is a method of increasing translation from open reading frames with short 5′ UTRs and decreasing translation from open reading frames with long 5′ UTRs.

According to some embodiments, a short 5′ UTR is a UTR of at most 150 nucleotides and a long 5′ UTR is a UTR of greater than 150 nucleotides.

According to some embodiments, the method is a method of increasing recognition of non-AUG translational start codons.

According to some embodiments, the non-AUG codons are selected from ACG, CUG, GUG and UUG.

According to some embodiments, the method is a method of increasing recognition of a most cap-proximal AUG codon and decreasing leaky recognition of more downstream AUG codons.

According to some embodiments, the method is a method of increasing translation of at least one stress-response protein.

According to some embodiments, the stress-response protein is selected from an unfolded protein response (UPR) pathway protein, an endoplasmic reticulum (ER)-stress response pathway protein and a UV-response pathway protein.

According to some embodiments, the stress-response protein is selected from Activating transcription factor 3 (ATF3), Activating transcription factor 4 (ATF4), Growth arrest and DNA damage inducible alpha (GADD45A), DNA damage inducible transcript 3 (DDIT3) and Protein phosphatase 1 regulatory subunit 15A (PPP1R15A or GADD34).

According to some embodiments, the method is a method of killing the cell.

According to some embodiments, the cell is characterized by increased protein expression or increased number of upstream open reading frames (uORFs) as compared to a healthy control cell.

According to some embodiments, the contacting is with either i14G1-10, i14G1-12 or a combination thereof.

According to another aspect, there is provided a method of reducing translation in a target cell, the method comprising reducing binding of eIF4G1 to eIF1 without increasing binding of eIF4G1 to eIF4E in the cell, thereby reducing translation in a target cell.

According to some embodiments, the method comprises contacting the cell with a compound that binds eIF4G1 at an eIF1 binding site and occludes, blocks or otherwise makes inaccessible an eIF4E binding site in eIF4G1.

According to some embodiments, the compound is represented by Formula II or a salt, tautomer, or functional derivative thereof.

According to some embodiments, the agent is i14G1-11, i14G-12 or a functional derivative thereof.

According to another aspect, there is provided a pharmaceutical composition comprising a compound, a salt thereof, a tautomer thereof, a functional derivative thereof or any combination thereof; wherein the compound is represented by Formula I:

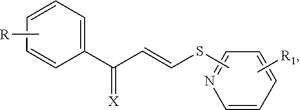

### or by Formula II:

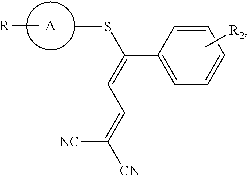

wherein:
each R and R₂ is independently selected from H, or is absent, or represents a substituent comprising any one of —NO₂, —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted;
each R′ is independently H or comprises an optionally substituted C₁-C₁₀ alkyl, an C₁-C₁₀ alkyl-aryl, an C₁-C₁₀ alkyl-cycloalkyl, optionally substituted C₃-C₁₀ cycloalkyl, optionally substituted C₃-C₁₀ heterocyclyl, optionally substituted heteroaryl, optionally substituted aryl or a combination thereof;
A represents any of cycloalkyl, aryl, and heteroaryl, a fused aryl, a fused cycloalkyl or any combination thereof;
X comprises O, S, NH, or NR′;
R₁ is H or represents a substituent comprising any one of an electron withdrawing group; —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted.
and pharmaceutically acceptable carrier, excipient or adjuvant.

According to some embodiments, the R₁ is the electron withdrawing group.

According to some embodiments, the X is O.

According to some embodiments, the A is heteroaryl, and wherein the R₂ is not H.

According to some embodiments, functional is functional in inhibiting interaction of eiF4G1 and eIF1.

According to some embodiments, the pharmaceutical composition is formulated for administration to a subject.

According to some embodiments, the pharmaceutical composition is for use in a method of the invention.

According to another aspect, there is provided a method of treating a disease, disorder or condition in a subject in need thereof, wherein the disease or condition is treatable by inhibiting translation, the method comprising administering to the subject a pharmaceutical composition of the invention, thereby treating a disease, disorder or condition.

According to some embodiments, the disease is characterized by aberrant translation.

According to some embodiments, the disease is selected from cancer, a thrombotic disorder, a parasitic infection and a neurodegenerative disease.

According to some embodiments, the disease is cancer.

According to some embodiments, the cancer is selected from breast cancer, lung cancer, colorectal cancer, ovarian cancer and bone cancer.

According to some embodiments, the method is for treating a malaria infection.

According to some embodiments, the malaria infection is caused by P. falciparum infection.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-G: Identification and initial characterization of eIF1-eIF4G1 inhibitors using a high-throughput drug screen (HTS). (1A) A flowchart describing the steps of the recombinant eIF1-eIF4G1 split-RL HTS. (1B) Selection of drugs that inhibit leaky scanning. HEK293T cells were transfected with a short 5′UTR bearing GFP reporter gene described on the top, treated with the indicated HTS hits, and then analyzed by western blot with GFP antibody. US and DS denote upstream and downstream AUG initiation sites, respectively. (1C) A graph showing the change in the ratio of the upstream and downstream AUG following treatment with the indicated drugs. (1D) The chemical structure of the eIF1-eIF4G1 inhibitors, i14G1-10 and i14G1-12. (1E) His-tagged eIF4G1 and His-tagged eIF1 were expressed in BL21 bacteria and purified using nickel NTA beads. Purified His-tagged eIF4G1 and His-tagged eIF1 were incubated with the indicated doses of either i14G1-10 (0-256 μM) or i14G1-12 (0-256 μM) for 5 minutes, followed by fluorescence measurement. For eIF4G1 excitation was at 280 nm and emission was at 350 nm. For eIF1 excitation was at 274 nm and emission was at 304 nm. The data shown represent three independent replicates. IC50 was calculated using Graphpad prism 9. (1F-1G) Western blots of GST pull down. GST-eIF4G1 was coupled to glutathione Sepharose beads and incubated with recombinant His-eIF1 in the absence or presence of the indicated concentrations of (1F) i14G1-10 and (1G) i14G1-12. The pulled-down complexes were washed and run on SDS-PAGE followed by western blot with anti-6×His antibody. The GST-eIF4G1 used for the binding reactions are shown in the Coomassie blue stained gel shown on the bottom of each panel.

FIGS. 2A-C: The effect of i14G1-10 and i14G1-12 on eIF1-eIF4G1 and eIF4E-eIF4G1 complexes. (2A-B) HA-eIF1 and HA-eIF4E were each transfected into HEK293T cells and 48 h after transfection cells were treated with DMSO or i14G1-10 (2A) or i14G1-12 (2B) for 4 h, followed by immunoprecipitation of HA-eIF1 or HA-eIF4E using anti-HA-agarose beads. Western blot was run to check co-immunoprecipitation of endogenous eIF4G1 with HA-eIF1 or HA-eIF4E. The graphs represent the co-immunoprecipitation of eIF4G1 with HA-eIF1 (right) or HA-eIF4E (left). Co—IP data shown as mean±SEM of 4-7 independent experiments. The asterisks denote statistically significant difference compared to DMSO, according to Student's t-tests (one-tailed, paired). *p<0.05. (2C) HEK293T cells were treated with DMSO, i14G1-10, or i14G1-12 for 4 h, followed by cell lysis. Respective cell lysates were incubated with cap analog (γ-Aminohexyl-m7GTP) beads for 2 h to precipitate bound eIF4E. Western blot was performed to check co-precipitation of eIF4G1 along with eIF4E on the cap-analog beads. The graph represents the binding fraction of eIF4G1 and eIF4E to the cap-analog beads (mean±SEM) with DMSO (black), i14G1-10 (blue), and i14G1-12 (red), n=6. The asterisks denote statistically significant difference compared to DMSO, according to Student's t-tests (one-tailed, paired). *p<0.05; **p<0.01.

FIGS. 3A-F: i14G1-10 and i14G1-12 inhibit mRNA translation and sensitize cell survival of eIF4G1 and eIF1 depleted cells. (3A) In-vitro translation using rabbit reticulocyte lysate which was pre-incubated with different concentrations of i14G1-10 or i14G1-12 for 10 minutes followed by the addition of capped firefly luciferase mRNA. After 90 minutes at 30° C., the luminescence signal was measured and the IC50 was calculated. The data represents mean±SEM of 6 independent repeats. (3B) HEK293T cells were treated with DMSO (vehicle control), and sub-cytotoxic concentrations of i14G1-10 (20 μM) and i14G1-12 (10 μM) for 3h. Cell lysates were then subjected to sucrose gradient sedimentation to obtain polysome profiles. (3C) HEK293T cells were treated with the indicated concentrations of i14G1-10 and i14G-12 for 3 h, followed by the addition of puromycin (10 μg/ml) for 5 minutes, after which cells were lysed and analyzed by western blot using an anti-puromycin antibody and anti-GAPDH antibody that serves as a loading control. The graph represents the mean±SEM chemiluminescence signal intensity of puromycin labeling normalized to GAPDH of 3 independent experiments. The calculated IC-50 is shown. (3D) HEK293T cells were transfected with either control or eIF4G1 siRNA in the absence or presence of eIF4G1 expression plasmid. As the eIF4G1 siRNA pool is directed against the 3′UTR, which is absent from the eIF4G1 expression plasmid, the exogenous eIF4G1 is resistant to the siRNA. 48 h after transfection, cells were treated with the indicated concentrations of i14G1-10 and i14G-12 for 3 h, followed by a 5 minutes puromycin pulse as described in 3C. The graph represents the mean±SEM chemiluminescence signal intensity of puromycin labeling normalized to GAPDH of 3 independent experiments. The calculated IC-50 is shown. (3E) The graphs represent cell viability of the indicated cell lines in the presence of increasing concentrations of i14G1-10 and i14G1, analyzed by Luminescent Cell Viability Assay. (3F) Pie charts showing the cell cycle distribution of HEK293T cells treated with DMSO, i14G1-10 and i14G1-12.

FIGS. 4A-L: The effect eIF1-eIF4G1 inhibition by i14G1-10 or i14G1-12 on global translation is linked to start codon stringency. (4A) HEK293T cells were treated with DMSO, i14G1-10 (20 μM), and i14G-20 (10 μM) for 3 h and then ribosome foot printing libraries were prepared, sequenced and analyzed as described in the scheme. The presented graphs show meta-gene analysis of the distribution of normalized reads in the coding region (CDS) and 5′UTR of all analyzed genes in DMSO (black), i14G1-10 (blue), and i14G1-12 (red). (4B) Summary of the number of genes whose ribosomal occupancy was affected (fold change, ≥1.7 or 1.7) or unaffected in coding regions (CDS) (left) and 5′UTR (right) in i14G1-10 or i14G1-12 samples. (4C-D) Venn diagram presenting the overlap between the CDS upregulated and CDS downregulated genes with the 5′ UTR upregulated genes following (4C) i14G1-10 or (4D) i14G1-12 treatments. (4E) Analysis of the nucleotide context of the annotated start codon of i14G1-10 and i14G1-12 differentially translated mRNAs using the STREME motif discovery algorithm. (4F) The translation efficiency of eIF1 in DMSO, i14G1-10 and i14G1-12 samples calculated from the Ribo-seq data. The weak AUG context of eIF1 is shown above. (4G) Analysis of the nucleotide context of the 5′ UTR translation initiation site (TIS) in DMSO and the upregulated gene sets of i14G1-10 and i14G1-12 treatments, respectively using the MEME program. (4H) A pie chart showing the frequency of the start codon in the ribosome footprints in the 5′ UTR of DMSO and the upregulated gene sets of i14G1-10 and i14G1-12. (4I) HEK293T cells were transfected with a firefly luciferase reporter gene driven by either by AUG or near cognate start codon, i.e., ACG, CUG, GUG, and UUG. The Renilla luciferase reporter gene was also co-transfected and served as a normalizing control. The near cognate start codon activities in DMSO control and i14G1-10 and i14G1-12 treated cells are presented as a percentage of AUG activity. The data are shown as mean±SEM, n=5. the asterisks denote statistically significant difference compared to DMSO, according to Student's t-tests (one-tailed, paired). *p<0.05; **p<0.01. (4J) Scatter plots showing the translation efficiencies correlations (R2 >0.9) of all analyzed genes between the two independent replicate experiments with DMSO (vehicle control) (top), i14G1-10 (middle), and i14G1-12 (bottom). (4K) Scatter plots showing the translation efficiencies correlations between 5′UTR and CDS (Pearson's r >0.5) of all analyzed genes of two independent replicate experiments with i14G1-10 (left) and i14G1-12 (right). (4L) Venn diagrams showing the overlaps of 5′UTR upregulated genes (left) and CDS downregulated genes (center), and CDS upregulated (right) between i14G1-10 and i14G1-12 treatments.

FIGS. 5A-D: 5′UTR length-dependent translational control by the eIF1-eIF4G1. (5A) Box plots showing the relationship between the 5′ UTR length and the translation efficiency of genes whose translation efficiency of the CDS was unaffected, downregulated, and upregulated by i14G1-10 or i14G1-12 treatments. The asterisks denote a statistically significant difference. **p<0.01, ****p<0.0001. (5B) A scheme of the firefly reporter genes that are driven by long (354 nt) and short (111 nt) 5′ UTR length, is shown on the top. HEK293T cells were transfected with these reporter genes. After 4 h, transfected cells were treated with DMSO, i14G1-10 (5 μM), or i14G1-12 (5 μM) for 16 h and analyzed for firefly luciferase activities. The graph presents the normalized luminescence activity of the long 5′UTR and the short 5′UTR reporters in the indicated treatments. The data are shown as mean±SEM, n=6. The asterisks denote statistically significant difference compared to DMSO, according to Student's t-tests (one-tailed, paired). *p<0.05; **p<0.01. (5C-D) HEK293T cells were treated with DMSO, i14G1-10, and i14G1-12 for 3 h, lysed, and subjected to sucrose gradient sedimentation and fractionation. Fractions were pooled according to polysome profile as free (grey), light (blue), and heavy (yellow) polysomal fractions. cDNA was prepared from each fraction pool and real-time PCR was performed for the indicated TISU genes (5C) or histone genes (5D). The data are shown as mean±SEM, n=3. The asterisks denote statistically significant difference compared to DMSO, according to Student's t-tests (one-tailed, paired), *p<0.05; **p<0.01.

FIGS. 6A-G: i14G1-10 and i14G1-12 uncover roles of the eIF4G1-eIF1 complex in ISR, cell cycle and cell survival. (6A) The top enriched biological categories of the i14G1-10 and i14G1-12 translationally upregulated genes along with their P-values. (6B). Ribo-seq and TI-seq read tracks of representative stress response-associated mRNAs (i.e., ATF3, and GADD45A) in DMSO (black), i14G1-10 (red), and i14G1-12 (green) samples. The red triangle represents 5′UTR TIS codons and the green denotes annotated TIS. Black arrows show the translation direction. (6C) Mouse embryonic fibroblasts (MEFs) control cells and eIF2αS52A mutant MEF cells were treated with Thapsigargin (1 μM) for 1h. Cells were lysed, and cell lysate was analyzed using western blot with the indicated antibodies. (6D) Control cells and eIF2αS52A mutant MEF cells were treated with DMSO, i14G1-10 (30 μM) or i14G1-12 (15 μM) for 3 h, cells were lysed in polysome buffer and loaded onto sucrose density gradient for polysome separation followed by fractionation. The graph presents the polysomes vs monosomes ratio in control MEFs (purple) and eIF2αS52A mutant cells (blue). (6E) Fractions were pooled (from 6D) according to the polysome profile as free (grey), light (blue), and heavy (yellow) polysomal fractions. cDNA was prepared from each fraction pool and real-time PCR was performed for the indicated stress response genes. The data are shown as mean±SEM, n=3. The asterisks denote statistically significant differences compared to DMSO, according to Student's t-tests (one-tailed, paired), *p<0.05; **p<0.01. (6F-G). HEK293T cells were transfected with HA-eIF1 or HA-eIF4E, 48 h later, treated with (6F) 1 μM Thapsigargin or (6G) 250 nM Torin-1. Cell lysates were subjected to immunoprecipitation with HA-agarose beads to precipitate HA-eIF1 and HA-eIF4E to check for co-IP of eIF4G1 using western blot (left). The graphs on the right represent the relative chemiluminescence signal intensity of the co-immunoprecipitation of eIF4G1 with HA-eIF1 or HA-eIF4E of 4 independent experiments. The asterisks denote statistically significant difference compared to DMSO *p<0.05; **p<0.01.

FIGS. 7A-E: (7A) Ribo-seq and TI-seq read tracks of representative stress response-associated mRNAs (DDIT3 and GADD34) in DMSO (black), i14G1-10 (red), and i14G1-12 (green) samples. The red triangle represents 5′UTR TIS codons and the green denotes annotated TIS. Black arrows show the translation direction. (7B) The upper panel shows the Ribo-seq and TI-seq read tracks of ATF4 gene in DMSO (black), i14G1-10 (red), and i14G1-12 (blue). The graphs in the lower panel are the quantification of ATF4's uORF1′, uORF1, uORF2, and main-ORF reads of the Ribo-seq data. The asterisks denote statistically significant difference compared to DMSO, according to Student's t-tests (one-tailed, paired). **p<0.01, ***p<0.001, ****p<0.0001. (7C) HEK293T cells were treated with DMSO, i14G1-10, and i14G1-12 for 3 h, lysed and subjected to sucrose gradient sedimentation and fractionation. Fractions of the free (grey), light (blue), and heavy (yellow) were pooled. cDNA was prepared from each fraction pool and real-time PCR was performed for the indicated ISR genes. The data are shown as mean±SEM, n=3. The asterisks denote statistically significant difference compared to DMSO, according to Student's t-tests (one-tailed, paired). *p<0.05; **p<0.01. (7D) HEK293T cells were treated with DMSO, i14G1-10, and i14G1-12 for 3 h. The respective cell lysates were subjected to western blot to check the protein levels of ATF3 and ATF4 (left). The graph represents the relative chemiluminescence signal intensity (mean±SEM) for ATF4 and AT3 normalized to GAPDH protein levels of three biological replicates. The asterisks denote statistically significant difference compared to DMSO, according to Student's t-tests (one-tailed, paired), *p<0.05. (7E) Control MEFs and eIF2α S52A mutant MEFs were treated with DMSO (vehicle control), and sub-cytotoxic concentrations of i14G1-10 (30 μM) and i14G1-12 (15 μM) for 3h. Cell lysates were then subjected to sucrose gradient sedimentation to obtain polysome profiles.

FIGS. 8A-B: (8A) Bar graph of percent parasitemia in blood samples treated with i14G1-10 (20 μM) and i14G1-12 (10 μM) for 1 hour. DMSO treatment was used as a negative control. *=pVal; <0.05, ***=pVal<0.001 (8B) Photograph of a western blot and corresponding gel total protein staining of P. falciparum blood cultures treated with the inhibitors with or without the addition of puromycin. DMSO treatment and untreated cultures were used as a negative control.

FIGS. 9A-H: Inhibition of eIF1-eIF4G1 leads to 48S instability and scanning defect (9A) Polysome profiles of HEK293T cells treated with DMSO or i14G1-12 after crosslinking with formaldehyde, lysis and Rnase I digestion. The grey layer represents the fractions corresponding to the 40S ribosome used for the TCP-seq library preparation. (9B) Heatmap representing the log 2 mean of the relative number of reads mapping to the individual tRNAs in i14G1-12 over DMSO (n=3). The number of reads was normalized to the codon frequency in the human transcriptome. (9C) Volcano plot showing log 2 fold change (FC) of the abundance of each tRNAs comparing i14G1-12 over DMSO with FDR <0.05 and a log 2 FC threshold of 1. (9D) Histograms showing the relative number of reads mapping to mRNA in DMSO vs. i14G1-12. (9E) Metagene plots of normalized ribosome footprints (RPFs) counts on leader region. Numbers indicate the slope of the regression lines. (9F) Volcano plot showing log 2 fold change of the differential initiation analysis in i14G1-12 over DMSO. Transcripts showing significantly lower (log 2 FC <−1, FDR <0.05) or higher (log 2 FC >1, FDR 0.05) abundance are represented in red or blue, respectively. (9G) Bar chart of the relative number of reads mapping to each mRNA feature in DMSO and i14G-12. (9H) Reactome terms of the downregulated scanned population in i14G1-12 over DMSO.

FIGS. 10A-J: Definition of leaky scanning and its regulation by specific mRNA features and eIF1-eIF4G1. (10A) Metagene plots of normalized ribosome footprints (RPFs) counts on CDS region. (10B) Scatter plot representing the leaky scanning score (LS) of transcripts in i14G1-12 versus DMSO. Transcripts with a significant LS (FDR <0.05) in DMSO and i14G1-12 are highlighted. (10C) Weblogos representing the motif sequence around the start codon of transcripts that are significantly leaky (left panel, LS >3, FDR <0.05) or non-leaky (right panel, LS<3, FDR <0.05) in DMSO. (10D) Boxplots of the leaky scanning score in DMSO and in i14G1-12. (10E) Weblogos representing the motif sequence around the start codon of transcripts that are significantly leaky (left panel, LS >3, FDR <0.05) or non-leaky (right panel, LS<3, FDR <0.05) in i14G1-12. (10F) Plot of the mean GC content in a 20 nt window around the start codon of leaky and non-leaky transcripts in DMSO and i14G1-12. (10G) Boxplot representing the log 5′ UTR length of leaky and non-leaky transcripts in DMSO and i14G1-12. (10H-10I) Weblogos of the motif sequence around the start codon of transcripts with short 5′ UTR (less than 50 nt) that are significantly leaky or non-leaky in DMSO (10H) and i14G1-12 (10I). (10J) Bar charts of the FDR from selected GO biological process terms that are significantly enriched from the leaky or non-leaky genes in DMSO and in i14G1-12.

FIGS. 11A-H: Characterization of initiation site footprints. (11A) Matrices of the read length versus the distance of 5′ ends (left panel) or 3′ ends (right panel) read positions relative to the start codon in DMSO. The sum of normalized counts per million of the three replicates for each position and length is displayed. (11B) A scheme representing the populations A, B, and C. (11C-11D). Plots of the normalized 5′ read counts (11C) or 3′ read counts (11D) around the start codon in DMSO and i14G1-12. (11E) Ven diagram of the number of genes in population A, B and C. (11F) Histogram of the relative amount of reads from population B and C. (11G) Histogram of the relative amount of reads in population B over A in DMSO and i14G1-12. (11H) Weblogos representing the motif sequence around start codon of transcripts from population A (left panel), B (middle panel) and C (right panel).

FIGS. 12A-E: Initiation site footprints in human mice and yeast. 12A-12D. Plots of the normalized 3′ read counts around the start codon from the TCP-seq data from (12A) HEK 293T, (12B) HeLa and NIH 3T3 cells, (12C) S. cerevisiae and S. pombe and (12D) HeLa and NIH 3T3 treated with harringtonine. (12E) Motif sequence around the start codon of transcripts with (left) or without (right) reads ending at +24-+26 in HEK 293T cells from this study.

FIGS. 13A-F: Characterization of initiation site footprints with Sel-TCP-seq uncovers the rearrangement of the 48S during elongation. 13A-13C. Plots of the normalized 3′ read counts around the start codon from the selective TCP-seq data against (13A) eIF3 in HeLa (left panel) or S. cerevisiae (right panel), (13B) eIF2α, (13C) eIF4E1 and eIF4G1 in HeLa cells. (13D) The ratio of the number of reads ending at +21-+24 over +18 from Sel-TCP-seq in HeLa. (13E) Violin plots showing the lengths of the reads ending at +18 (red violins) or +21-+24 (green violins) from total and Sel-TCP-seq in HeLa. (13F) Schemes representing the different conformations. Numbers indicate the edges of the complex on the mRNA (relative to the start codon).

### DETAILED DESCRIPTION OF THE INVENTION

The present invention, in some embodiments, provides methods of inhibiting eIF4G1 binding to eIF1, and inhibiting translation initiation. Pharmaceutical compositions comprising inhibitors of eIF4G1-eIF1 binding and their use in treating disease are also provided.

The invention is based, at least in part, on the surprising identification and characterization of the first inhibitors against the eIF4G1-eIF1 complex. By applying a functional assay that measures cap-proximal AUG selection, we identified two small molecule inhibitors, i14G1-10 and i14G1-12, that faithfully reproduced the inhibitory effects exerted upon eIF4G1 and eIF1 knockdown and facilitated the discovery of a previously unknown mechanism of translation activation of ER/UPR stress-response genes by eIF4G1-eIF1 that is largely eIF2α phosphorylation independent.

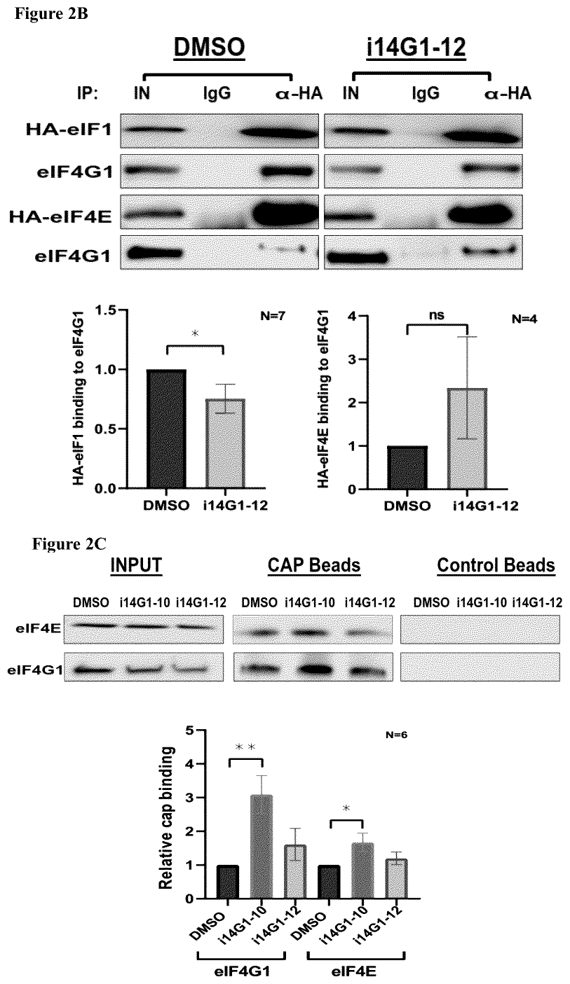

Both i14G1-10 and i14G1-12 bind directly to eIF4G1, but their inhibitory mechanism is somewhat different, as i14G1-10 interferes with the eIF4G1-eIF1 interaction and facilitates the eIF4G1-eIF4E complex formation while i14G1-12 mostly inhibits eIF4G1 binding to eIF1 (FIG. 2A-C). The impact of the two compounds on translation is similar, though i14G1-12 was more potent in vivo.

By a first aspect, there is provided a method of inhibiting Eukaryotic translation initiation factor 4 gamma 1 (eIF4G1) binding to Eukaryotic translation initiation factor 1 (eIF1) comprising contacting the eIF4G1 with a compound or a salt thereof, a tautomer thereof, a functional derivative thereof or any combination thereof.

In some embodiments, the compound is represented by Formula I:

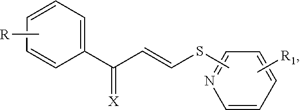

wherein:
R is H, or is absent, or represents a substituent comprising any one of —NO₂, —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted; each R′ is independently H or comprises an optionally substituted C₁-C₁₀ alkyl, an C₁-C₁₀ alkyl-aryl, an C₁-C₁₀ alkyl-cycloalkyl, optionally substituted C₃-C₁₀ cycloalkyl, optionally substituted C₃-C₁₀ heterocyclyl, optionally substituted heteroaryl, optionally substituted aryl or a combination thereof; X comprises O, S, NH, or NR′; R₁ is H or represents a substituent comprising any one of an electron withdrawing group; —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted.

The term “electron-withdrawing group” is well known to those of skill in the art as a functional group that draws electrons to itself more than a hydrogen atom would if it occupied the same position in the molecule, as described in J. March, Advanced Organic Chemistry, third edition, Pub: John Wiley & Sons, Inc (1985).

Exemplary electron-withdrawing group include, but are not limited to, nitro group, fluoro, haloalkyl, halocycloalkyl, haloaryl, halo heteroaryl, a cyano group, an alkyloxy carboxylic ester bond, a sulfonyl group, a sulfonate group, a sulfinyl group, a sulfonamide group, an azo group, a guanidine group, and a carboxylic acid derivative, or any combination thereof. The term “carboxylic acid derivative” as used herein encompasses carboxy, amide, carbonyl, anhydride, carbonate ester, and carbamate.

In some embodiments, electron-withdrawing group encompasses a haloalkyl (e.g., C₁-C₆ or C₁-C₁₀ haloalkyl).

In some embodiments, the compound is represented by Formula IA:

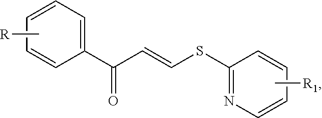

wherein R and R1 are as described herein. In some embodiments, the compound is represented by Formula I or by Formula IA, wherein at least one of R and R1 is not H. In some embodiments, the compound is represented by Formula I or by Formula IA R1 is an electron-withdrawing group.

In some embodiments, the compound is represented by Formula IB:

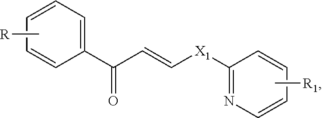

wherein R and R1 are as described herein, and wherein X1 is O, N(R)₁₋₂, or S as allowed by valency.

In some embodiments, the compound is or comprises

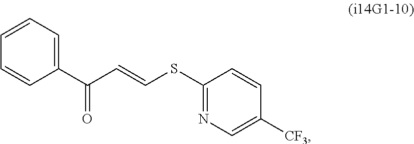 [SMILES: O=C(/C=C/Sc1ccc(C(F)(F)F)cn1)c1ccccc1]

including any tautomer, any salt, or any functional derivative thereof.

In some embodiments, the compound is represented by Formula II:

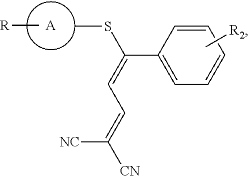

wherein: each R and R₂ is independently selected from independently H, or absent, or represents a substituent comprising any one of —NO₂, —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted; each R′ is independently H or comprises an optionally substituted C₁-C₁₀ alkyl, an C₁-C₁₀ alkyl-aryl, an C₁-C₁₀ alkyl-cycloalkyl, optionally substituted C₃-C₁₀ cycloalkyl, optionally substituted C₃-C₁₀ heterocyclyl, optionally substituted heteroaryl, optionally substituted aryl or a combination thereof; A represents any of cycloalkyl, aryl, heteroaryl, a fused aryl, a fused cycloalkyl or any combination thereof; X comprises O, S, NH, or NR′; R₂ is H or represents a substituent comprising any one of an electron withdrawing group; —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted. In some embodiments, A represents a heteroaryl. Non-limiting examples of heteroaryls include but are not limited to: benzimidazolyl, imidazolyl, pyrrole, furane, thiophene, imidazole, oxazole, thiazole, pyrazole, pyridine, pyrimidine, quinoline, isoquinoline and purine. Additional heteroaryl include: pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydroazepino[3,4-b]indolyl, 1H-2,3,4,5-tetrahydroazepino-[4,3-b]indolyl, 1H-2,3,4,5-tetrahydroazepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl.

In some embodiments, R2 is selected from H, C₁-C₁₀ alkyl, halo, C₁-C₁₀ haloalkyl, optionally substituted C₁-C₁₀ alkyl, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, cycloalkyl, or any combination thereof. In some embodiments, R is selected from H, C₁-C₁₀ alkyl, or is absent.

In some embodiments, the compound is represented by Formula IIA:

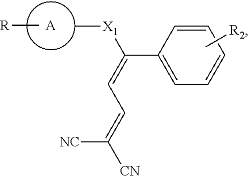

wherein R and R2 are as dilsoced herein, and wherein X1 is O, N(R)₁₋₂, or S as allowed by valency.

In some embodiments, the compound is represented by Formula II or by Formula IIA, wherein at least one of R and R2 is not H. In some embodiments, the compound is represented by Formula II or by Formula IIA, wherein R2 is selected from C₁-C₁₀ alkyl, halo, C₁-C₁₀ haloalkyl, and a substituted C₁-C₁₀ alkyl. In some embodiments, the compound is represented by Formula II or by Formula IIA, wherein R2 is selected from C₁-C₁₀ alkyl, halo, C₁-C₁₀ haloalkyl, and a substituted C₁-C₁₀ alkyl; and wherein A is a heteroaryl.

In some embodiments, the compound is or comprises:

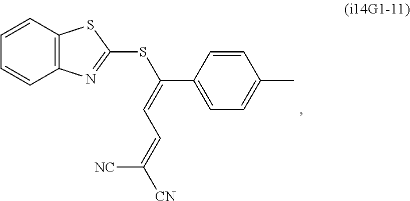 [SMILES: Cc1ccc(/C(=C\C=C(C#N)C#N)Sc2nc3ccccc3s2)cc1]

including any tautomer, any salt, or any functional derivative thereof.

In some embodiments, the compound is or comprises:

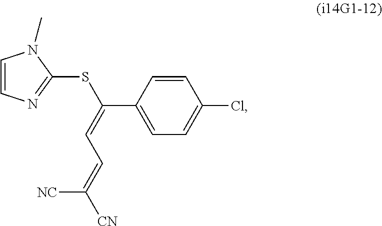 [SMILES: Cn1ccnc1S/C(=C/C=C(C#N)C#N)c1ccc(Cl)cc1]

including any tautomer, any salt, or any functional derivative thereof.

In some embodiments, the compound is selected from i14G1-10, i14G1-11, i14G12 and a salt, tautomer, functional derivative or any combination thereof. In some embodiments, the compound is i14G1-10. In some embodiments, the compound is i14G1-11. In some embodiments, the compound is i14G1-12. Compounds i14G1-10, i14G1-11, and i14G12 are all commercially available and can be purchased from Enamine. These three molecules are all found in their “REAL drug-like” library. A skilled artisan will be able to modify these three compounds to generate derivatives and similar molecules such as are recited herein using standard chemistry and organic chemistry techniques.

As used herein, the term “alkyl” describes an aliphatic hydrocarbon including straight chain and branched chain groups. In some embodiments, the alkyl group has 1 to 10 carbon atoms, 1 to 30 carbon atoms, or 5-30 carbon atoms. In some embodiments, the alkyl group is a C₁-C₆ alkyl. Whenever a numerical range e.g., “5-30”, is stated herein, it implies that the group, in this case the alkyl group, may contain 5 carbon atoms, 6 carbon atoms, 10 carbon atoms, between 5 and 20, between 5 and 25, between 5 and 30, between 10 and 20, between 10 and 25, between 10 and 30, including any range between, up to and including 30 carbon atoms. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having between 2 and 30 carbon atoms and at least one carbon-carbon double bond. The alkenyl may be substituted or unsubstituted by one or more substituents, as described hereinabove.

The term “alkynyl”, as defined herein, is an unsaturated alkyl having between 2 and 30 carbon atoms and at least one carbon-carbon triple bond. The alkynyl may be substituted or unsubstituted by one or more substituents, as described hereinabove.

As used herein the term “C₁-C₁₀ alkyl” including any C₁-C₁₀ alkyl related compounds, is referred to any linear or branched alkyl chain comprising between 1 and 10, between 1 and 2, between 2 and 3, between 3 and 4, between 4 and 5, between 5 and 6, between 2 and 10, carbon atoms, including any range therebetween. In some embodiments, C₁-C₁₀ alkyl comprises any of methyl, ethyl, propyl, butyl, pentyl, iso-pentyl, hexyl, and tert-butyl or any combination thereof. In some embodiments, C₁-C₁₀ alkyl as described herein further comprises an unsaturated bond, wherein the unsaturated bond is located at 1st, 2nd, 3rd, 4th, 5th, 6^{th}, 7^{th}, 8^{th}, 9^{th} or 10^{th} position of the C₁-C₁₀ alkyl.

In some embodiments, the compound of the invention comprises any one of the compounds disclosed herein, including any salt, any tautomer, and/or any stereoisomer (e.g., an enantiomer, and/or a diastereomer) thereof.

As used herein, the term “substituted” or the term “substituent” are related to one or more (e.g., 2, 3, 4, 5, or 6) substituents, wherein the substituent(s) is as described herein.

In some embodiments, a substituent comprises a halogen, —NO₂, —CN, —OH, —CONH₂, —CONR′₂, —CNNR′₂, —CSNR′₂, —CONH—OH, —CONH—NH₂, —NHCOR, —NHCSR, —NHCNR, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NH(C₁-C₆ alkyl), —N(C₁-C₆ alkyl)₂, C₁-C₆alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof, wherein each R′ independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof.

As used herein, the term “C₁-C₁₀ haloalkyl” refers to C₁-C₁₀ alkyl as described herein substituted by 1, 2, 3, 4, 5, 6, 7, 8, 9, 10 halide atoms, wherein halide is selected from F, Br, Cl, and I, or a combination thereof. Non-limiting examples of C₁-C₁₀ haloalkyl include but are not limited to —CF3, —CHF2, —CH2F, —CH2-CF3, —CH2-CF3, —CH2—CH2F, —CCl3, —CHBr2, —CHC12, —CBr3, —CFBrCHFBr, —CH2I, —CH2Br, —CH2Cl, —CH2—CH2I, —CH2—CH2Cl, —CH2—CH2Br, or any combination thereof.

As used herein the term “(C₃-C₁₀) cycloalkyl” is referred to an optionally substituted C₃, C4, C5, C6, C7, C8, C9 or C10 ring. In some embodiments, (C₃-C₁₀) ring comprises optionally substituted cyclopropane, cyclobutene, cyclopentane, cyclohexane, or cycloheptane.

As used herein the term “C₃-C₁₀ heterocyclyl” is referred to an optionally substituted C3, C4, C5, C6, C7, C8, C9 or C10 heterocyclic aromatic and/or aliphatic, or unsaturated ring.

In some embodiments, the term “hydroxy(C₁-C₁₀ alkyl)” and the term “C₁-C₁₀ alkoxy” are used herein interchangeably and refer to C₁-C₁₀ alkyl as described herein substituted by 1, 2, 3, 4, 5, 6, 7, 8, 9, 10 hydroxy group(s), wherein the hydroxy group(s) is located at 1^{st}, 2^{nd}, 3^{rd}, 4^{th}, 5^{th}, 6^{th}, 7^{th}, 8^{th}, 9^{th} or 10^{th} position of the C₁-C₁₀ alkyl, including any combination thereof.

In some embodiments, the compound of the invention substantially comprises a single enantiomer of any one of the compounds described herein, wherein substantially is at least 70%, at least 75%, at least 80%, at least 85%, at least 90%, at least 92%, at least 93%, at least 95%, at least 97%, at least 98%, at least 99% by weight, including any value therebetween.

In some embodiments, the compound of the invention further encompasses any structurally similar functional derivative of the compounds disclosed herein, wherein structurally similar is at least 90%, at least 95%, at least 96%, at least 97%, at least 98%, at least 99% structure similarity, including any range between.

In some embodiments, the method is a method of inhibiting binding. In some embodiments, the method is a method of inhibiting interaction. In some embodiments, the method is an in vitro method. In some embodiments, the method is an ex vivo method. In some embodiments, the method is an in vivo method. In some embodiments, the method is a method of decreasing translation initiation. In some embodiments, the eIF4G1 is in a cell. In some embodiments, the eIF1 is in a cell. In some embodiments, the decreasing is decreasing in the cell. In some embodiments, the cell is a cell of a subject.

In some embodiments, the subject is a mammal. In some embodiments, the mammal is a human. In some embodiments, the subject is a subject in need of a method of the invention. In some embodiments, the subject is suffering from a disease. In some embodiments, the disease is treatable by killing a target cell. In some embodiments, the cell is the target cell. In some embodiments, the target cell is a cell of a pathogen. In some embodiments, the target cell is a cell infected by a pathogen. In some embodiments, the pathogen is a parasite. In some embodiments, the target cell is a cell comprising increased protein expression. In some embodiments, the target cell is a cell dependent on increased protein expression for survival. In some embodiments, the target cell is a cell with increased numbers of uORFs. In some embodiments, the target cell is a cell with increased numbers of potential start codons. In some embodiments, increased is as compared to a wild-type cell. In some embodiments, a wild-type cell is a healthy control cell. In some embodiments, the wild-type cell is a non-infected cell. In some embodiments, the wild-type cell is a cell of the host. In some embodiments, the target cell is a cancerous cell. In some embodiments, the target cell is a diseased cell. In some embodiments, the diseased cell is a neuron responsible for a neurodegenerative disease. In some embodiments, the neurodegenerative disease is characterized by increased protein production.

In some embodiments, the contacting is with a compound of Formula I and the contacting increases binding of the eIF4G1 to Eukaryotic translation initiation factor 4E (eIF4E). In some embodiments, the contacting is with i14G1-10 or a derivative, salt, tautomer or functional derivative thereof and the contacting increases binding of the eIF4G1 to eIF4E. In some embodiments, the contacting is with a compound of Formula II and the contacting does not increase binding of the eIF4G1 to eIF4E. In some embodiments, the contacting is with i14G1-11 or i14G1-12 or a derivative, salt, tautomer or functional derivative thereof and the contacting does not increase binding of the eIF4G1 to eIF4E. In some embodiments, i14G1-11 or i14G1-12 is i14G1-11. In some embodiments, i14G1-11 or i14G1-12 is i14G1-12. In some embodiments, not increasing binding is not affecting binding. In some embodiments, not increasing binding is inhibits binding.

In some embodiments, the method is a method of increasing translation from an open reading frame (ORF). In some embodiments, translation is translation initiation. In some embodiments, the method is a method of decreasing translation from an ORF. In some embodiments, the ORF is an upstream ORF (uORF). In some embodiments, the ORF is an ORF with a short 5′ untranslated region (UTR). In some embodiments, the increasing translation is increasing translation from ORFs with short 5′ UTRs. In some embodiments, the ORF is an ORF with a long 5′ UTR. In some embodiments, the decreasing translation is decreasing translation from ORFs with long 5′ UTRs.

In some embodiments, a short 5′ UTR comprises at most 50, 60, 70, 75, 80, 90, 100, 110, 120, 125, 130, 140, 150, 160, 170, 175, 180, 190, 200, 210, 220, 225, 230, 240, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490 or 500 nucleotides. Each possibility represents a separate embodiment of the invention. In some embodiments, a short 5′ UTR comprises at most 150 nucleotides. In some embodiments, a long 5′ UTR is any UTR that is not a short UTR. In some embodiments, a long 5′ UTR is a 5′ UTR of greater than 50, 60, 70, 75, 80, 90, 100, 110, 120, 125, 130, 140, 150, 160, 170, 175, 180, 190, 200, 210, 220, 225, 230, 240, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490 or 500 nucleotides. Each possibility represents a separate embodiment of the invention. In some embodiments, a long 5′ UTR comprises greater than 150 nucleotides.

In some embodiments, the method is a method of increasing recognition of a non-canonical translational start codon. In some embodiments, a canonical start codon is an AUG codon. In some embodiments, the method is a method of increasing recognition of non-AUG translational start codons. In some embodiments, recognition is recognition for translation initiation. In some embodiments, recognition is recognition by the translational machinery. In some embodiments, recognition is recognition by the preinitiation complex. The preinitiation complex is well known in the art and comprises the ternary complex, the 40S ribosomal subunit and various initiation factors (e.g., eIF1, eIF1A, eIF5 and eIF3s). In some embodiments, the start codons are in the cell. In some embodiments, the start codons are in ORFs. In some embodiments, the start codons are the first translation codon. In some embodiments, first translated is first translated in a given ORF. In some embodiments, a non-canonical start codon is selected from ACG, CUG, GUG and UUG. In some embodiments, the non-AUG start codon is ACG. In some embodiments, the non-AUG start codon is CUG. In some embodiments, the non-AUG start codon is GUG. In some embodiments, the non-AUG start codon is UUG.

In some embodiments, the method is a method of increasing recognition of an AUG start codon in a weak context. In some embodiments, a weak context comprises not comprising an A or G nucleotide at position −3 from the AUG. In some embodiments, a weak context comprises having a T/U or C nucleotide at position −3 from the AUG. In some embodiments, a weak context comprises having a U nucleotide at position −3 from the AUG. In some embodiments, a weak context comprises having a C nucleotide at position −3 from the AUG. In some embodiments, the method is a method of decreasing recognition of an AUG start codon in a strong context. In some embodiments, a strong context is any context that is not a weak context. In some embodiments, a strong context comprises an A or G nucleotide at position −3 from the AUG. In some embodiments, a strong context comprises an A nucleotide at position −3 from the AUG. In some embodiments, a strong context comprises a G nucleotide at position −3 from the AUG. In some embodiments, a strong context comprises the sequence AACCAUG. In some embodiments, a strong context comprises the sequence AACCAUGGU (SEQ ID NO: 3).

In some embodiments, the method is a method of increasing recognition of the most cap-proximal AUG codon. In some embodiments, cap proximal is 3′ to the cap. In some embodiments, cap proximal is downstream of the cap. It is well known in the art that each pre-mRNA comprises a 5′ cap nucleotide. In eukaryotes the cap is a 7′methylguanylate (m7G) cap. The preinitiation complex is recruited to the 5′ cap by the eIF4F complex (eiF4E, eiF4A and eiF4G). Once the preinitiation complex is brought to the cap it begins scanning for an AUG start codon, however, the most cap-proximal AUG is not always used (in fact is frequently not used) for translation initiation. In some embodiments, the method is a method of decreasing recognition of a downstream start codon. In some embodiments, downstream is downstream of the most cap-proximal AUG codon. In some embodiments, the start codon is a canonical start codon. In some embodiments, the start codon is an AUG codon. In some embodiments, the start codon is a non-canonical start codon. In some embodiments, recognition of a more downstream start codon is leaky recognition.

In some embodiments, the method is a method of increasing translation of at least one stress-response gene. In some embodiments, a stress-response gene is a stress-response mRNA. In some embodiments, a stress-response gene is a stress-response protein. Stress response genes/proteins are well known in the art and any of the genes/proteins may have increased translation. In some embodiments, the stress-response gene/protein is an unfolded protein response (UPR) pathway gene/protein. In some embodiments, the stress-response gene/protein is an endoplasmic reticulum (ER)-stress response pathway gene/protein. In some embodiments, the stress-response gene/protein is an ultraviolet (UV)-response pathway gene/protein. Pathways and the genes/proteins that make up those pathways are well known in the art and can be found, for example, in the Gene Ontology (GO) resource, the Ingenuity Pathway Analysis software and the Kyoto Encyclopedia of Genes and Genomes (KEGG) resource.

In some embodiments, the stress-response gene/protein is Activating transcription factor 3 (ATF3). In some embodiments, the stress-response gene/protein is ATF4. In some embodiments, the stress-response gene/protein is Growth arrest and DNA damage inducible alpha (GADD45A). In some embodiments, the stress-response gene/protein is DNA damage inducible transcript 3 (DDIT3). In some embodiments, the stress-response gene/protein is Protein phosphatase 1 regulatory subunit 15A (PPP1R15A or GADD34).

Methods of detecting and quantifying gene and protein expression are well known in the art and are described hereinbelow. Any such method, including PCR, sequencing, immunoblotting and the like may be employed.

In some embodiments, the method is a method of killing the cell. In some embodiments, the cell is a target cell. In some embodiments, the cell is a cell with aberrant protein production. In some embodiments, aberrant is increased. In some embodiments, the cell is a cell with increased protein production. In some embodiments, the cell is a cell with increased numbers of uORFs. In some embodiments, the cell is a cell with increased numbers of potential start codons. In some embodiments, increased is as compared to a wild-type cell. In some embodiments, a wild-type cell is a healthy control cell. In some embodiments, the cell is a cell of a pathogen. In some embodiments, the wild-type cell is a cell of the host. In some embodiments, the host is a human. In some embodiments, the pathogen is a parasite. In some embodiments, the cell is an infected cell. In some embodiments, infected is infected with the pathogen. In some embodiments, the pathogen produces increased protein expression. In some embodiments, the pathogen is susceptible to the alteration of the start codon used. In some embodiments, alteration is the use of a more cap proximal start codon. In some embodiments, alteration is the use of a non-AUG start codon. In some embodiments, the cell is a disease cell. In some embodiments, the disease is cancer. In some embodiments, the disease is a neurodegenerative disease.

As used herein “cancer” or “pre-malignancy” are diseases associated with cell proliferation. In some embodiments, the disease is a pre-malignancy. Non-limiting types of cancer include carcinoma, sarcoma, lymphoma, leukemia, blastoma and germ cells tumors. In some embodiments, the cancer is solid cancer. In some embodiments, the cancer is a tumor. In some embodiments, the cancer is selected from hepato-biliary cancer, cervical cancer, urogenital cancer (e.g., urothelial cancer), testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer, leukemia and lymphoma. In some embodiments, the cancer is selected from breast cancer, lung cancer, colorectal cancer, ovarian cancer and bone cancer. In some embodiments, the cancer is breast cancer. In some embodiments, the cancer is lung cancer. In some embodiments, the cancer is colorectal cancer. In some embodiments, the cancer is ovarian cancer. In some embodiments, the cancer is bone cancer. In some embodiments, the cancer is a sarcoma. In some embodiments, the cancer is carcinoma. In some embodiments, the cancer is an adenocarcinoma.

In one embodiment, carcinoma refers to tumors derived from epithelial cells including but not limited to breast cancer, prostate cancer, lung cancer, pancreas cancer, and colon cancer. In one embodiment, sarcoma refers of tumors derived from mesenchymal cells including but not limited to sarcoma botryoides, chondrosarcoma, ewings sarcoma, malignant hemangioendothelioma, malignant schwannoma, osteosarcoma and soft tissue sarcomas. In one embodiment, lymphoma refers to tumors derived from hematopoietic cells that leave the bone marrow and tend to mature in the lymph nodes including but not limited to hodgkin lymphoma, non-hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refers to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but not limited to germinoma, dysgerminoma and seminoma. In one embodiment, nongerminomatous or nonseminomatous tumors refers to pure and mixed germ cells tumors including but not limited to embryonal carcinoma, endodermal sinus tumor, choriocarcinoma, tearoom, polyembryoma, gonadoblastoma and teratocarcinoma.

In some embodiments, the parasite is a malaria parasite. In some embodiments, the malaria parasite is selected from P. falciparum, P. vivax, P. ovale, and P. malariae. In some embodiments, the malaria parasite is P. falciparum. In some embodiments, the method is a method of treating malaria.

In some embodiments, the method is a method of treating a disease. In some embodiments, the disease is in a subject. In some embodiments, the disease is a pathogenic infection. In some embodiments, the pathogen is a parasite. In some embodiments, the disease treatable by inhibiting translation. In some embodiments, the disease is characterized by aberrant protein translation in a disease cell. In some embodiments, the disease is cancer. Many cancers are known to produce increased rates of protein translation as compared to healthy cells and thus are more susceptible to treatments that inhibit translation. In some embodiments, the method further comprises contacting the cell with a mammalian target of rapamycin (mTOR) inhibitor. It will be understood that while the inhibition produced by the inhibitors of the invention is distinct from mTOR inhibition, the two together have a combined effect on protein production. Thus, the inhibitors of the invention can sensitize a cell to mTOR inhibition and vice versa. mTOR inhibitors are well known in the art, and include for example, rapamycin, rapalogs, sirolimus, temsirolimus, everolimus, umirolimus, zotarolimus, torin-1, torin-2, vistusertib to name but a few. Any mTOR inhibitor may be used.

In some embodiments, the disease is a neurological disease. In some embodiments, the disease is a disease characterized by protein aggregation. In some embodiments, the disease is treatable by inhibiting translation. In some embodiments, protein aggregation is abhorrent protein aggregation. In some embodiments, the neurological disease is a neurodegenerative disease. In some embodiments, the neurodegenerative disease is Huntington's disease. The huntington gene, whose abhorrent protein expression drives Huntington's disease, is known to be regulated by a uORF and thus can be targeted by the inhibitors of the invention. In some embodiments, the disease is thrombocypenia. In some embodiments, the thrombocypenia is hereditary thrombocypenia. In some embodiments, the disease is thrombocytosis. Thrombopietin is the gene/protein responsible for both these diseases and is also known to be regulated by a uORF and can thus be targeted by the inhibitors of the invention. In some embodiments, the disease is selected from cancer, neurodegenerative disease, thrombotic diseases/disorders and parasitic infection.

In some embodiments, a functional derivative refers to a molecule capable of performing a method of the invention. In some embodiments, a functional derivative refers to a molecule capable of binding to eIF4G1. In some embodiments, a functional derivative refers to a molecule capable of blocking binding of eIF4G1 to eIF1. In some embodiments, a functional derivative refers to a molecule that inhibits binding of eIF4G1 to eIF1. In some embodiments, a functional derivative refers to a molecule capable of binding to eIF4G1 and eIF1. In some embodiments, a functional derivative refers to a molecule capable of increasing binding of eIF4G1 to Eukaryotic translation initiation factor 4E (eIF4E). In some embodiments, a functional derivative refers to a molecule incapable of increasing binding of eIF4G1 to eiF4E.

In some embodiments, inhibiting is reducing by at least 10, 15, 20, 25, 30, 35, 40, 45, 50, 55, 60, 65, 70, 75, 80, 85, 90, 95, 97, 99 or 100%. Each possibility represents a separate embodiment of the invention. In some embodiments, binding is binding at a level that is at least 10, 15, 20, 25, 30, 35, 40, 45, 50, 55, 60, 65, 70, 75, 80, 85, 90, 95, 97, 99 or 100% of the level of binding of the molecule from which it was derived. Each possibility represents a separate embodiment of the invention. In some embodiments, binding is binding at a level that is at least 50% of the level of binding of the molecule from which it was derived. In some embodiments, binding is binding at a level at least equal to that of the molecule from which it is derived. In some embodiments, inhibiting is inhibiting at a level that is at least 10, 15, 20, 25, 30, 35, 40, 45, 50, 55, 60, 65, 70, 75, 80, 85, 90, 95, 97, 99 or 100% of the level of binding of the molecule from which it was derived. Each possibility represents a separate embodiment of the invention. In some embodiments, inhibiting is inhibiting at a level at least equal to that of the molecule from which it is derived. In some embodiments, inhibiting is inhibiting at a level that is at least 50% of the level of binding of the molecule from which it was derived.

In some embodiments, a functional derivative refers to a molecule capable of inhibiting translation initiation. In some embodiments, inhibiting is decreasing. In some embodiments, a functional derivative refers to a molecule capable of increasing translation from open reading frames with short 5′ UTRs. In some embodiments, a functional derivative refers to a molecule capable of decreasing translation from open reading frames with long 5′ UTRs. In some embodiments, a functional derivative refers to a molecule capable of increasing recognition of non-AUG translational start codons. In some embodiments, a functional derivative refers to a molecule capable of increasing recognition of a most cap-proximal AUG codon. In some embodiments, a functional derivative refers to a molecule capable of decreasing leaky recognition of more downstream AUG codons than the most cap-proximal AUG codon. In some embodiments, a functional derivative refers to a molecule capable of increasing translation of at least one stress-response gene. In some embodiments, a functional derivative refers to a molecule capable of killing a contacted cell.

In some embodiments, a functional derivative refers to a molecule capable of treating a parasitic infection. In some embodiments, the parasitic infection is malaria. In some embodiments, a functional derivative refers to a molecule capable of treating malaria. In some embodiments, a functional derivative refers to a molecule capable of inhibiting the growth of a malaria parasite. In some embodiments, inhibiting growth comprises killing. In some embodiments, the malaria parasite is P. falciparum.

In some embodiments, the term “structure similarity” refers to a fingerprint similarity between two molecules. The term “fingerprint similarity” is well-understood by a skilled artisan. In some embodiments, the fingerprint similarity is calculated based on circular fingerprints, substructure keys-based fingerprints, and/or topological or path-based fingerprints.

Exemplary circular fingerprints include but are not limited to: Molprint 2D, ECFP (or Morgan fingerprint), FCFP, etc.

By another aspect, there is provided a pharmaceutical composition comprising a compound of the invention.

In some embodiments, the pharmaceutical composition comprises a pharmaceutically acceptable carrier, excipient or adjuvant. In some embodiments, the contacting is contacting with a pharmaceutical composition of the invention. In some embodiments, contacting comprises administering the pharmaceutical composition of the invention to the subject. In some embodiments, the composition comprises a therapeutically effective amount of the compound.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, or intraperitoneal.

The dosage administered will be dependent upon the age, health, and weight of the recipient, kind of concurrent treatment, if any, frequency of treatment, and the nature of the effect desired. The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the pharmaceutical composition is formulated for administration. In some embodiments, administration is administration to a subject. In some embodiments, administration is systemic administration. In some embodiments, administration is local administration. In some embodiments, the pharmaceutical composition is formulated for performance of a method of the invention. In some embodiments, the pharmaceutical composition is for use in a method of the invention.

By another aspect, there is provided a method of treating a disease, disorder or condition in a subject in need thereof, the method comprising administering to the subject a pharmaceutical composition of the invention, thereby treating the disease, disorder or condition.

By another aspect, there is provided a method of reducing translation in a cell, the method comprising reducing binding of eIF4G1 to eIF1 without increasing binding of eIF4G1 to eIF4E in the cell, thereby reducing translation in a cell.

In some embodiments, the cell is a target cell. In some embodiments, without increasing binding is with inhibiting binding. In some embodiments, the method comprises contacting the cell with a compound of the invention. In some embodiments, the method comprises contacting the cell with a compound that binds eIF4G1. In some embodiments, the binding is at an eiF1 binding site. In some embodiments, the binding occludes, blocks or makes inaccessible an eIF4E binding site. In some embodiments, the eIF4E binding site is in/on eIF4G1. Thus, it will be understood that the binding of the compound both blocks eiF1 binding but simultaneously blocks eiF4E binding thus producing a double benefit. In some embodiments, the compound is a compound represented by Formula I or a salt, tautomer, or functional derivative thereof. In some embodiments, the compound is i14G1-10 or a functional derivative thereof. In some embodiments, the compound is a compound represented by Formula II or a salt, tautomer, or functional derivative thereof. In some embodiments, the compound is i14G1-11, i14G1-12 or a functional derivative thereof. In some embodiments, the compound is i14G1-12 or a functional derivative thereof. In some embodiments, function is able to bind at an eiF1 binding site and also block/occlude a eIF4E binding site.

### General

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated.

The term “halide”, “halogen” or “halo” describes fluorine, chlorine, bromine or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” or “sulfonate” group describes an —S(═O)2-R′ group, where Rx is as defined herein.

A “carbamyl” or “carbamate” group describes an —OC(═O)—NR′R″ group, where R′ is as defined herein and R″ is as defined for R′.

A “nitro” group refers to a —NO₂ group.

The term “amide” as used herein encompasses C-amide and N-amide.

The term “C-amide” describes a —C(═O)—NR′R″ end group or a —C(═O)—NR′-linking group, as these phrases are defined hereinabove, where R′ and R″ are as defined herein.

The term “N-amide” describes a R′C(═O)—NR″— end group or a R′C(═O)—N-linking group, as these phrases are defined hereinabove, where R′ and R″ are as defined herein.

The term “carboxylic acid derivative” as used herein encompasses carboxy, amide, carbonyl, anhydride, carbonate ester, and carbamate.

A “cyano” or “nitrile” group refers to a —C≡N group.

The term “azo” or “diazo” describes an —N═NR′ end group or an —N═N—linking group, as these phrases are defined hereinabove, with R′ as defined hereinabove.

The term “guanidine” describes a —R′NC(═N)—NR″R′″ end group or a —R′NC(═N)—NR″-linking group, as these phrases are defined hereinabove, where R′, R″ and R′″ are as defined herein.

As used herein, the term “azide” refers to a —N₃ group.

The term “sulfonamide” refers to a —S(═O)2-NR′R″ group, with R′ and R″ as defined herein.

The term “phosphonyl” or “phosphonate” describes an —O—P(═O)(OR′)2 group, with R′ as defined hereinabove.

The term “phosphinyl” describes a —PR′R″ group, with R′ and R″ as defined hereinabove.

The term “alkaryl” or “alkylaryl” describes an alkyl, as defined herein, which substituted by an aryl, as described herein. An exemplary alkaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Plasmids. For bacterial expression of eIF4G1-N-RL and C-RL-eIF1 fusion proteins, the double expression plasmid pRSFDuet was used as backbone and the previously described mammalian expression plasmids encoding eIF4G1-N-RL and C-RL-eIF1 (Haimov et al., 2018, “Dynamic interactions of eIF4G1 with eIF4E and eIF1 underlie scanning dependent and independent translation”, Mol Cell Biol 10.1128/MCB.00139-18, herein incorporated by reference in its entirety) as a template for isolating eIF4G1-N-RL and C-RL-eIF1 inserts by PCR. The pAC-firefly Kozak ACG plasmid was generated using a site-directed mutagenesis kit (NEB) and pAC-firefly Kozak AUG as backbone. The pET-28-His-bdSumo-eIF4G1 (675-1129) was generated using eIF4G1 (675-1129) as insert and pET-28-14His-bdSumo as backbone in Gibson assembly reaction. The pET-28-His-eIF1 is known in the art. To generate eIF4G1 and eIF1/eIF1B sgRNA plasmids, the Benchling platform was used. Six sgRNAs were designed to target eIF4G1 exon-14 and exon-15 that encode the eIF4G1 region bearing eIF1 binding site. Similarly, 3 sgRNAs for eIF1 and 4 sgRNAs for eIF1B were targeting the last exon that is important for eIF4G1 binding. All the sgRNAs were cloned in pX459 plasmids by standard cloning protocol.

High-throughput drug screening (HTS). The HTS was done as previously described in Ashkenazi et al., 2017, “Effective cell-free drug screening protocol for protein-protein interaction”, Anal Biochem 532, 53-59, herein incorporated by reference in its entirety. The source of the compounds used are summarized in Table 1. Compounds that affected full-length RL and known RL inhibitors were dropped off from all the identified compounds. The remaining compounds were checked for their dose-response curve (0.55 μM, 1.6 μM, 5 μM, 15 μM and 45 μM) in duplicates. Further, compounds were eliminated that were common to similar HTS screening done in the drug screening facility. Final selected small molecules were then further checked in live-cell split-RL assay. The luminescence data were analyzed by GeneData software (Basel, Switzerland).

TABLE 1
Libraries used in HTS

| | | Library | Used in eIF4G1- |
|---|---|---|---|
| Supplier | Library Name | Size | eIF1 screen |
| LOPAC/SIGMA | Navigator LOPAC¹²⁸⁰ | 1280 | All |
| MicroSource | Spectrum Collection-Known Drugs | 2400 | All |
| | (66%), Natural Products (26%) and | | |
| | Other Bioactive Components (8%) | | |
| Prestwick | Prestwick Chemical Library ® - | 1200 | All |
| | approved drugs | | |
| Selleck Chemicals | Bioactive Screening Libraries | 1682 | All |
| Analyticon | MEGxp: Pure natural compounds | 3540 | All |
| | from plants | | |
| ChemBridge | DIVERSet ™ -CL | 50000 | 15 plates - 19200 |
| | | | compounds |
| Enamine | Drug-Like Set (DLS) | 20160 | All |
| MayBridge | HitFinderTM Collection | 14400 | All |

Intrinsic fluorescence of His-eIF1 and His-eIF4G1. His-eIF1 and His-eIF4G1 were purified from BL21 bacterial cells transformed with the respective plasmids. Bacteria were grown in 2YT+Kanamycin medium at 37° C. up to O.D=0.5, followed by the addition of IPTG (100 mM) and overnight incubation at 16° C. Following sonication, the soluble fraction was subjected to purification using His-select nickel affinity gel (Sigma). The purified protein was incubated i14G1-10 (0-256 uM) and i14G1-12 (0-256 uM) with shaking for 5 minutes in triplicates. After 5 minutes, fluorescence (280/350) was measured on Cytation 5 multi-mode reader (Biotek). The fluorescence intensity values corresponding to respective concentration in both i14G1-10 and i14G1-12 treatments were plotted and IC50 was calculated using GraphPad Prism 9.0.

Cells. HEK293T cells were grown and maintained in Dulbecco's modified Eagle's medium (i.e., DMEM) supplemented with 10% fetal calf serum (Invitrogen), 1% penicillin-streptomycin, 1% stable glutamine. The cells were re-plated no more than 9-10 times.

Generation of CAS9-edited NIH3T3-eIF2αAS52A cells. A 19-nt guide sequence (GCATCGTAGGCACCGTATCC (SEQ ID NO: 1)) targeting exon 3 of the mouse eIF2α gene was ligated into a pX330 hSpCas9 plasmid (#42230 Addgene, Watertown, MA). The resulting construct was cotransfected into NIH3T3 cells together with a bicistronic construct encoding from its first open reading frame a FLAG-tagged eIF2αS52A. This vector contains silent mutations that make it refractory to the guide RNA and puromycin resistance from its second open reading frame. Foci established following puromycin selection and were tested by western blot analysis using eIF2α antibodies. Foci displaying FLAG-tagged eIF2α protein instead of the endogenous protein were selected and subjected to single-cell cloning. In parallel, total genomic DNA was purified and sequenced, ensuring that the only coding DNA was that of the FLAG-eIF2αS52A vector.

Co-immunoprecipitation. HEK293T cells were transfected with 5 μg of HA-eIF1 or HA-eIF4E plasmids along with 10 ng GFP plasmid to check for transfection efficiency using Jetprime reagent as per manufacturer's protocol. After 48 h, HA-eIF1 and HA-eIF4E expressing HEK293T cells were incubated with DMSO, i14G1-10 (20 μM), and i14G1-20 (10 μM). 4 h later, cells were lysed using IP buffer (20 mM Tris pH8, 125 mM NaCl, 10% glycerol, 50 mM NaF, 1 mM Na2VO3, 0.5% NP-40, and 0.2 mM EDTA) supplemented with fresh protease inhibitor cocktail (1:100) and PMSF 200 μM (1:100). Protein extract was taken for immunoprecipitation using either monoclonal anti-HA-agarose antibody (Sigma) or control IgG antibody in IP buffer, at 4° C. for 2 hours. Each reaction was then washed 5 times with IP buffer. After the washes, each sample was eluted using 80 μl 2× sample loading buffer. 5% input and 50% of each IP sample were then subjected to 15% and 6% SDS-PAGE followed by western blot using an anti-HA antibody (for HA-tagged eIF1 and HA-tagged eIF4E) and anti-eIF4G1, respectively. For Thapsigargin and Torin treatments, the same protocol was followed wherein HA-tagged eIF1 and HA-tagged eIF4E expressing HEK293T cells were treated with either Thapsigargin (1 μM) or Torin (250 nM) for 1h.

Cap-binding Assay. HEK293T cells were grown in 15 cm culture dish until 80-90% confluence and treated with DMSO, i14G1-10 (20 μM), and i14G1-20 (10 μM). 4 h later, cells were lysed using IP buffer (20 mM Tris pH8, 125 mM NaCl, 10% glycerol, 50 mM NaF, 1 mM Na2VO3, 0.5% NP-40, and 0.2 mM EDTA) supplemented with fresh protease inhibitor cocktail (1:100) and PMSF 200 μM (1:100). Protein extract was taken binding reaction with either γ-aminophenyl-m7GTP-agarose beads (Jena Biosciences) or control empty agarose beads in IP buffer, at 4° C. for 2 hours. Each reaction was then washed 5 times with IP buffer. Elution was done using 80 μl 2× sample loading buffer. 5% input and 50% of each binding reaction were then subjected to 6% and 15% SDS-PAGE followed by western blot using anti-eIF4G1 antibody (abcam) and anti-eIF4E antibody (abeam), respectively.

Preparation of mRNA for in-vitro translation assay. Firefly luciferase gene was lifted from pAC-firefly Kozak AUG plasmid with a T7 promoter overhang containing forward primer using PCR. The firefly luciferase (1689 bp) PCR product was used as a template for in-vitro RNA transcription using RiboMAX™ large scale RNA production systems T7 (Promega) as per the manufacturer's protocol. mRNA was cleaned using the direct-zol RNA purification kit (Zymo-Research) and followed by enzymatic capping using the vaccinia capping system (NEB). Capped mRNA was cleaned using the Direct-zol RNA purification/cleaning kit (Zymo Research). The synthesized mRNA concentration was determined using Nanodrop, and the integrity was checked by agarose gel electrophoresis.

In-vitro translation. In-vitro translation reaction was set up as follow: 4 μl RRL (rabbitreticulocyte lysate (Promega, Rabbit reticulocyte lysate system L4960)), 0.5 μl amino acid mix, 50 units RNase inhibitor, (Sigma), and the indicated concentrations of i14G1-10 and i14G1-12 and incubated 10 minutes at 30° C. After 10 minutes, 40 ng Firefly luciferase capped RNA was added and incubated at 30° C. for 90 minutes. Then, Firefly luciferase luminescence was measured using Luciferase assay buffer (20 mM tricine, 0.1 mM EDTA, 1.07 mM Magnesium carbonate hydroxide pentahydrate, 2.67 mM Magnesium sulfate, 33.3 mM DTT, 270 μM Coenzyme A, 470 μM Luciferin, and 530 μM ATP) on Turner Biosystems Luminometer. The luminescence signal corresponding to each concentration of i14G1-10/12 were plotted and IC-50 was determined using Graphpad Prism 9.0.

Cell cycle and cell viability analyses. HEK293T were treated with DMSO, i14G1-10 (20 uM) and i14G1-12 (1 uM) for 16 hours and then were trypsinized, washed twice with ice cold PBS and fixed overnight in 70% ethanol. Then cells were washed twice with ice cold PBS and resuspended in staining buffer (0.1% triton X-100, 2 mg RNase A and 4% propidium iodide), and incubated at 37° C. for 15 minutes. Cells were monitored by BC LSRII flow cytometer and data was analyzed using Modfit Lt Software.

The indicated cell lines in 96-well plates were treated with increasing concentrations of i14G1-10 and i14G1-12. After 48 h, cells were subjected to cell viability measurement using CellTiter-Glo Luminescent Assay (Cat no. G7571, Promega).

### Global and Gene-Specific Translation Analysis

Polysome profile and real-time PCR. HEK293T cells were cultured in 10 cm plate up to 80% confluence, followed by DMSO, i14G1-10 (20 μM), and i14G1-12 (10 μM) treatment. After 3 h, the cells were incubated with 100 μg/ml cycloheximide for 5 minutes, washed with cold polysome buffer (20 mM Tris pH8, 140 mM KCl, 5 mM MgCl2, and 100 μg/ml cycloheximide). Cells were collected in 500-μl polysome buffer supplemented with 0.5% Triton, 0.5% DOC, 1.5 mM DTT, 150 units RNase inhibitor, and 5-μl protease inhibitor cocktail. Lysed samples were centrifuged at 12000 RPM, 5 minutes at 4° C. The cleared lysate was loaded onto sucrose density gradient (10-50%) and centrifuged at 38000 RPM for 105 minutes at 4° C. Gradients were fractionated with continuous absorbance measurement at 254 nm using ISCO absorbance detector UA-6. Fractions were pooled according to their absorbance into Free, Light, and Heavy ribosomal fractions. RNA was isolated from each respective sample using BioTri-Reagent and Direct-Zol RNA mini-prep kit (Zymo Research). cDNA was prepared from 1 μg RNA using a High-capacity cDNA reverse transcription kit (Applied Biosystems). Real-time PCR was done using qPCRBio SyGreen Blue mix (PCR Biosystems) on Quantstudio 6 Flex Real-time PCR system.

Puromycin labeling. HEK293T cells were grown in a 6-well plate until 80% confluence and incubated with DMSO, i14G1-10 (20 μM), i14G-20 (10 μM) for 2-3 hours followed by addition of puromycin (10 μg/ml) for 5 minutes. The treated cells were lysed using RIPA lysis buffer and lysates and subjected to 10% SDS-PAGE followed by western blot using anti-puromycin (Millipore).

Ribo-seq and TI-seq. HEK293T cells were grown in 10 cm culture dish until 80% confluence, and treated with DMSO, i14G1-10 (20p M), and i14G1-12 (10 μM). After 3 h, for TI-seq, cells were treated with 2 μg/ml harringtonine (Hrr) for 2 minutes followed by the addition of 100 μg/ml cycloheximide (CHX) for 5 minutes. For Ribo-seq cells were treated with 100 μg/ml cycloheximide (CHX) for 5 minutes. Thereafter, cells were lysed in polysome buffer. For both TI-seq and Ribo-seq, ribosome fractions were isolated using sucrose density centrifugation followed by Rnase I digestion. The total RNA was isolated using tri-reagent and used for high-throughput library preparation as described (Ingolia et al. 2012). The libraries were sequenced on the HiSeq2500 High-Output instrument (Illumina) for SR-60.

RNA-seq libraries. Total RNA was isolated from the lysates used for TI-seq and Ribo-seq, using Bio Tri RNA reagent. Total RNA was then cleaned up using Oligo d(T)25 Magnetic Beads (NEB 514195) to isolate mRNA. RNA-seq libraries prepared using a derivation of MARS-seq, produce expression libraries with two replicates of each treatment. ˜25 ng RNA was taken for first Reverse transcription reaction using Illumina barcoded RT1 primer. Resultant barcoded cDNA samples were subsequently pooled according to Ct values of Housekeeping gene (GAPDH) (Quality control 1). Pooled cDNA was treated with Exonuclease I to remove excess primers followed by Second strand synthesis using NEB SSS module enzyme mix. After that, in vitro transcription was performed using NEB T7 RNA Pol mix to generate RNA which was fragmented and ligated to an adaptor consisting RD2 using T4 RNA ligase I (NEB) followed by a second Reverse transcription reaction. The library was amplified using Kappa Hifi ready mix. The RNA libraries were sequenced using a high-throughput 75 bp kit (Illumina FC404-2005) on NEXTseq 500 sequencer.

### Ribo-Seq and TI-Seq Data Analysis

Preprocessing and alignment. Initial analysis steps consist of preprocessing sequences by removing the first base and adapter and filtering for sequences with cutadapt to keep sequences that had adapter (not trimming by the quality of bases). rRNA was removed by running bowtie against a database of rRNA. Next, sequences were aligned to RefSeq hg38 transcripts (downloaded from iGenomes) using bowtie (parameters -norc-S-1 25 -n 2 -m 100 -best -strata). Proportion plots of aligned reads to the length of reads was done using Ribose R-package (Bioconductor) after converting the sam file to bam with samtools. For gene quantification and RP summit detection, reads of length 28-33 were selected using cutadapt (parameters: -m 28 -M 33) and aligned to hg38 human genome using TopHat2. Bam files were converted to tdf files using igvtools count to view with IGV.

CDS and 5′UTR quantification. For quantification UTR and CDS, gtf files were created from RefSeq annotation, avoiding regions found in both region types. HTSeq was used for the quantification (parameters -s yes -t exon -m intersection-nonempty).

Translation efficiency, 5′UTR length, and start codon analysis. Counts for 5′ UTR and CDS were unified per sample, and the quartile distribution was calculated. Only genes that had at least 26 counts in one of the samples were used in subsequent steps. Variations in sample sizes were normalized using the DEseq2 statistical R language package. Translation efficiency for each sample was calculated from the ratio of ribosome profiling counts to the bulk Mars-seq mRNA levels for 5′ UTR and CDS separately. Thereupon, the ratio between samples and controls was calculated on log-transformed data. Genes were categorized into upregulated or downregulated if the sample vs control ratio of translation efficiency was over log₂ 0.8 or under log₂-0.8 for both samples, respectively, or unaffected for all other cases. The mean 5′ UTR length (for all transcripts defined in the iGenomes Illumina hg38 gtf file) per gene distribution was calculated for the aforementioned categories. Differences between gene categories were evaluated using one-way ANOVA, and the effect size was estimated using Cohen's D. Similarly, mean 5′ UTR length distribution was also performed for categories in a pairwise fashion after mutually excluding genes that belong to both categories (intersection). Start codon boundary (±6 bp) sequences were retrieved from a fasta file containing all hg38 mRNA sequences (downloaded from the NBCI nucleotide page search using “human[organism]” and filtered for mRNA and RefSeq using the left panel) using the bedtools getfasta program.

Finding Ribosome profiling summits in 5′UTR Regions enriched with RP within 5′UTR were identified using macs (parameters: -g 3e9 --keep-dup all --nomodel --shiftsize=1) with no model and no filtering of PCR duplicates. Summit peaks were annotated by the intersection (intersectBed) with RefSeq gtf file, shifted 12 bases, and expanded to 3 bases (awk commands). Bed files were derived for peaks appearing on both replicates and are within 5-UTR the region and can be extended to 9 bases within exon 5′-UTR boundaries (IntersectBed and a Perl script). The peaks bed files were divided according to gene lists (TE down UTR, TE up UTR, and TE unaffected) (dedicated Perl script), and their sequence was extracted to fasta files (bedtools getfasta). Motif enrichment search within 5′ UTR ribosome profiling extended summit sequences was done with MEME (parameters: -minw 6 -maxw 9 -RNA).

5′UTR and CDS body coverage plots. We used RSeQC geneBody_coverage.py script to plot the coverage using the Tophat bam and 5′-UTR and CDS regions bed12 format (long bed) files. A Perl script was used to create bed files for the specific gene lists.

RNA-seq (Bulk Marseq) analyses. The data analysis was performed by using the UTAP transcriptome analysis pipeline (Kohen et al., 2019). The Raw reads were trimmed using cutadapt with the parameters: -a AGATCGGAAGAGCACACGTCTGAACTCCAGTCAC (SEQ ID NO: 2) -a “A-times 2 -u 3 -u -3 -q 20 -m 25). Reads were mapped to the human genome (hg38) using STAR (v2.4.2a) with the parameters -alignEndsType EndToEnd, -outFilterMismatchNoverLmax 0.05, -twopassMode Basic, -alignSoftClipAtReferenceEnds No. The pipeline quantifies the 3′ of Gencode annotated genes (The 3′ region contains 1,000 bases upstream of the 3′ end and 100 bases downstream). UMI counting was done after marking duplicates (in-house script) using HTSeq-count in union mode. The reads with unique mapping were considered for analysis, and genes having minimum 5 reads in at least one sample were considered.

Reporter assays. 100 ng pAC-firefly Kozak NUG plasmid NUG (N=A/C/G/U) plasmid along with 100 ng Renilla plasmid (used for transfection normalization) were transfected in HEK293T cells using jetPEI reagent as per manufacturer's protocol. After 4 h, the media was replaced with media containing either DMSO or i14G1-10 (5 μM) or i14G1-12 (5 μM), and cells were incubated at 37° C. in a cell culture incubator for 16h. Next, cells were lysed using reporter assay buffer (Promega). The reporter gene assay was performed using CTZ for Renilla luciferase and luciferin reagent for firefly luciferase measurements. The luminescence was measured on Turner Biosystems Luminometer.

Parasitemia assay. The NF54 strain of the Plasmodium falciparum was used for the Survival test. Parasites were grown in RPMI medium pH 7.4, 25 mg/ml HEPES, 50 μg/ml hypoxanthine, 2 mg/ml sodium bicarbonate, 20 μg/ml gentamycin, and 0.5% (w/v) Albumax II (Invitrogen). Pooled donor red blood cells, provided by the Israeli blood bank, at 4% hematocrit, and incubated at 37° C. in a gas mixture of 1% 02, 5% CO2 in N2 were used.

From the same flask with -3-4% parasitemia, parasites were plated at 5 ml/well into six well-plates. To the trophozoite stage of development, i14G1-10, i14G1-12, or DMSO, were added and incubated at 37° C. with shaking. After one hour of incubation, from each sample, 1 ml of culture was taken. Infected RBCs (iRBC) were pelleted by centrifugation at 1500 rpm at room temperature and washed twice with warm fresh RPMI media. After the second wash iRBCs were transferred into a 12-well plate with 2 ml of warm RPMI and an additional 40 μL of uninfected RBCs (unRBC).

The next day, the parasitemia (the number of iRBCs divided by the number of total RBCs) was measured by microscopy. The gold-standard method for identifying and quantifying malaria parasites as disclosed in Murphy et al., 2013, “Malaria diagnostics in clinical trials.” Am J Trop Med Hyg., 89(5):824-39, herein incorporated by reference in its entirety, was used. Briefly, 2 μL of pelleted RBCs were spread into a monolayer to prepare a traditional blood smear, briefly immersed in methanol, then stained for 15-20 min with Giemsa (Merck). Infected RBCs were quantified by eye. No less than 30 fields of view were counted to find the final parasitemia percent.

Statistical analysis. Statistical analysis for Ribo-seq data was performed using R package as mentioned above. Wherever applicable p-values were calculated using Student's t test (typically one-tail, paired) using GraphPad Prism 9.0. The significance of the scatter plots was calculated using Pearson's correlation in a Student's t test (two tailed) using GraphPad Prism 9.0. The significance symbol in all experiments is *p<0.05; **p<0.01, ***p<0.001, ****p<0.0001.

Data and code availability. The RNA-Seq datasets generated during this study have been deposited in NCBI's Gene Expression Omnibus and are accessible through GEO Series accession number GSE166744.

Various resources used are summarized in Table 2.

TABLE 2
Resources table

| Reagent or Resource | Source | Identifier |
|---|---|---|
| Antibodies | | |
| Mouse monoclonal Anti-GFP | Abcam | Cat#ab1218 |
| Rabbit polyclonal Anti-HA | Abcam | Cat#ab9110 |
| Anti-eIF4E | Abcam | Cat#ab33766 |
| Rabbit polyclonal Anti-eIF4G1 | Abcam | Cat#ab2609 |
| Mouse monoclonal Anti-Puromycin | Merck | Cat#MABE343 |
| Anti-ATF4 | Proteintech | Cat#10835-1-AP |
| Anti-ATF3 | Abcam | Cat#ab207434 |
| Anti-GAPDH | Merck | Cat#MAB374 |
| Monoclonal anti-HA agarose beads | Sigma | Cat#A2095 |
| Normal mouse IgG | Santa Cruz | Cat#sc-2025 |
| Normal Rabbit IgG | Jackson | Cat# 111-035-003 |
| | Immunoresearch | |
| Bacterial and Virus Strains | | |
| DH-5α | NEB | Cat#C2987 |
| BL-21 | Bio-Lab | Cat#959758026610 |
| Chemicals, Peptides, and | | |
| Recombinant Proteins | | |
| γ-aminophenyl-m7GTP-agarose beads | Jena Biosciences | Cat#AC-141S |
| Protein G-conjugated Sepharose suspension | GE healthcare | Cat#17-0618-01 |
| beads | | |
| HIS-Select ® Nickel Affinity Gel | Sigma | Cat#P6611 |
| Dynabeads Oligo(dT)25 | NEB | Cat#S1419S |
| Cycloheximide | Sigma Aldrich | Cat#C7698 |
| Harringtonine | LKT Labs | Cat#H0169 |
| Puromycin | Sigma | Cat#P8833 |
| SUPERase_In | Invitrogen | Cat#AM2694 |
| RNase I | Invitrogen | Cat#AM2294 |
| Turbo DNase | Invitrogen | Cat#AM2238 |
| SYBR Gold nucleic acid gel stain | Invitrogen | Cat#S11494 |
| CircLigase ssDNA Ligase | Epicentre | Cat#CL4111K |
| Coelenterazine, native | Gold | Cat#CZ5; CAS: 55779- |
| | Biotechnology | 48-1 |
| D-Luciferin Firefly, Potassium Salt | Invitrogen | Cat# AM9515 |
| Reporter lysis buffer | Promega | Cat#E4030 |
| BioTri (RNA/DNA/protein extraction using | Bio-Lab | Cat#959758027100 |
| Trireagent) | | |
| JetPrime | Polyplus | Cat# 114-15 |
| Critical Commercial Assays | | |
| Rabbit reticulocyte lysate translation system | Promega | Cat#L4960 |
| SuperScript III Reverse Transcriptase | Invitrogen | Cat#18080-093 |
| High-Capacity cDNA Reverse Transcription | Thermo Fisher | Cat#4368814 |
| kit | Scientific | |
| Fast qPCRBIO SYBR Green Mix | PCR Biosystems | Cat#PB20.16-50 |
| KAPA Taq ReadyMix PCR Kit | Sigma Aldrich | Cat#KK1006 |
| Q5 ® Site-Directed Mutagenesis Kit | NEB | Cat#E0554 |
| Promega Wizard PCR Purification Kit | Promega | Cat# A9281 |
| RNA Clean & Concentrator kit | Zymo Research | Cat#ZR-R1017 |
| Direct-zol RNA MiniPrep kit | Zymo Research | Cat#R2052 |
| Gibson Assembly ® Master Mix | NEB | Cat#E2611S |
| Vaccinia Capping System | NEB | Cat#M2080S |
| CellTiter-Glo | Promega | Cat#G7570 |
| Experimental Models: Cell Lines | | |
| HEK293T | ATCC | N/A |
| Oligonucleotides | | |
| Primers for cloning and real-time | This Study | Please refer to Table 3 |
| Recombinant DNA | | |
| Plasmid: ΔC RL-eIF4G1 | (Haimov et al. 2018) | |
| Plasmid: ΔN RL-eIF1 | (Haimov et al. 2018) | |
| Plasmid: pET-28-His-bdSumo-eIF4G1 | This Study | |
| (675-1129) | | |
| Plasmid: pET-28-His-eIF1 | (Sinvani et al., 2015, “ Translational tolerance | |
| | of mitochondrial genes to metabolic energy | |
| | stress involves TISU and eIF1-eIF4GI | |
| | cooperation in start codon selection”, Cell | |
| | metabolism 21, 479-492, herein incorporated | |
| | by reference in its entirety.) | |
| Plasmid: pAC-firefly Kozak NUG plasmid | (Tang et al., 2017, “ Competition between | |
| | translation initiation factor eIF5 and its mimic | |
| | protein 5MP determines non-AUG initiation | |
| | rate genome-wide”, Nucleic Acids Res., Nov | |
| | 16;45(20):11941-11953, herein incorporated | |
| | by reference in its entirety.) | |
| Plasmid: ACG firefly luciferase expression | This study | |
| Plasmid: pCDNA3.1 Long 5′UTR firefly | Addgene | Cat#85490 |

| | |
|---|---|
| luciferase expression | |
| Plasmid: Pcruz-eIF1 | (Sinvani et al. 2015) |
| Plasmid: Pcruz-eIF4E | (Haimov et al. 2018) |

TABLE 3
Oligonucleotides used

| Oligo's Name | Application | Sequence (SEQ ID NO:) |
|---|---|---|
| Firefly luciferase Forward | In-vitro | aacgcaaTAATACGACTCACTATAGGGCTCT |
| | transcription | GAGCTATTCCAGAAG (4) |
| Firefly luciferase reverse | In-vitro | TTTTTTTTTTTTTTTTTTTTTTTACAATTTG |
| | transcription | GACTTTCCGCCC (5) |
| GAPDH Forward | Realtime PCR | AGGTCGGTGTGAACGGATTTG (6) |
| GAPDH Reverse | Realtime PCR | GGGGTCGTTGATGGCAACA (7) |
| ACTB Forward | Realtime PCR | CACCATTGGCAATGAGCGGTTC (8) |
| ACTB Reverse | Realtime PCR | AGGTCTTTGCGGATGTCCACGT (9) |
| SIRT1 Forward | Realtime PCR | GGAGGATAGAGCCTCACATGC (10) |
| SIRT1 Reverse | Realtime PCR | TGTTCGAGGATCTGTGCCAA (11) |
| DOLPP1 Forward | Realtime PCR | GTGCTCTATGGAGGCATCGC (12) |
| DOLPP1 Reverse | Realtime PCR | TGAGGCTTGTGTCTCGGATT (13) |
| DPF2 Forward | Realtime PCR | CTTGGACTCACAGACCGGAG (14) |
| DPF2 Reverse | Realtime PCR | GGCAGGGTAGGAGTACAGC (15) |
| MRPL35 Forward | Realtime PCR | ACCTGCAAGGAAGAAGCGAT (16) |
| MRPL35 Reverse | Realtime PCR | CATCAACGTACCAGTTTCGCC (17) |
| NDUFS6 Forward | Realtime PCR | GAGACTCGGGTGATAGCGTG (18) |
| NDUFS6 Reverse | Realtime PCR | GTGGTGCTGTCTGAACTGGA (19) |
| ATP6V1B2 Forward | Realtime PCR | CAGCCTCGCCTCACATACAA (20) |
| ATP6V1B2 Reverse | Realtime PCR | GTGCCATCCGGTAAGGTCAA (21) |
| ABCF Forward | Realtime PCR | CCCACAAACCACCTGGACAT (22) |
| ABCF Reverse | Realtime PCR | TTCGCATACCCACAACTCCC (23) |
| ATF4 forward | Realtime PCR | CCCTTCACCTTCTTACAACCTC (24) |
| ATF4 Reverse | Realtime PCR | TGCCCAGCTCTAAACTAAAGGA (25) |
| ATF3 forward | Realtime PCR | CCTCTGCGCTGGAATCAGTC (26) |
| ATF3 Reverse | Realtime PCR | TTCTTTCTCGTCGCCTCTTTTT (27) |
| DDIT3 forward | Realtime PCR | GAACGGCTCAAGCAGAAATC (28) |
| DDIT3 Reverse | Realtime PCR | TTCACCATTCGGTCAATCAGAG (29) |
| PPP1R15A Forward | Realtime PCR | AACCACGGAGGATAAAAGAACA (30) |
| PPP1R15A Reverse | Realtime PCR | CTGAACGATACTCCCAGGACC (31) |
| EIF2AK2 Forward | Realtime PCR | GCCGCTAAACTTGCATATCTTCA (32) |
| EIF2AK2 Reverse | Realtime PCR | TCACACGTAGTAGCAAAAGAACC (33) |
| HIST1H2A1 Forward | Realtime PCR | CGACAACAAGAAGACTCGCATCA (34) |
| HIST1H2A1 Reverse | Realtime PCR | TGTGCGATGGTGACTTTGCCCA (35) |
| HIST1H2BD Forward | Realtime PCR | AGCCGCAAGGAGAGCTATTCAG (36) |
| HIST1H2BD Reverse | Realtime PCR | CGCTCGAAGATGTCGTTGACGA (37) |
| HIST1H2BL Forward | Realtime PCR | GAAGGATGGCAAGAAGCGCAAG (38) |
| HIST1H2BL Reverse | Realtime PCR | CGCTCGAAGATGTCGTTGACGA (37) |
| HIST1H2BO Forward | Realtime PCR | CAAGGTGCTGAAGCAAGTCCAC (39) |
| HIST1H2BO Reverse | Realtime PCR | AGGTGATGGTCGAGCGCTTGTT (40) |
| HISTH2BK Forward | Realtime PCR | AACAAGCGCTCGACCATCA (41) |
| HISTH2BK Reverse | Realtime PCR | CCTTTGGGGTTGGGCTTTA (42) |
| ACG Luc Plasmid | Site directed | TAGGCCACCACGGCCGGATCC (43) |
| forward | mutagenesis | |
| ACG Luc Plasmid | Site directed | CTGCAGCTTAAGTTCGAGACTGT (44) |
| Reverse | mutagenesis | |
| eIF4G1 (675-1129) | PCR-Gibson | TGGTGGCCGGACAACCCTTAGC (45) |
| Fragment Forward | cloning | |
| eIF4G1 (675-1129) | PCR-Gibson | CGGATCTCATGAGAAGCGATTCAAAGTAC |
| Fragment Reverse | cloning | TAGTAGCTGG (46) |
| Pet28a-His-Sumo | PCR-Gibson | GCTTCTCATGAGATCCGGCTGCTAACAAA |
| Forward | cloning | GC (47) |
| Pet28a-His-Sumo Reverse | PCR-Gibson | GTTGTCCGGCCACCAGTCTGATGAAGCA |
| | cloning | (48) |

### TCP-Seq

HEK293T cells were grown to 90% confluence at 37° C. in a humidified incubator supplemented with 5% CO₂. Cells were treated with DMSO or 5 μM i14G1-12 for 1 hour. After treatment, cells were directly removed from the incubator and treated with 0.15% formaldehyde for 10 min on ice with slow shaking. The reaction was stopped by adding 37.5 mM glycine for 5 minutes. Media was aspirated and the plates were washed with cold PBS. Cells were lysed with fresh lysis buffer (100 mM KCl, 20 mM Tris-HCl pH 8, 1.5 mM MgCl₂, 1.5% (v/v) NP-40, 2 mM DTT) supplemented with protease inhibitor for 20 min in a cold room with slow agitation. Lysates were clarified by centrifugation at 20,000 g. RNA amount was quantified by nanodrop. 10% of the lysate was kept and snap freeze for total RNA extraction. Digestion was performed by the addition of 0.0075 U Rnase I per μg of RNA to the lysate for 30 min at room temperature. Digestion was stopped by adding a Super RNase inhibitor (ThermoFischer). Lysates were loaded onto a 5-45% sucrose gradient (100 mM KCl, 20 mM HEPES pH 7, 5 mM MgCl₂, 2 mM DTT) and centrifuged at 39000 rpm for two hours and forty-five minutes in an SW41 rotor at 4° C. Gradients were analyzed through a UV lamp (254 nm) and an Absorbance detector while being fractionated in 250 μL fractions using a Foxy Junior Fraction Collector (Isco).

### RNA Extraction Libraries Preparation

RNA from the undigested extract and the sucrose gradient fractions corresponding to the 40S were extracted by addition of 1 mL or one volume of TRIzol respectively. Samples were heated at 65° C. for 15 minutes and vortexed every five minutes. Extraction was continued by addition of chloroform and alcohol precipitation of the aqueous phase. RNA-seq libraries were prepared with the TruSeq stranded RNA Library Prep (Illumina). TCP-seq libraries were prepared as described herein by selecting footprints reads with a length ranging from 16 nt to 85 nt. Libraries were sequenced at the iGE3 Genomic Platform (UNIGE) on a Hiseq 4000.

### Mapping

For the TCP-seq libraries, adapters were trimmed with cutadapt (parameters: -a CTGTAGGCACCATCAAT -m 16) and reads were filtered by quality with TRIMMOMATIC (parameters: MINLEN:16 TRAILING:15 SLIDINGWINDOW:5:15). Reads were filtered out by successive mapping to rRNAs (UCSC) and to tRNAs (GtrRNA database) with bowtie and to lncRNAs (Ensembl) with HISAT2. Unmapped reads were finally aligned to the human MANE transcriptome (Ensemble release 108) with HISAT2 using standard parameters. Reads shorter than 23 nt with soft-clipping were discarded. Only primary alignments were kept for the analysis.

This protocol was also used to analyse the TCP-seq files from the public database. For the HeLa cells and HEK293T cells data, the adapter sequence was modified according to the library protocol used. To analyse NIH3T3 cells TCP-seq files, files for the mouse GRCm39 were generated from Ensembl release 108. S. cerevisiae data and were filtered by mapping to non-coding RNAs with bowtie and then to the genome with HISAT2 (EnsemblFungi R64-1-1.108). Finally, for S. pombe data, reads that did not map to a list of non-coding RNAs with bowtie were aligned to the transcriptome with HISAT2 (EnsemblFungi ASM294v2).

### Bioinformatic Analysis

A custom annotation file of the MANE transcripts was created from BioMart (Ensembl v108). Genes were counted with FeatureCounts. Transcripts were kept if they had a CPM ≥19 in all triplicates for either the DMSO or the i14G1-12 treated samples in the TCP-seq and if they had a CPM ≥0.75 in the RNA-seq. This left the analysis with 7062 transcripts. Most of the analysis was executed with custom C scripts.

Motif sequences were obtained with the Python package WebLogo 3.7.12. Gene ontology (GO) and reactome terms were defined by Gprofiler.

### Metagene

Metagene analysis was performed with the Deeptools package. Coverage was performed on bins of 1, normalized using CPM with exact scaling (parameters: --exactScaling --binSize 1 --normalizeUsing CPM). Plots were scaled by mRNA regions into 100 equal bins.

### Differential Initiation Analysis

Differential initiation analysis war performed with Limma. Reads from the 5′ UTR and on initiation sites were counted, normalized by CPM and used as input. A log 2 fold change of 1 and a false discovery rate of 0.05 were used as threshold.

### Leaky Scanning Score (LS)

Genes counts were obtained by aggregating reads from initiating or leaky ribosomes. Reads were defined as originating from “initiating ribosomes” if their 3′ ends were positioned between +20 to +26 nt from the start codon and their 5′ ends were located up to +1. On the other hand, “leaky ribosomes” footprints had 5′ and 3′ ends above +2 and +29, respectively. Reads per kilobase million (RPKM) were then generated by normalizing by 30 or by the CDS length the counts from initiating or leaky ribosomes. RPKM counts were used as input to calculated leaky scanning score with Voom-Limma with no normalization method. Genes were defined as non-leaky if LS<−3 or leaky if LS >3. P values were corrected with Benjamini-Hochberg method and a false discovery rate of 0.05 was used as threshold.

### Example 1: Identification of Small-Molecule Binders of eIF4G1-eIF1 Complex

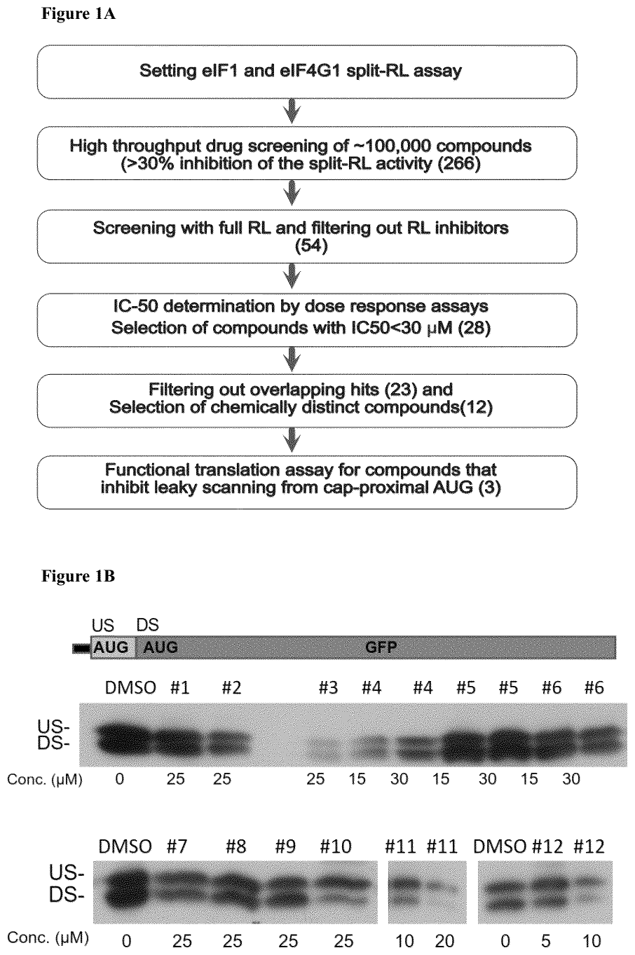

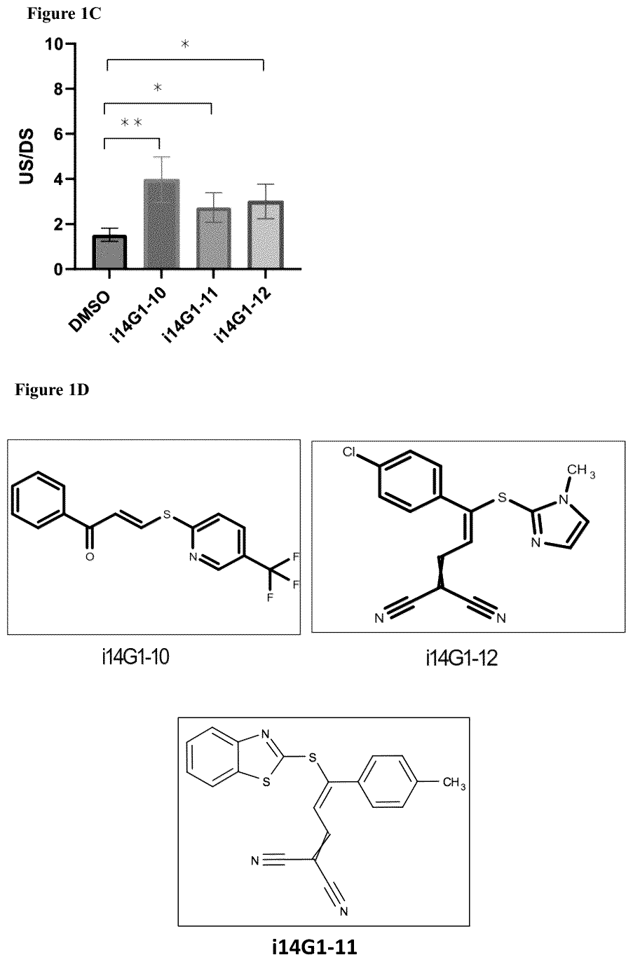

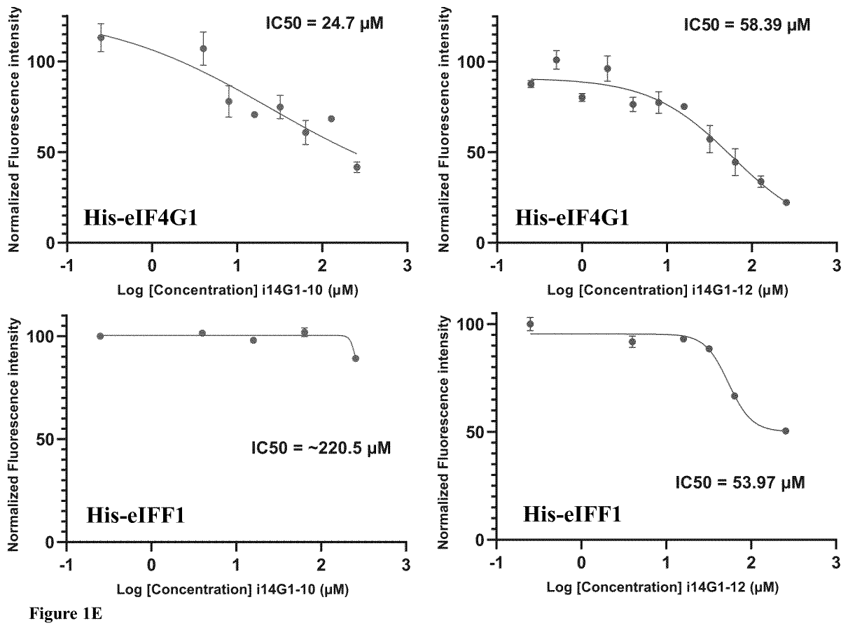

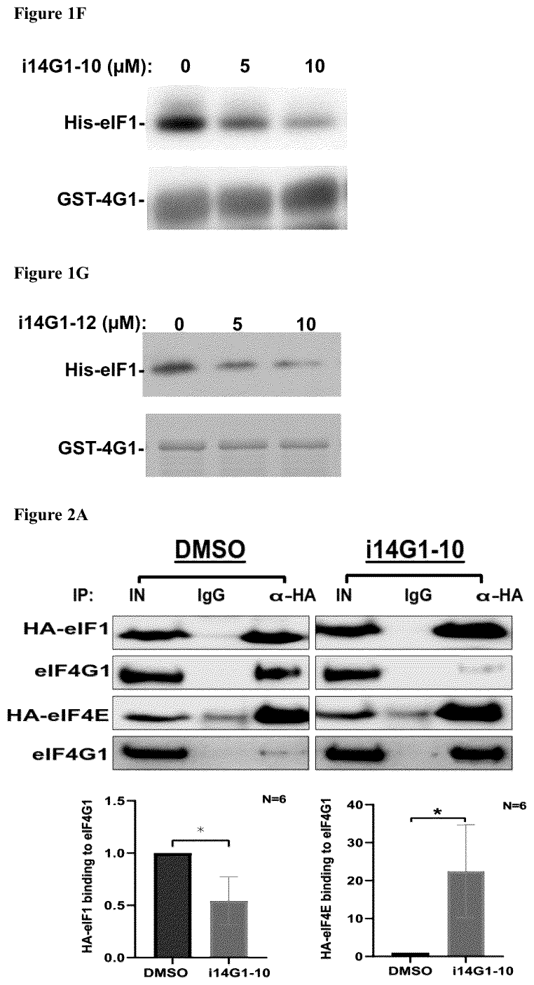

To address the functional significance of the temporal eIF1-eIF4G1 interaction, the inventors set out to develop pharmacological tools against this complex. To this end, the split-Renilla luciferase (RL) complementation assay previously found to be an efficient readout of eIF1 and eIF4G1 interaction in mammalian cells and a highly sensitive and powerful approach for the identification of protein-protein inhibitors was used. In this assay, the RL is split into two inactive N- and C-terminal fragments and fused to target proteins. Interaction of the target proteins brings the N- and C-terminal fragments of the RL in close proximity resulting in the restoration of RL activity. The eIF1-eIF4G1 split-RL fusion pair consists of the N-RL fused to the full-length eIF1 (113 amino acids) and of eIF4G1-C-RL, wherein eIF4G1 consists of amino acids 675-1129 of the protein, a region that bears the eIF1-binding site but lacks those of eIF4E, eIF4A, and eIF3. This ensures that identified binders of eIF4G1 primarily affect its interaction with eIF1. The eIF1 and eIF4G1 RL fusion proteins were expressed in bacteria from a single plasmid to enable similar expression levels. A major advantage of the use of recombinant protein is the potential identification of compounds that directly bind to a specific protein domain. Bacterial cell lysates displaying eIF1-eIF4G1 split-RL activity were used in a 1536 wells plate format to screen a ˜100,000 small molecule library (15 μM) of diverse chemical nature, for over 30% inhibition of the RL activity (FIG. 1A). A total of 266 small molecules were identified, and these were further selected for inhibition of full-length Renilla enzyme activity to eliminate RL inhibitors, resulting in 54 compounds. These small molecules were checked for overlapping hits with previous split-RL screens to filter out false positives. The remaining 28 specific eIF1-eIF4G1 split-RL inhibitors (i14G1s) were checked for IC50 and 12 compounds with an IC50<30 μM were chosen for further biological analysis (FIG. 1A). As knockdown of either eIF1 or eIF4G1 and interference with eIF1-eIF4G1 interaction led to the arrest of leaky scanning from cap-proximal AUG, the selected compounds were analyzed for their effect on initiation from cap-proximal AUG. A GFP reporter gene in which its AUG is preceded by an in-frame upstream AUG bearing a very short (16 nt) 5′UTR was used (FIG. 1B, upper panel). The translation from the downstream AUG marks leaky scanning which is ˜50% in HEK293T cells. The results show that of all the identified compounds, three molecules, #10, #11 and #12, selectively changed the upstream to downstream ratio by inhibiting leaky scanning from the cap-proximal AUG (FIG. 1B-C), reminiscent of the effect observed upon knockdown of either eIF1 or eIF4G1. Since #11 and #12 are highly similar, we selected the chemically distinct #10 and #12, for further study and named them i14G1-10 and i14G1-12 for inhibitor eIF1-eIF4G1-10 or 12. The three inhibitors are depicted in FIG. 1D.

Using a pull-down experiment, it was examined whether i14G1-10 and i14G1-12 can affect eIF1-eIF4G1 interaction. GST-eIF4G1 (aa 675-725) was incubated with His-eIF1 in the presence of vehicle (DMSO), i14G1-10 or i14G1-12. Both compounds caused a decrease in the association of eIF1 with eIF4G1 (FIG. 1F-G).

To determine to which of the proteins in the eIF1-eIF4G1 complex i14G1-10 and i14G1-12 bind, each protein was individually expressed as a His-tag fusion protein in E. coli, purified and incubated with the inhibitors followed by determination of their intrinsic fluorescence (derived from the tryptophans in eIF4G1 or the tyrosines in eIF1) as a measure of direct binding. Treatment of recombinant eIF4G1 with i14G1-10 and i14G1-12 led to a progressive loss of its intrinsic fluorescence with a calculated IC50 of 24.7 μM and 58.39 μM for i14G1-10 and i14G1-12, respectively (FIG. 1E). This indicates i14G1-12 is more potent in cells than i14G1-10. With eIF1, the intrinsic fluorescence in the presence of i14G1-10 was almost unchanged, while i14G1-12 gradually decreased it, resulting in IC-50 of 54 μM (FIG. 1E). These results suggest that within the eIF1-eIF4G1 complex i14G1-10 binds directly to eIF4G1 and i14G1-12 binds both eIF4G1 and eIF1.

### Example 2: i14G1-10 and i14G1-12 Affect the Dynamics of eIF4G1 Interaction with eIF1 and eIF4E

eIF4E and eIF1 binding sites on eIF4G1 are adjacent to each other and their interaction with eIF4G1 is mutually exclusive. To examine the effect of the novel small molecule inhibitors on the dynamics of eIF1-eIF4G1 and eIF4E-eIF4G1 complexes, co-immunoprecipitation of endogenous eIF4G1 in HEK293T cells expressing HA-eIF1 or HA-eIF4E was performed using a monoclonal anti-HA-agarose antibody. It was previously shown that exogenously expressed HA-eIF1 diminishes the expression of the endogenous eIF1 due to an auto-regulation mechanism, so the overall levels are retained. Likewise, the expression of exogenous HA-eIF4E does not lead to overexpression. It was observed that i14G1-10 (20 μM) treatment led to a 50% reduction in eIF4G1 binding to HA-eIF1 and a concomitant dramatic enhancement of eIF4G1 binding to HA-eIF4E (FIG. 2A). With i14G1-12 (10 μM, selected based on the IC-50 measurements for the two molecules) treatment, there was also observed a 25% reduction of eIF4G1 binding to HA-eIF1 but the enhancement of eIF4G1 binding to HA-eIF4E was not significant (FIG. 2B). These findings suggest that i14G1-10 acts to weaken eIF1-eIF4G1 interaction and to enhance eIF4E-eIF4G1 interactions while the effect of i14G1-12 on the eIF1-eIF4G1 complex is most likely allosteric.

As eIF4G1-eIF4E binds the m7G-cap structure of the mRNA, the effect of these drugs on their cap-binding activity was examined using immobilized 7-aminophenyl-m⁷GTP-(cap-analog) agarose beads. HEK293T cells were treated with DMSO, i14G1-10 (20 μM), or i14G1-12 (10 μM) for 4 hours and then the cells were lysed and incubated with cap-analog or control agarose beads followed by western blot to monitor eIF4E and eIF4G1. The results confirm that eIF4G1 is co-bound with eIF4E to the cap-analog beads but not to control beads. Treatment with i14G1-10 further enhanced eIF4G1 binding to eIF4E-cap complex by 1.8 folds (FIG. 2C). i14G1-12 treatment had no significant effect on the binding of eIF4G1 and eIF4E-cap complex. The co-IP and the cap-binding assays revealed that i14G1-10 shifts the eIF4G1 binding from eIF1 to eIF4E, increasing the eIF4E-eIF4G1 complex, which is crucial for ribosome recruitment. Moreover, the effect of i14G1-10 provides independent evidence for the dynamic interplay between eIF4G1-eIF4E and eIF4G1-eIF1 complexes during translation initiation.

### Example 3: i14G1-10 and i14G1-12 Inhibit Translation and Cell Growth

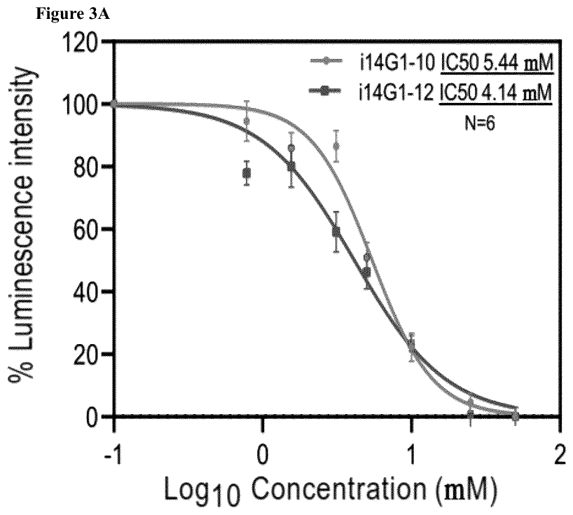

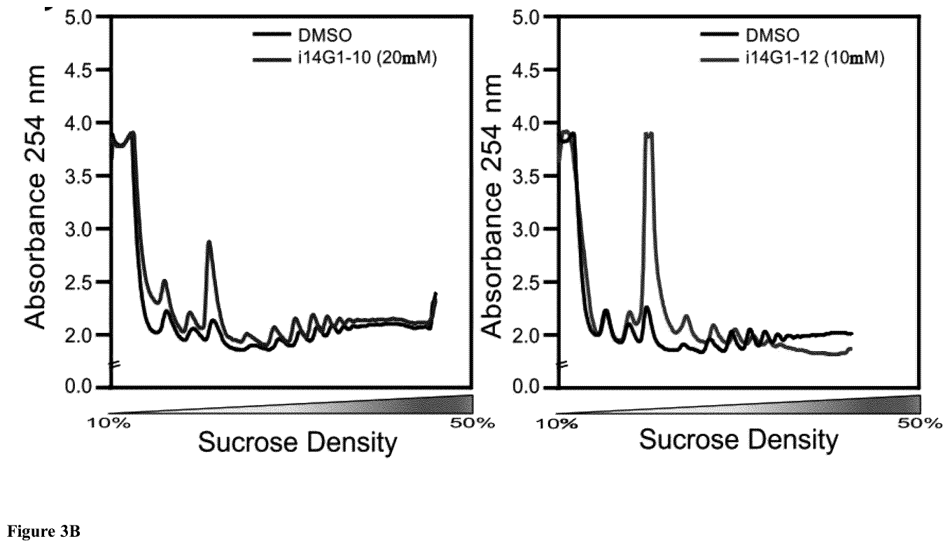

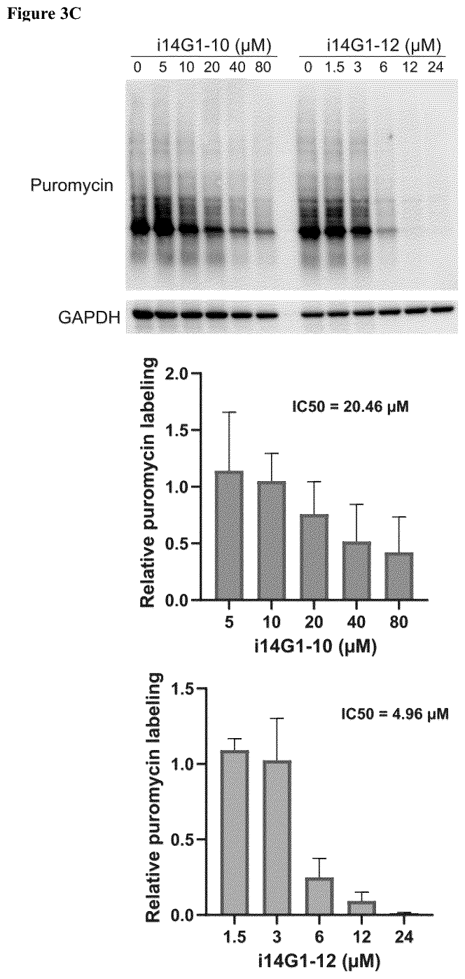

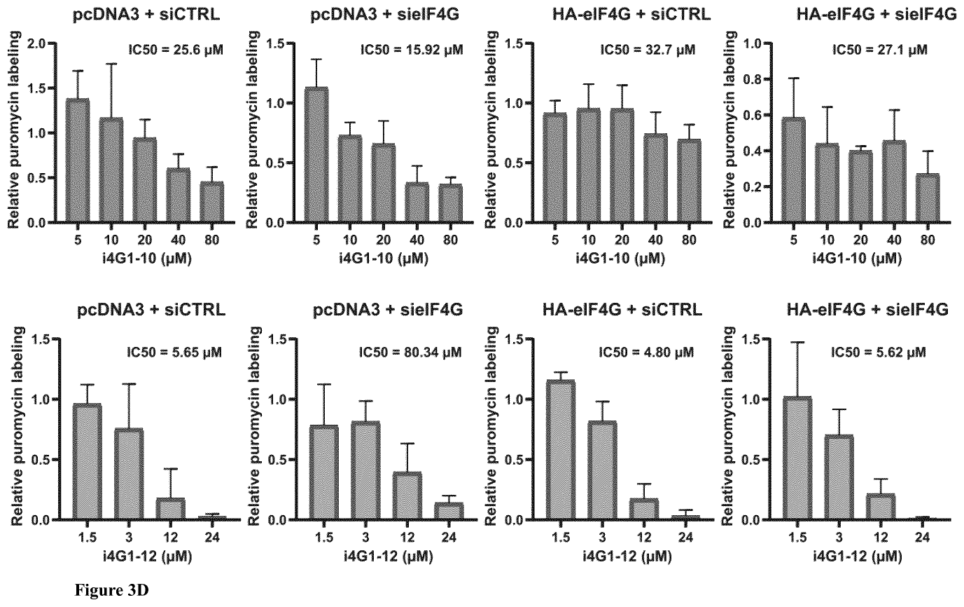

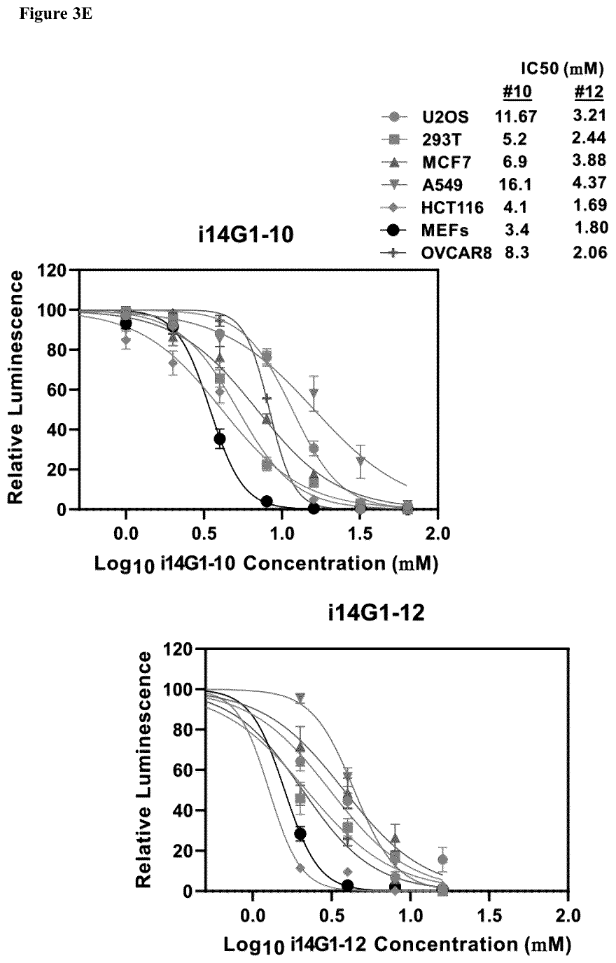

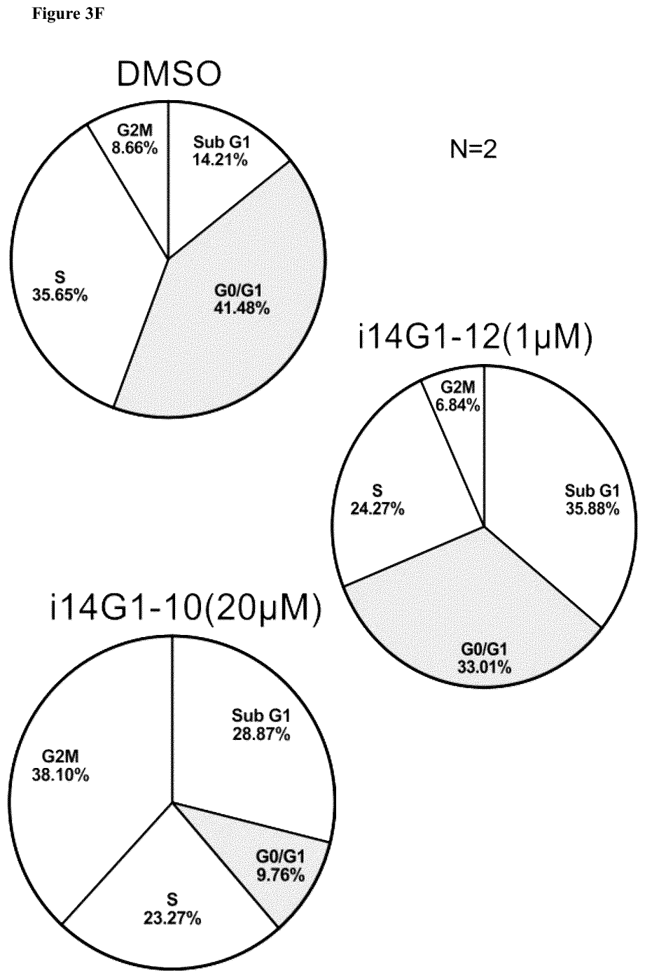

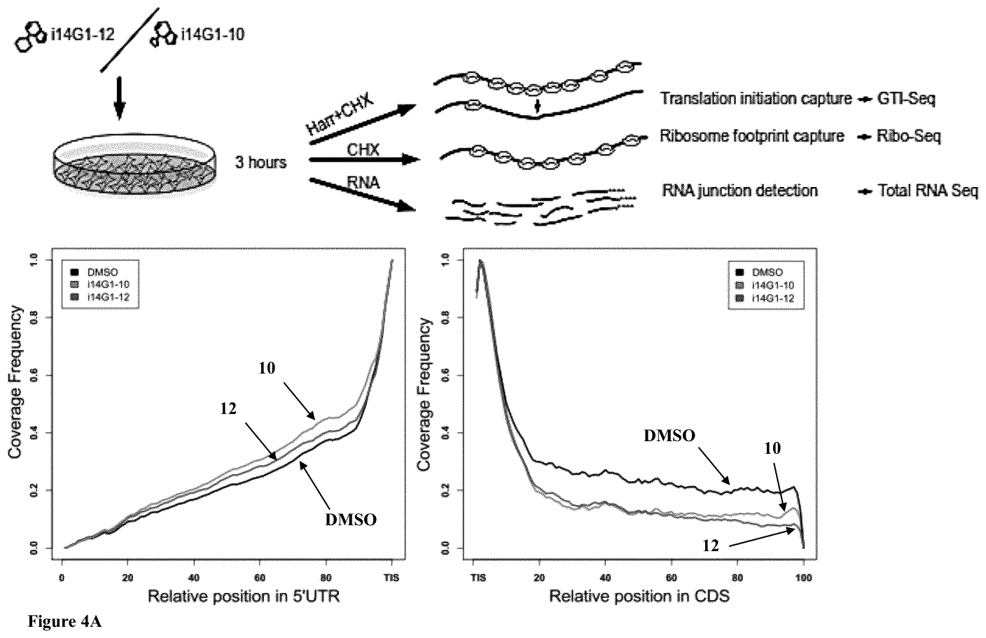

As inhibiting important interactions in the translation machinery may affect mRNA translation, the effect of i14G1-10 and i14G1-12 on in-vitro translation was examined using rabbit reticulocyte lysate and firefly luciferase mRNA. Employing increasing concentration of the two inhibitors, there was observed a dose-dependent decrease in the firefly luciferase activity, with an IC50 of 5.44 μM and 4.14 μM for i14G1-10 and i14G1-12, respectively (FIG. 3A). These findings indicate that i14G1-10 and i14G1-12 are indeed direct translation inhibitors and that eIF4G1-eIF1 interaction plays a central role during translation initiation in vitro.

To examine the effect of i14G1-10 and i14G1-12 on in-vivo translation, HEK293T cells were treated with DMSO (vehicle control), i14G1-10 (20 μM), or i14G1-12 (10 μM) for 3 h, followed by cell lysis and sucrose density gradient (10-50%) sedimentation. The polysome profiles of i14G1-10 and i14G1-12 treated samples revealed an increase in 80S monoribosomes (FIG. 3B), indicating a defect in translation initiation. With i14G1-12 there was also observed a substantial decrease in the heavy polysomal fractions suggesting that it is a more potent translation inhibitor (FIG. 3B, right). The effect of i14G1-10 and i14G1-12 on translation was also examined by applying the puromycin-incorporation assay. Puromycin is a structural analog of aminoacylated-tRNA (aa-tRNA), which leads to premature termination of translation, thus marking active translation. HEK293T cells were treated with increasing amounts of i14G1-10, or i14G1-12 for 3 h, followed by a pulse of puromycin (10 μg/ml) for 5 minutes and western blotting using an anti-puromycin antibody (FIG. 3C). The results revealed a progressive loss of nascent polypeptide labeling upon i14G1-10 and i14G1-12 treatments, with an IC-50 of 20.4 μM and 4.96 μM, respectively, further confirming a defect in de-novo protein synthesis. Here too, i14G1-12 appears a more potent translation inhibitor.

To validate that eIF4G1 is the target of these drugs in the cell, the effect of these compounds on translation was examined following eIF4G1 knockdown. Cells were transfected with control or eIF4G1 siRNA. After 48 h the cells were treated with increasing doses of i14G1-10 or i14G1-12 for 3 h, followed by 5 minutes puromycin pulse. eIF4G1 knockdown dramatically reduced the sensitivity of translation to i14G1-12, as evidenced by the change of the IC-50 from 5.65 μM to 80 μM (FIG. 3D). In contrast, with i14G1-10, the inhibition of translation was enhanced upon depletion of eIF4G1 (FIG. 3D). This enhanced sensitivity is consistent with previous studies that established that lowering the dosage of a gene whose product is targeted by a drug can result in sensitization to the drug. Transfection of eIF4G1 siRNA together with a siRNA-resistant eIF4G1 plasmid, reversed the induced sensitivity/resistance to these drugs, providing further support that eIF4G1 is a major target of these translation inhibitors.

To determine the intracellular levels of i14G1-10 and i14G1-12, cells treated with these drugs for 3 h were extensively washed and then analyzed by LC-MS. Both compounds were detected within the cells, but the amount of i14G1-12 was substantially lower than the amount that was applied (Table 4). These findings suggest i14G1-12 is either highly unstable within the cell or does not efficiently penetrate cells.

TABLE 4
Analysis of i14G1-10 and i14G1-12 uptake by LC-MS

| | Media | i14G1-10 treated cells | i14G1-12 treated cells |
|---|---|---|---|
| DMSO | — | Not detected | Not detected |
| i14G1-10 (μg/mL) | 6.2 | 1.13 ± 0.04 | Not detected |
| i14G1-12 (μg/mL) | 3.3 | Not detected | 1.22 * 10\hat{ }(−3) ± 0.015 * 10\hat{ }(−3) |

Considering the importance of mRNA translation to cell proliferation and cell survival, the effect of i14G1s on the growth of several cell lines was examined and it was found that both compounds inhibited the growth/survival of all these cell lines (FIG. 3E). This was true in breast cancer (MCF7), lung cancer (A549), colorectal cancer (HCT116), ovarian cancer (OVCAR8) and in a bone cancer sarcoma cell line (U20S). To examine whether the growth defect is associated with cell cycle progression, control and drug treated cells were subjected to DNA staining by propidium iodide and then a flow cytometry analysis to determine their distribution in the sub-G1 (dead cells), G1, S and G2/M phases of the cell cycle. The results confirm that i14G1-10 and i14G1-12 caused substantial changes in the partition of cells at the different phases of the cell cycle. Specifically, with i14G1-10 a dramatic accumulation of cells in the G2/M phase of the cell cycle was observed and with both drugs a reduction in S and elevation in sub-G1 dead cells was found (FIG. 3F).

### Example 4: The Translation Effects of i14G1-10 and i14G1-12 are Linked to Start Codon Stringencies

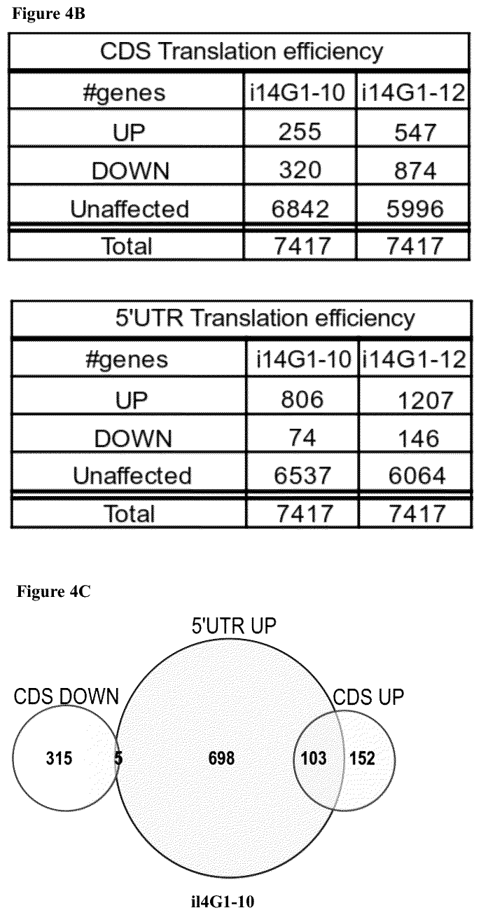

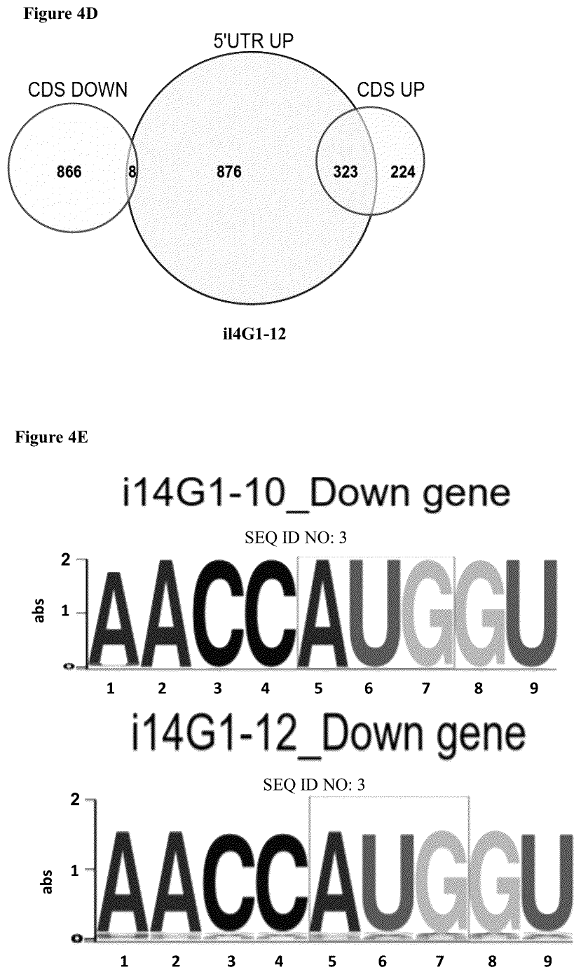

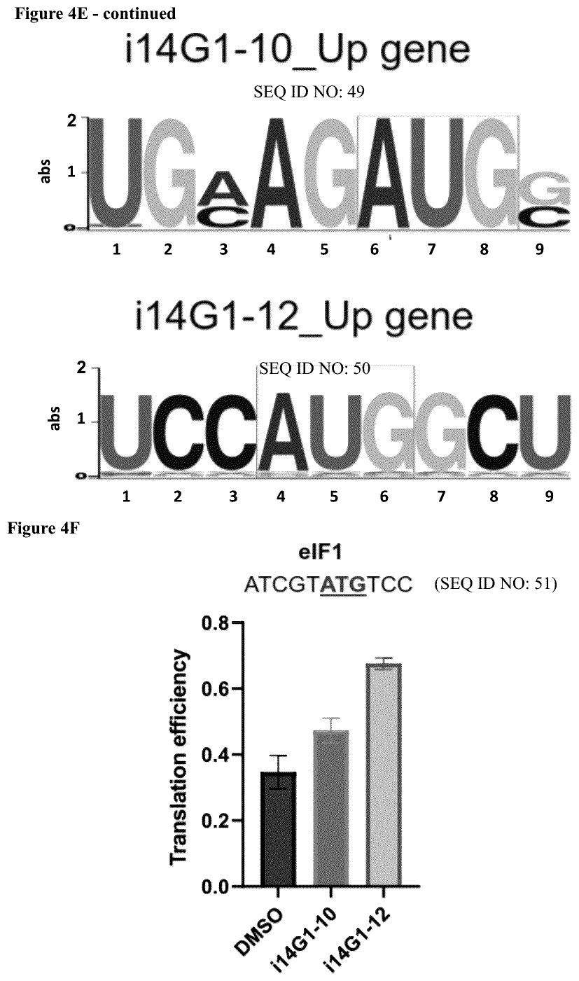

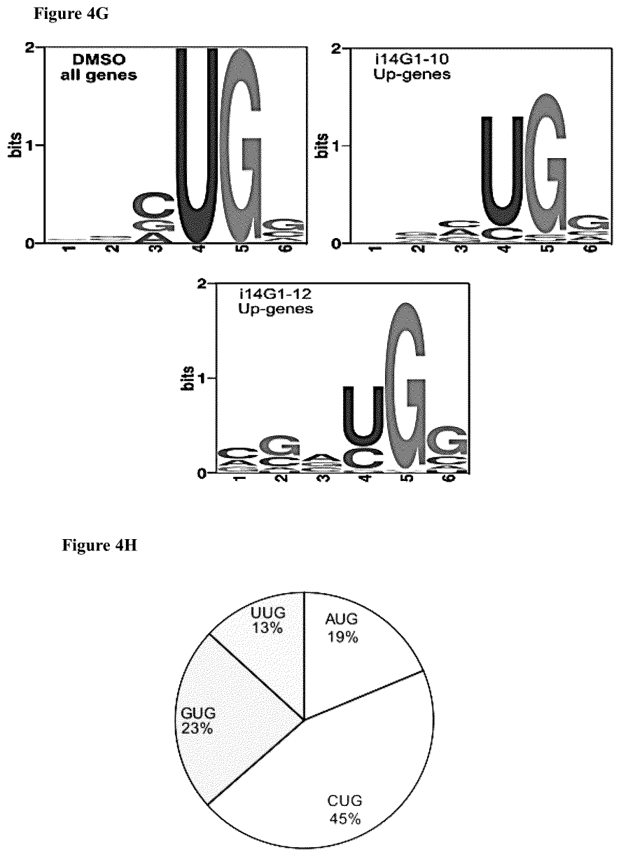

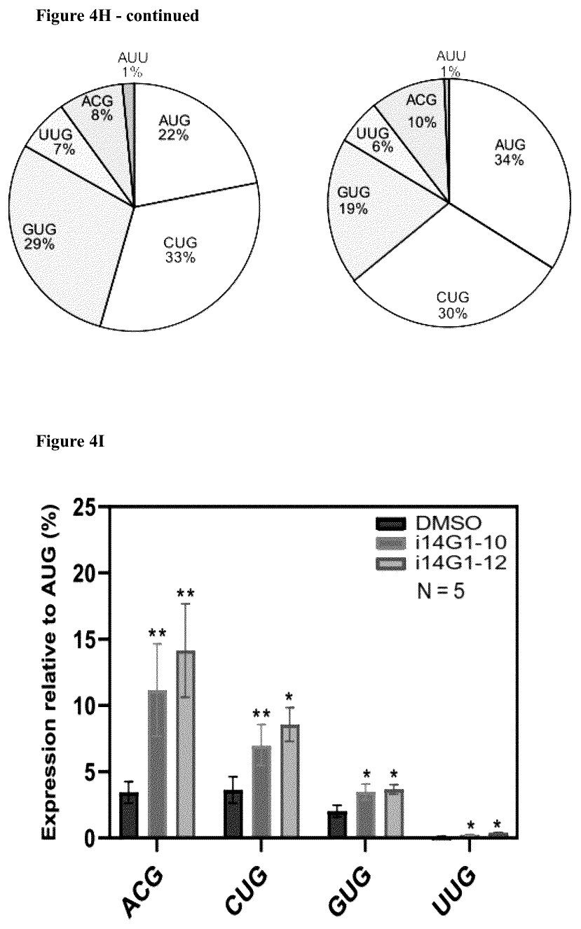

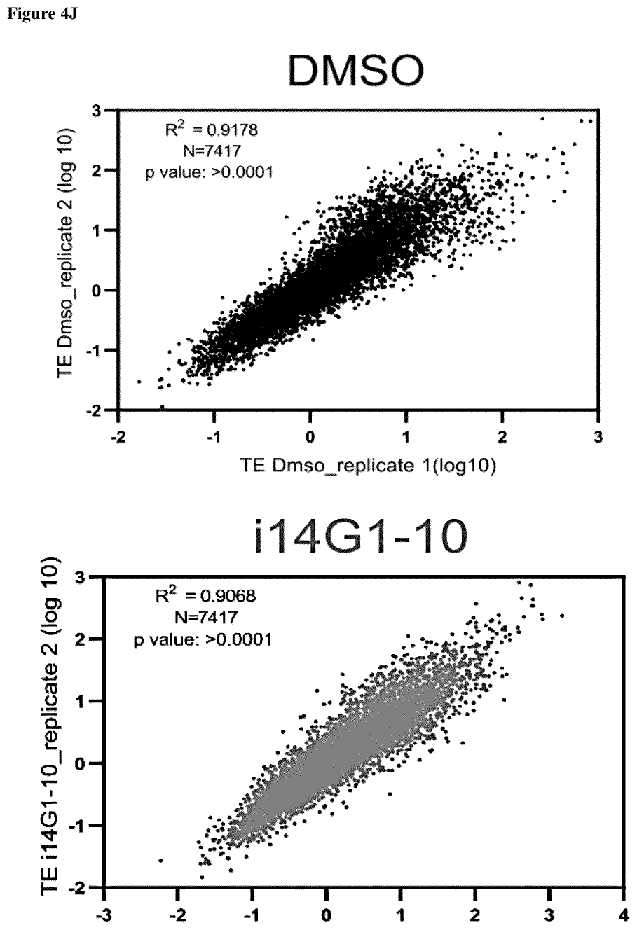

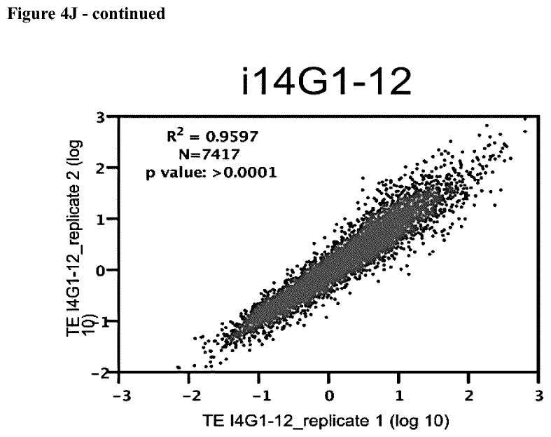

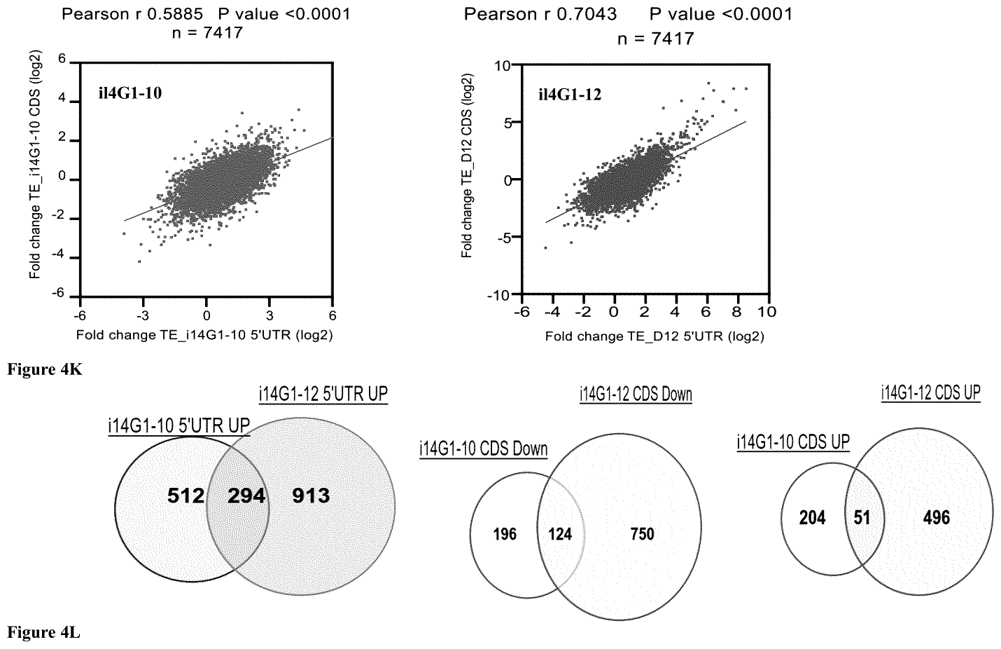

To obtain a genome-wide quantitative view of the impact of i14G1-10 and i14G1-12 on translation, Ribo-seq (deep sequencing of ribosomal protected RNA fragments) was performed following a short-term treatment (3 hours) of HEK293T cells with the compounds (FIG. 4A). For assessment of translation efficiency, cycloheximide (CHX), a translation elongation inhibitor that stalls all translating ribosomes was used. To determine the sites and regulation of translation initiation, the translation inhibitor harringtonine, which affects initiating ribosomes and enables the global mapping of translation initiation sites (GTI-seq) was also employed in addition to CHX. The analyses of total mRNA sequencing of each sample were used for normalization. There was observed a high correlation among the replicates of all the treatment groups (FIG. 4J). Metagene profiles (CHX samples) representing the mRNAs average ribosome density, revealed reduced ribosomal densities in the coding regions (CDS) for both i14G1-10 and i14G1-12 (FIG. 4A), consistent with the polysome profiles and puromycin labeling experiments described hereinabove. The i14G1-10 and i14G1-12 treatments resulted in translational downregulation of CDS (≥1.7 fold) of 320 and 874 genes, respectively, but also upregulation of 255 genes and 547 genes, respectively (FIG. 4B, top). Interestingly, elevated 5′ UTR ribosomal densities were also observed in both i14G1-10 and i14G1-12 samples (FIG. 4A), which correspond to 806 and 1207 genes with upregulated 5′UTR translation, respectively (FIG. 4B, right). As elevation in 5′UTR translation is expected to inhibit translation from the downstream major ORF, the correlation between the fold change in CDS and 5′UTR translation efficiencies was determined for 7417 expressed genes. Unexpectedly, a positive Pearson's correlation for 5′UTR and CDS translation efficiencies was found for both i14G1-10 and i14G1-12 (FIG. 4K) as was a substantial overlap between genes displaying 5′UTR upregulation and CDS upregulation, while the overlap with CDS downregulation is neglectable (FIG. 4C-D). These findings raise the possibility that in a subset of genes, inhibition of the eIF1-eIF4G1 complex enhances recruitment of the PIC to the mRNA resulting in an overall enhancement of 5′UTR and CDS translation. By comparing the extent of overlap between i14G1-10 and i14G1-12 affected genes, a considerable, albeit partial, intersection between the treatments was observed, suggesting a somewhat different mode of inhibition of the eIF1-eIF4G1 complex (FIG. 4L).

eIF1 and eIF4G1 were both shown to direct stringent AUG selection. Therefore, the sequence context of the CDS start codons in i14G1-10 and i14G1-12 translationally downregulated and upregulated genes was examined as compared to DMSO. Analysis of the nucleotide context of the annotated start codons of the differentially translated mRNAs (CDS), using the motif discovery algorithm STREME, revealed that in both i14G1-10 and i14G1-12 downregulated genes, there is a significant enrichment of a strong Kozak AUG context (AACCAUGGU (SEQ ID NO: 3)) along with additional flanking nucleotides (FIG. 4E). In contrast, the upregulated genes were significantly enriched with a weak AUG context (FIG. 4E), especially the −3 position is deviated from the A/G of the Kozak consensus. These findings suggest that the sequences surrounding the start codons are involved, at least in part, in their differential translation upon eIF4G1-eIF1 inhibition.

The translation of eIF1 itself is controlled by an evolutionarily conserved poor AUG context that is necessary for an autoregulatory negative feedback loop in which low eIF1 levels enhance its own translation and high eIF1 levels repress it. Consistent with this, it was found that the translation efficiency of endogenous eIF1 translation is upregulated upon inhibition of eIF4G1-eIF1 by both i14G1-10 and i14G1-12 (FIG. 4F).

Next, the sequence context of the translation initiation site (TIS) in the ribosome footprints of the 5′UTR in DMSO, i14G1-10 or i14G1-12 samples was determined. To assign the 5′UTR TISs, the Hrr+CHX reads were used (see Materials and Methods). The results revealed that the 5′UTR TIS consists of mostly NUG, in which the first position is the most variable. This finding is in line with other reports demonstrating that translation initiation in the 5′UTR is more flexible and can utilize both AUG and near cognate AUG. Upon both i14G1-10 and i14G1-12 treatment, the translation initiation sites become substantially more flexible with variations also in the second and third positions (FIG. 4G). Analysis of the percentage of all possible combinations of AUG single substitutions further highlights the reduced stringency and the appearance of ACG and AUU near-cognate codons in the drug-treated samples which were barely identified in DMSO control (FIG. 4H). To validate this observation further, a reporter assay was performed using a set of luciferase reporter genes in which the initiating triplet was either AUG, CUG, GUG, UUG, or ACG. HEK293T cells were transfected with the respective luciferase reporters and treated with i14G1-10 (5 μM) or i14G1-12 (5 μM) for 16 h, followed by luminescence measurements. Both i14G1-10 and i14G1-12 drug treatment significantly enhanced the activities of ACG, CUG, GUG, and UUG relative to AUG. Notably, with both drugs, the most affected initiation codon is ACG, consistent with the marked elevation of this codon in the ribosome-profiling data (FIG. 4I). Taken together, these results suggest that eIF1-eIF4G1 interaction is required to inhibit initiation from weak AUG contexts or from near-cognate start codons during 5′UTR scanning in mammalian cells. This conclusion is in full agreement with studies from yeast in which mutations in either eIF1 or eIF4G1 enhanced initiation from near-cognate AUGs.

### Example 5: 5′UTR-Length-Dependent Effects of i14G1-10 and i14G1-12

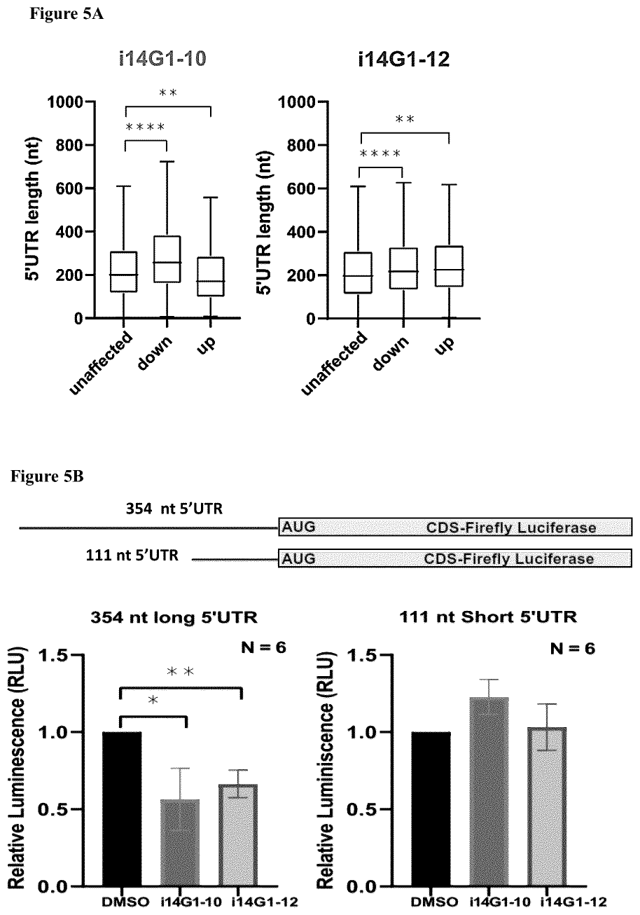

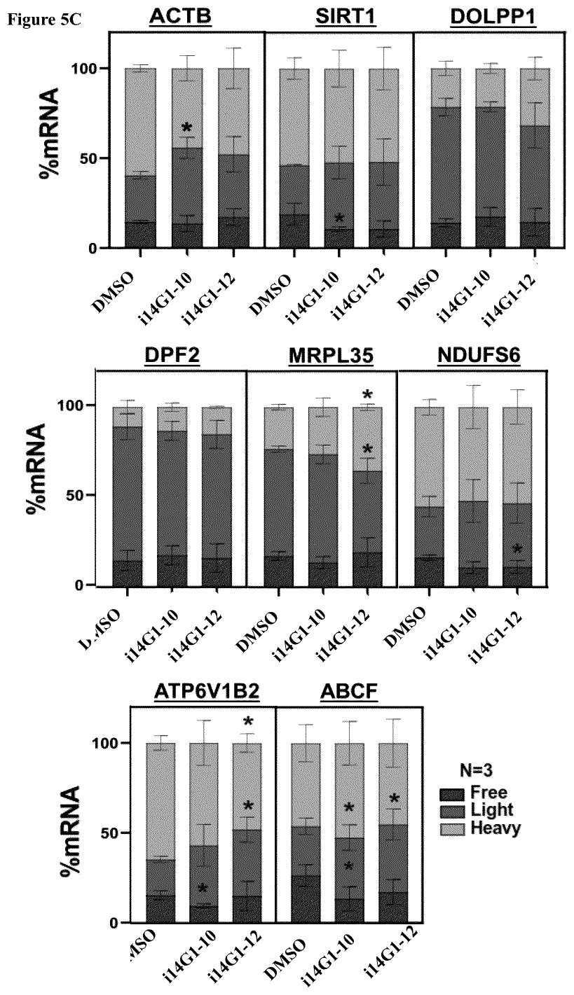

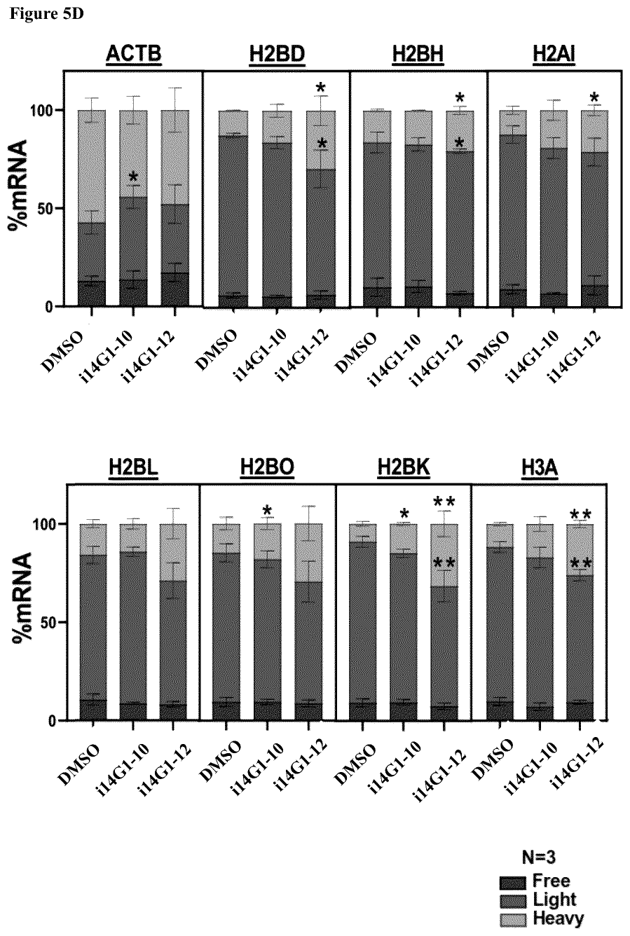

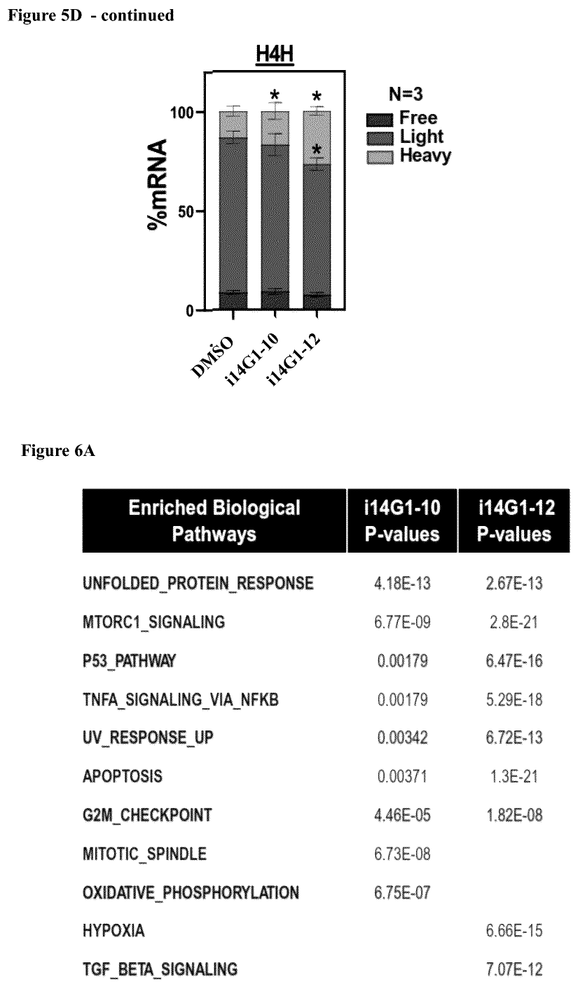

Since both eIF1 and eIF4G1 are important for scanning, the relationship between the translational effects of the inhibitors and the 5′UTR length was examined. It was observed that in both i14G1-10 and i14G1-12 treated samples, translationally downregulated mRNAs have significantly longer 5′UTR than DMSO (FIG. 5A). To investigate further the link between the effect of the drug and 5′UTR length, we used two firefly luciferase reporters we used that differ only in their 5′UTR length: a short 5′UTR with 111 nucleotides and a long 5′UTR with 354 nucleotides. While the luciferase reporter expression driven by the longer 5′UTR was significantly decreased upon both i14G1-10 and i14G1-12 treatments (FIG. 5B, left), these compounds did not affect the shorter 5′UTR (FIG. 5B, right). These findings confirm the inhibitory effect of these compounds on the scanning promoting activity of the eIF1-eIF4G1 complex.

As previous studies implicated an inhibitory role of eIF1 and eIF4G1 on scanning independent cap-proximal initiation, the inventors wished to determine whether these inhibitors recapitulate these effects on endogenous mRNAs. However, the Ribo-seq protocol involves the isolation of 30-32 nt ribosome protected fragments, which are largely excluded when the start codons are preceded by an extremely short 5′ leader. Thus, this approach is limited in its ability to report the translation state of mRNAs bearing very short 5′UTR such as TISU and histone genes. Therefore, polysomal profiling was performed following i14G1-10 or i14G1-12 treatment and seven randomly selected TISU genes in the free, light, and heavy polysomal fractions were checked by real-time PCR (FIG. 5C). All the analyzed TISU genes were either unaffected or slightly upregulated in i14G1-10 and i14G1-12 treated polysomes fractions. In contrast, both i14G1-10 and i14G1-12 decreased translation of the ACTB mRNA that was used as a control. Similarly, the translation of eight histone genes that are driven by extremely short 5′UTR and a weak AUG context were checked. Indeed, the analyzed histone genes display a general trend of upregulation following both i14G1-10 and i14G1-12 treatments (FIG. 5D). Enhanced translation of histones genes is consistent with enhanced cap-proximal translation initiation possibly due to enhanced eIF4G1-eIF4E complex relative to eIF4G1-eIF1.

### Example 6: eIF4G1-eIF1 Inhibitors Reveal its Central Role in Stress Responses

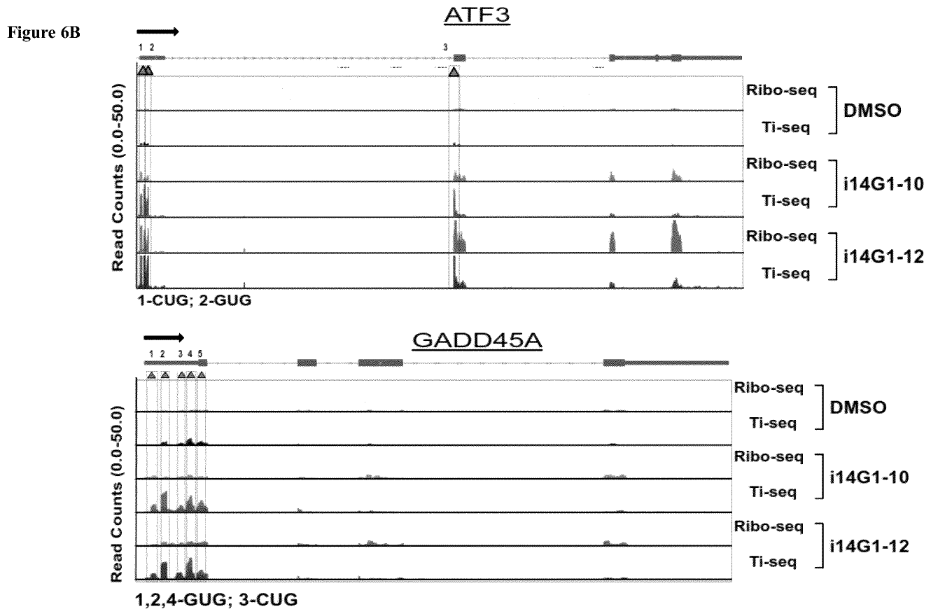

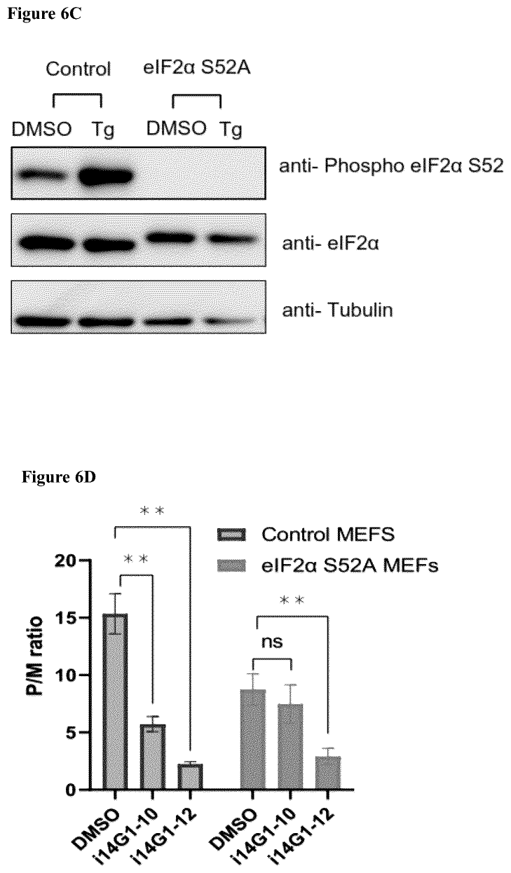

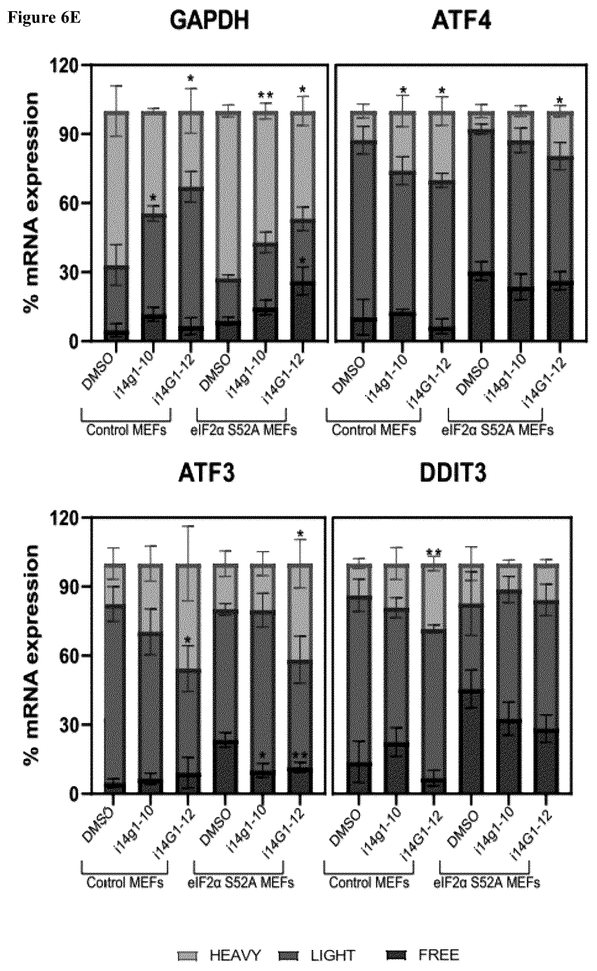

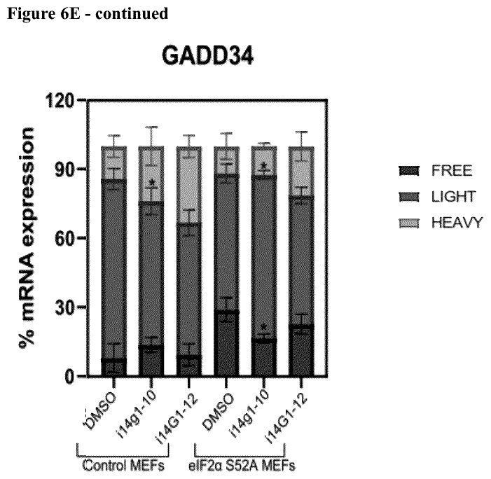

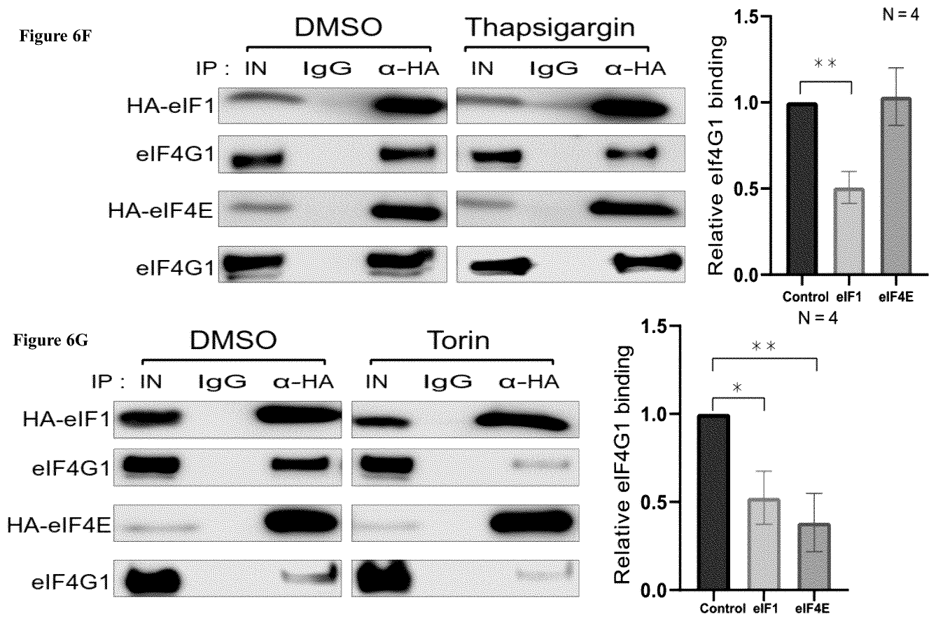

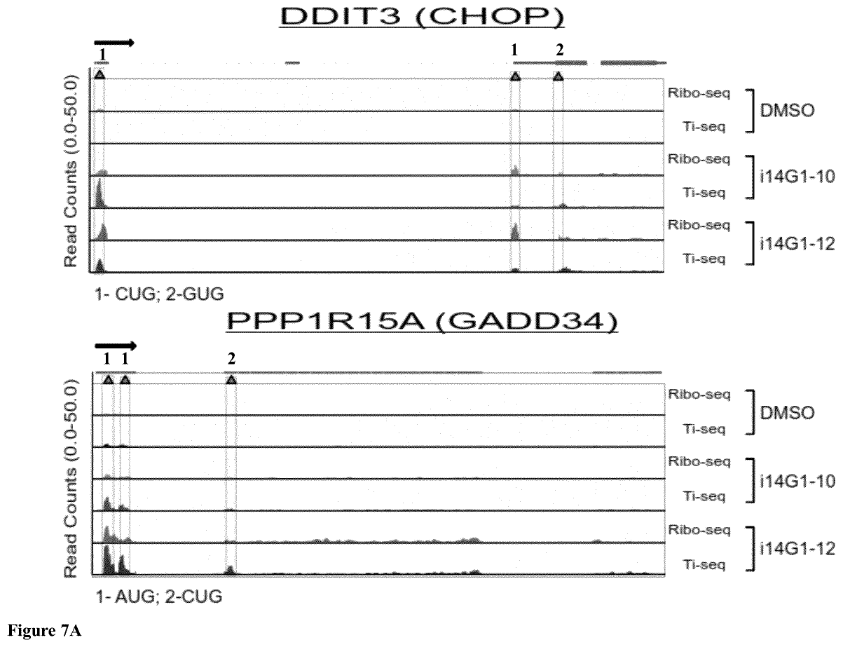

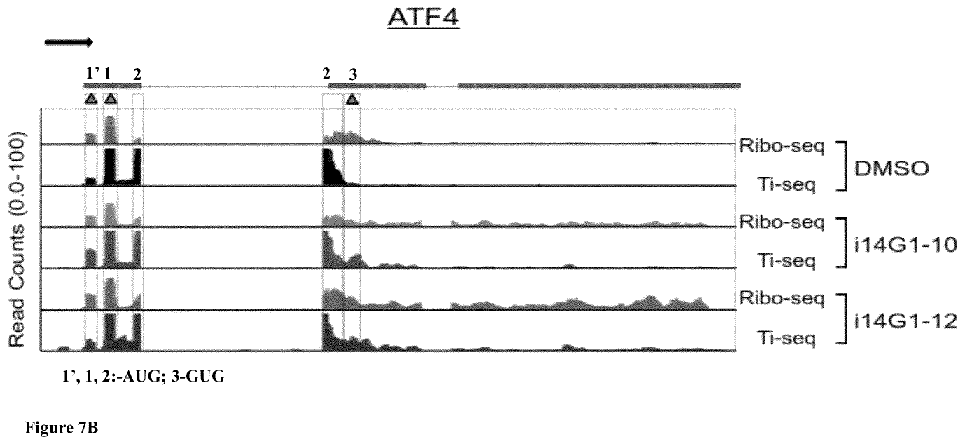

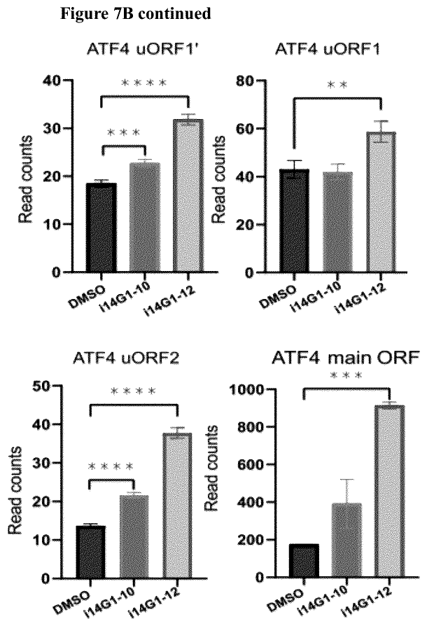

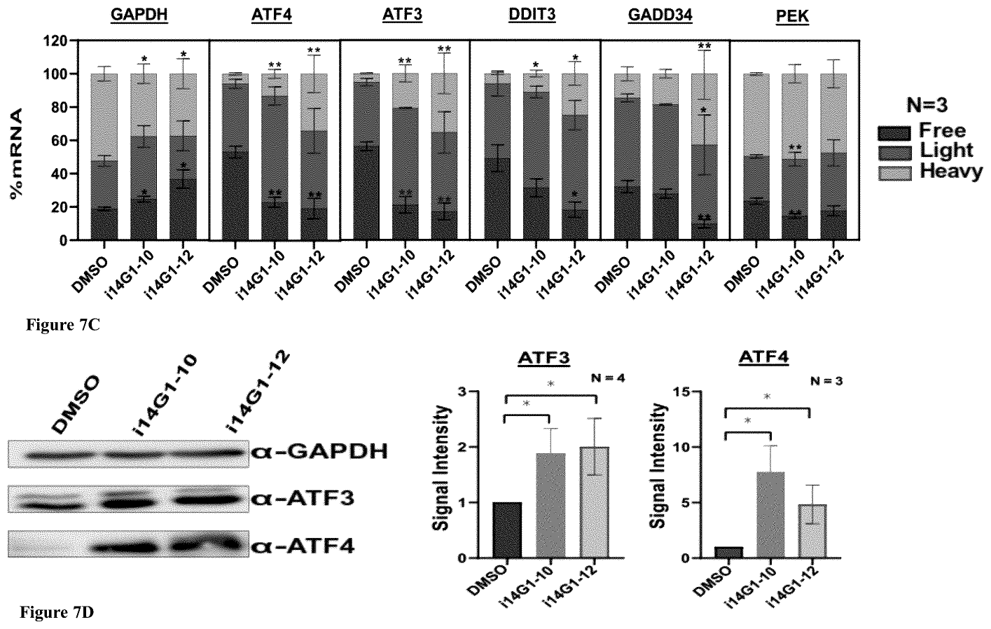

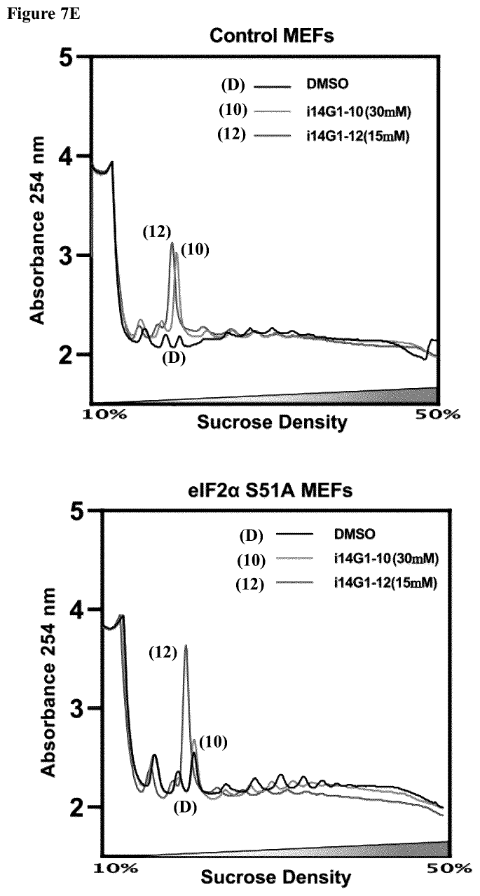

The i14G1-10 and i14G1-12 differentially translated genes were analyzed using the Ingenuity Pathway Analysis (IPA) in order to identify the major biological themes and their relationships among the affected genes. The resulting networks revealed activated genes and pathways involved in cell death and unfolded protein response (UPR) in both drug treatments while those of the downregulated genes are largely different. Likewise, using the Gene Set Enrichment Analysis (GSEA), there was found substantial overlap of affected pathways among the upregulated gene sets of both drugs that includes UPR, UV-response, apoptosis, mTORC signaling, TNFα signaling and G2/M checkpoint (FIG. 6A). The gene track of several upregulated UPR genes ATF3, GADD45A, DDIT3 and GADD34 was analyzed and it was noticed that in parallel to the increase of the reads in the CDS, there is a substantial upregulation of ribosome footprints in their 5′UTR (FIGS. 6B and 7A). As these drugs elevate eIF4E-eIF4G1 levels and the cap-binding activity (FIG. 2A-C), it is likely that the large increase of 5′UTR footprints in these mRNAs is a consequence of enhanced ribosome recruitment to the cap, of which a fraction eventually reaches the CDS via leaky scanning and re-initiation.

ATF4 is a major regulator of stringent translation control during the integrated stress response. Its translational control is distinctive from the above-described stress response gene by exhibiting a high level of 5′UTR translation under basal condition. Specifically, it has two highly conserved uORFs in its 5′UTR. The first uORF is short and is present near the 5′ end (uORF1), and the second (uORF2) starts just upstream of the main start site and overlaps the main CDS ORF. ATF4 uORF1 allows reinitiation at uORF2 provided that the recycling ribosome stays on the mRNA along with other initiation factors. Initiation from uORF2 suppresses the expression of main ORF to maintain low basal levels of ATF4. The induction of ATF4 occurs upon stress, when eIF2α is phosphorylated and the ternary complex (TC) becomes limited, leading to a reduction in uORF2 reinitiation and a delayed reinitiation at the main ORF. Since it was found that ATF4 main ORF translation is upregulated by both drugs (FIG. 7B), we closely analyzed the ribosomal footprints landscape at the individual translation start sites of ATF4. Our findings revealed the presence of an uORF that precedes uORF1, located 20 nt from the 5′end that we named uORF1′. Treatment of both i14G1-10 and i14G1-12 caused elevation in the cap-proximal uROF1′ footprints as well as uORF1 and uORF2 (FIG. 7B). Under ER/UPR stress the enhancement of translation from the main ORF is expected to be accompanied by reduced uORF2. However here, uORF2 is actually upregulated alongside the main ORF (FIG. 7A). A possible interpretation of this finding is that the enhanced overall 43S loading upon eIF1-eIF4G1 inhibition, causes a fraction of the ribosomes to bypass uORF2 and initiate at the main ORF, in a similar fashion of the other stress response genes described in FIG. 6B and FIG. 7A. Another possibility considers studies in yeast demonstrating reduced TC availability upon eIF1 inactivation, raising the possibility eIF1-eIF4G1 inhibition by the drugs also reduces TC levels and this facilitates delayed re-initiation at the main ORF. These possibilities are not mutually exclusive and maybe even additive. The translation upregulation of the various stress-response genes was validated by analyzing their distribution in the Free, Light, and Heavy fractions of polysome profiles of i14G1-10 and i14G1-12 and they were all found to be upregulated (FIG. 7C-D). Thus eIF1-eIF4G1 inhibition of 5′UTR translation is specific to a subset of stress-response genes.

### Example 7: Activation of ISR Genes by eIF4G1-eIF1 Inhibitors is eIF2α Phosphorylation Independent

To study further the activation of the integrated stress-response following eIF1-eIF4G1 inhibition, the inventors set out to examine whether the effect of eIF4G1-eIF1 inhibitors is linked to eIF2α phosphorylation. A mouse NIH3T3 cell line in which the endogenous eIF2α was deleted and replaced with a flagged-tag non-phosphorylated version of eIF2α, of which serine 52 is substituted with alanine (S52A) was used. This mutant eIF2α is refractory to thapsigargin induced-phosphorylation, a known inducer of the integrated stress response (FIG. 6C). The parental and eIF2α S52A mutant cells were subjected to polysome profiling in the absence or presence of i14G1-10 and i14G1-12 and overall translation was determined by calculating the polysome to monsome ration (P/M). As expected, there was observed a significant reduction in translation efficiency with i14G1-10 and i14G1-12 in the parental cells (FIGS. 6D and 7E). With the S52A mutant cells, it was found that the basal translation level is lower than that of the parental cells, suggesting that the S52 of eIF2α is required for optimal translation. The translation in these cells is further inhibited by i14G1-12 but not significantly with i14G1-10 (FIGS. 6D and 7E). The levels of several selected ISR genes were then determined in the free, light, and heavy polysomal fractions by real-time PCR (FIG. 6E). While the translation of the control GAPDH gene was reduced by the drugs in both cell lines, all the analyzed ISR genes were upregulated in i14G1-10 and i14G1-12 treated polysomes fractions in both the parental and the S52A cells, as evident from the shifts between heavy, light and free fractions (FIG. 6E). However, the extent of activation is lower in the mutant cells as compared to the parental cells, perhaps due to the limited basal translation potency of these cells. These findings suggest that the effect of i14G1-10 and i14G1-12 on ISR genes is, at least in part, independent of eIF2α S52 phosphorylation.

### Example 8: eIF4G1-eIF1 Interaction is Inhibited Upon Stress

The translation activation of stress response upon disturbance of eIF4G1-eIF1 interaction raises the possibility that interference with eIF4G1-eIF1 may be a previously unknown translation regulatory mechanism of stress response along with the well-known eIF2α phosphorylation. To test this possibility, a short-term (1 hour) ER stress was applied using Thapsigargin and then the eIF4G1-eIF1 interaction was analysed using co-immunoprecipitation. Remarkably, the level of co-precipitated eIF4G1 with HA-eIF1 is reduced by 50% upon ER stress (FIG. 6F). Under these conditions, no significant change in the level of co-precipitated eIF4G1 with HA-eIF4E was observed. Thus, translation upregulation of a subset of ER/UPR stress response genes may also be the consequence of diminished eIF4G1-eIF1 interactions. This prompted the examination of the fate of the eIF4G1-eIF1 complex following inhibition of mTOR, a kinase controlling eIF4E interaction with eIF4G1 via eIF4BP1 phosphorylation. Remarkably, it was found that one-hour mTOR inhibition diminishes eIF1 interaction with eIF4G1 (FIG. 6G). Collectively, these findings uncover eIF4G1-eIF1 complex formation as a regulatory target for the major stress-response pathways.

### Example 9: Treating Malaria with eIF4G1-eIF1 Inhibitors

Malaria is a deadly parasitic infection spread by mosquitos. Four kinds of malaria parasites are known to infect humans, with the most virulent being Plasmodium falciparum. In P. falciparum ˜98% of the mRNAs have an average of ˜10 uORFs per coding sequence. This unusual large number of uORFs has serious implications for translation of downstream sequences, AUG selection and overall translation. The sensitivity of P. falciparum to the inhibitors of the invention was therefore tested.

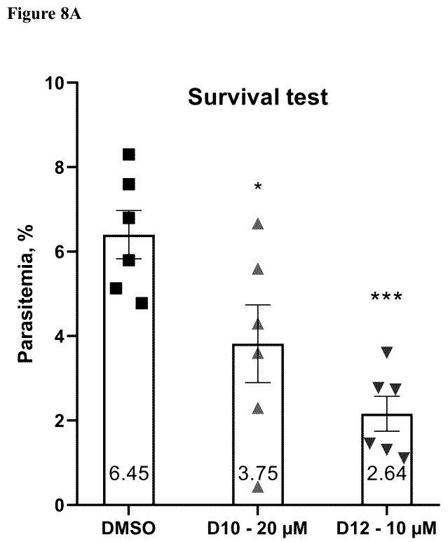

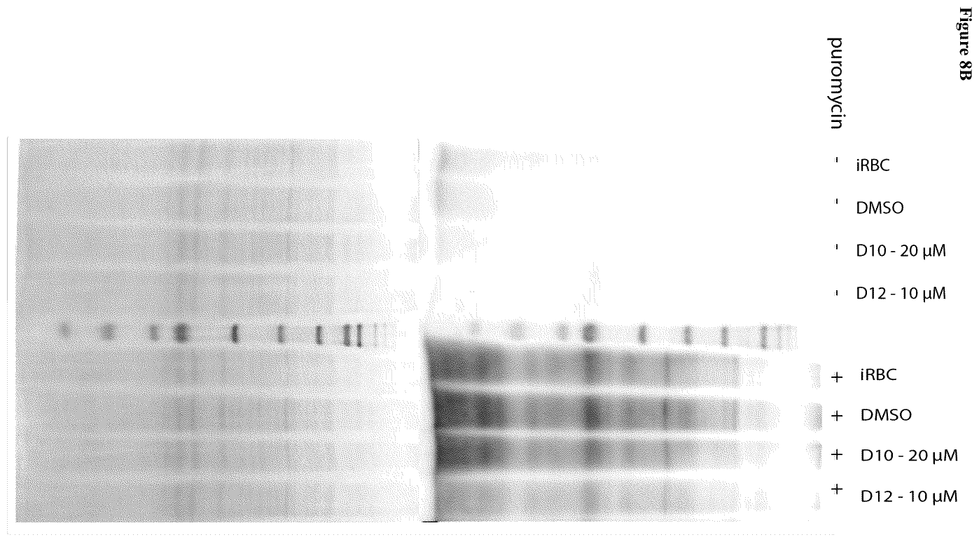

P. falciparum blood cultures were incubated with i14G1-10 (20 uM) or i14G1-12 (10 uM) for 1 hour, followed by a wash step with fresh medium. Parasitaemia levels were measured 24 hours later. Both inhibitors significantly reduced parasite levels in the blood, with i14G1-10 producing a 42% reduction and i14G1-12 producing an even better 69% reduction (FIG. 8A). Importantly, the human blood cells were not adversely affected by the inhibitors. This indicates that both molecules inhibit parasite growth/survival and are therapeutically effective treatments for killing the parasite without harming host cells.

Next, the method of action that caused the growth/survival inhibition was tested. P. falciparum blood cultures were incubated with i14G1-10 (20 uM) or i14G1-12 (10 uM) for 1 hour, as before, and then were either untreated or incubated with puromycin for 5 minutes. Puromycin labels newly synthesized proteins and cell lysates were analysed by western blot with anti-puromycin antibody (FIG. 8B). Both drugs inhibited protein synthesis and thereby puromycin incorporation, indicating that the drugs are effective in modulating P. falciparum translation. Once again i14G1-12 was superior to i14G1-10.

### Example 10: Inhibition of the eIF1-eIF4G1 Interaction Leads to 48S Instability and a Scanning Defect

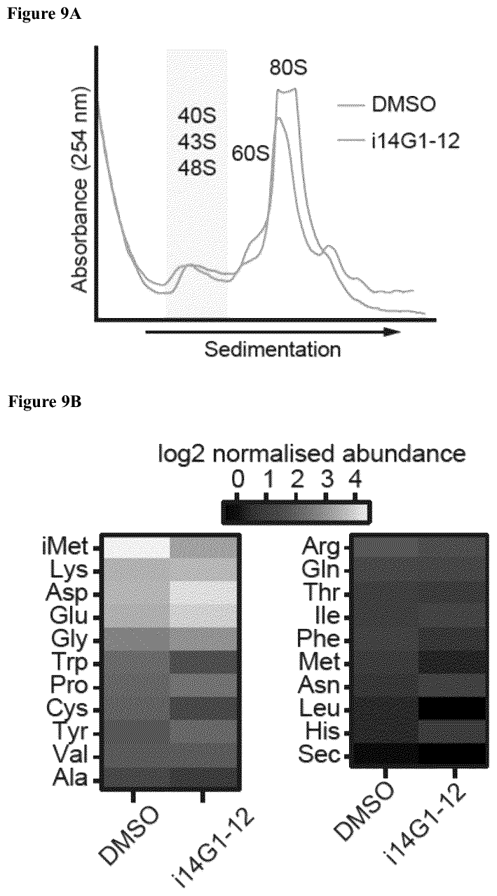

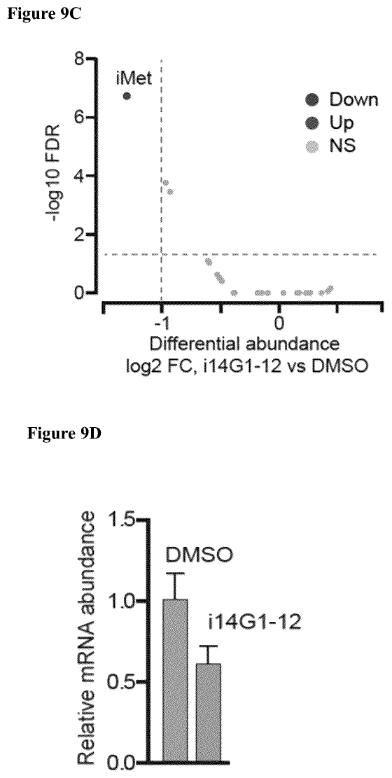

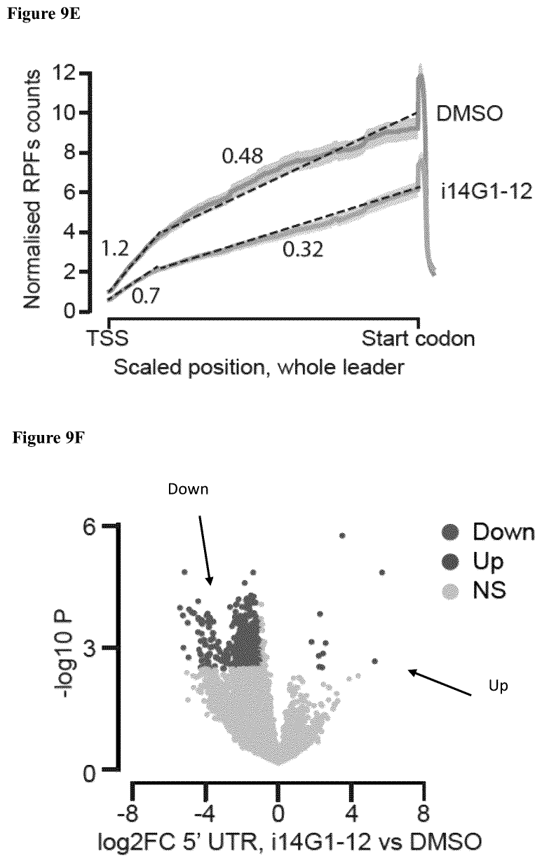

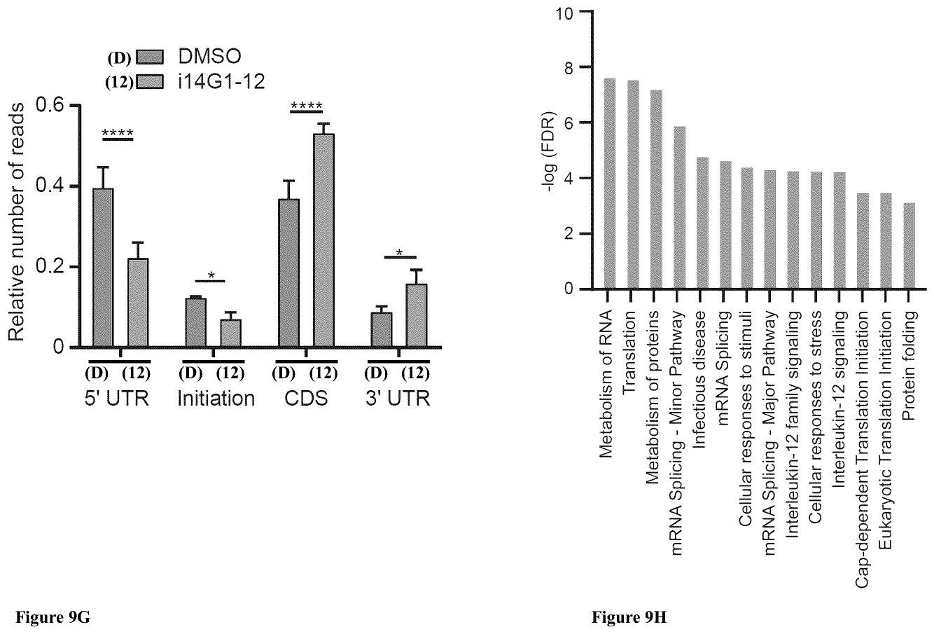

eIF1-eIF4G1 inhibitors of the invention, mimic the effects of eIF1 and eIF4G1 knockdown on translation initiation directed by very short and very long 5′UTRs and caused significant changes in translation determined by polysome and ribosome profiling. However, as eIF1-eIF4G1 interaction essentially exists during ribosome scanning and start codon recognition, the inventors were interested in analysing the role of these factors specifically at the scanning and initiation steps. For this purpose, the inventors set out to determine the footprints from 48S ribosomes using the translation complex profiling (TCP-seq) of cells treated with the eIF1-eIF4G1 inhibitor i14G1-12. As the 48S is less stable than the 80S, cells have to be crosslinked before lysis. Importantly, to maintain the integrity of the ribosome the inventors decreased the quantity of Rnase 1 by 100 fold. Three biological replicates of HEK293T cells treated with DMSO or with 5 μM of i14G1-12 for one hour followed by TCP-seq and RNA-seq for total RNA analysis were generated (FIG. 9A). The reproducibility of the replicates from the TCP-seq libraries was high. The RNA-seq libraries showed little changes between the control and the short-term i14G-12 treatment consistent with results mentioned hereinabove. The distribution of the length of the reads from the TCP-seq spanned from 17 nt to 80 nt with a vast majority being shorter than 40 nt. The iMet tRNA was found to be the most abundant tRNAs (relative to codon frequency) confirming the extraction of the 48S ribosomes (FIG. 9B). Interestingly, upon i14G1-12 inhibition, the iMet tRNA was the most downregulated tRNA (FIG. 9B-C). Moreover, the number of reads mapping to mRNAs was significantly decreased by i14G1-12 treatment in the three replicates relative to DMSO (FIG. 9D).

The distribution of the reads on the mRNA was assessed. As expected, most of the reads mapped to the 5′ UTR but reads on the CDS and 3′ UTR were also observed (FIG. 9G). Then the distribution of reads on the 5′ UTR using metagene analysis were specifically analysed (FIG. 9E). The reads accumulated towards the start codon, suggesting that the ribosome gains stability upon scanning or slows down due to queuing. The accumulation of reads from 5′ to 3′ occurs in two phases in which the first accumulates faster (FIG. 9E). The i14G1-12 caused a progressive reduction of the number of reads on the leader sequence as well as on the start codon relative to DMSO. The differentially scanned genes were analyzed and 476 downregulated genes (FIG. 9G) and only 10 upregulated genes were found. The differentially scanned mRNAs are mostly enriched with functions affecting mRNA fate from processing to translation (FIG. 9H). These observations support the concept that i14G1-12 treatment leads to destabilization of the scanning 48S and initiation defects.

### Example 11: Global Analysis of Leaky Scanning and the Critical Role of eIF4G1-eIF1 Interaction in its Restraining

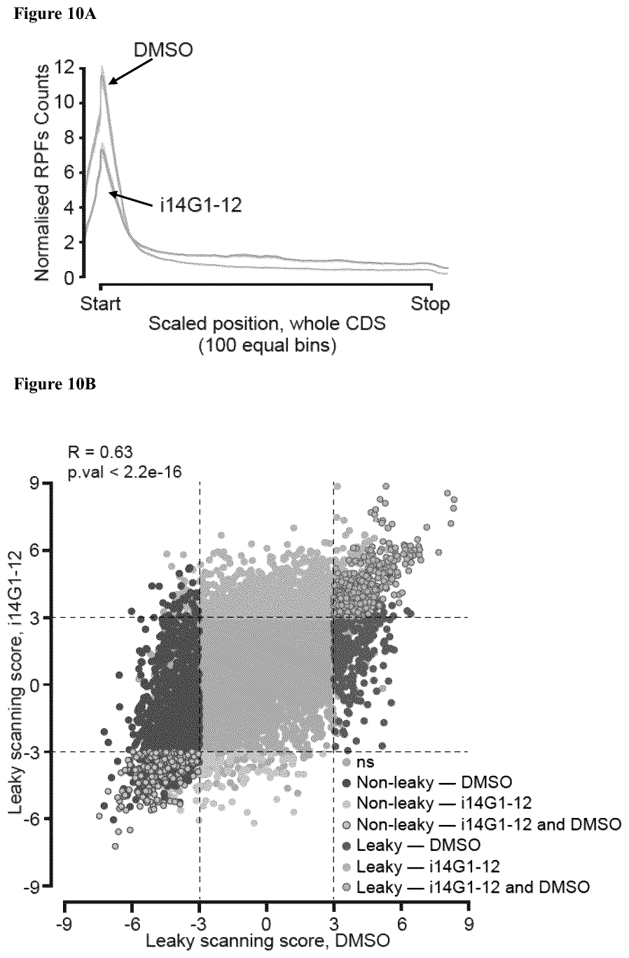

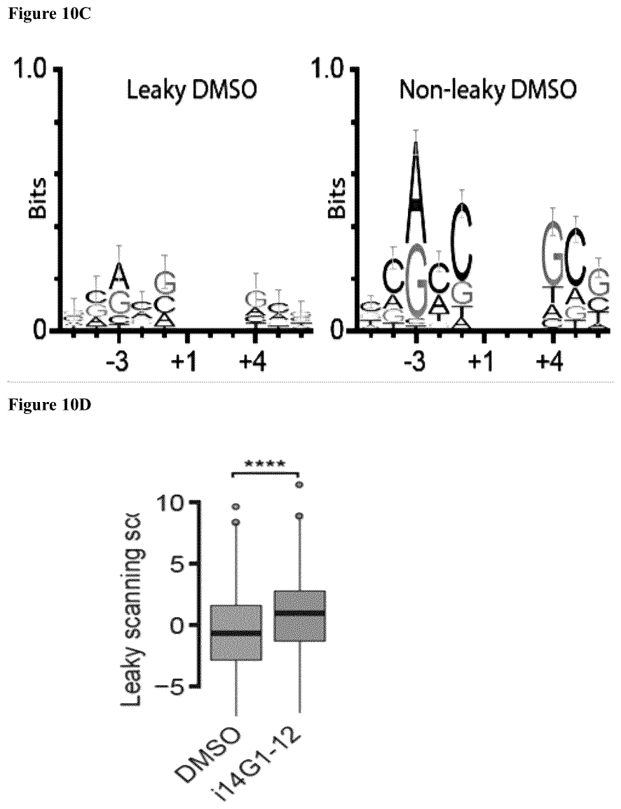

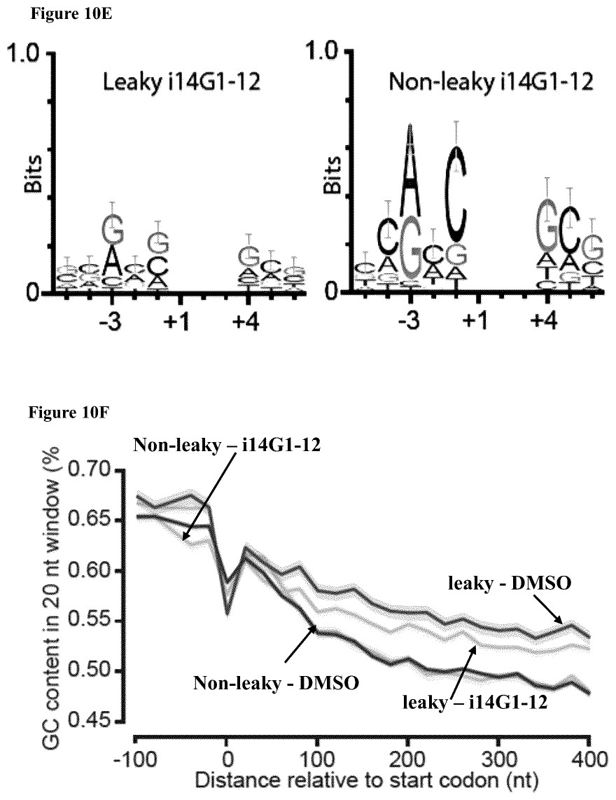

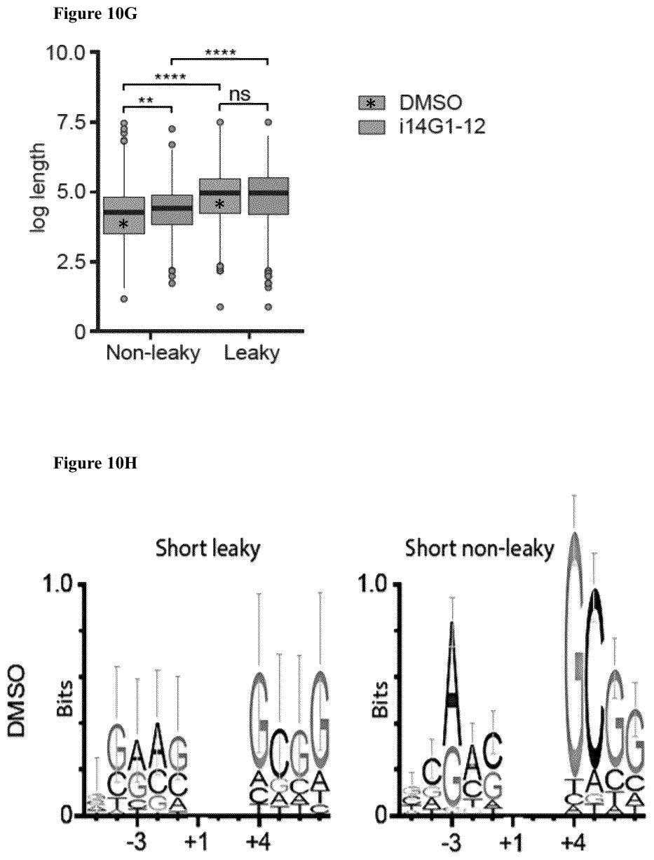

The data revealed that the reads did not exclusively map to the 5′ UTR but also to the CDS and the 3′ UTR (FIG. 9G). While reads mapping to the 3′ UTR likely come from readthrough, it was previously proposed that reads on the CDS originate from elongating ribosomes that dissociate during crosslinking, leaving the 40S subunit on the mRNA. On the other hand, the abundance of these reads was significantly increased by i14G1-12 (FIGS. 10A and 9G). Considering the central role of eIF1 and eIF4G1 in the AUG recognition, another possibility is that at least part of the reads mapping to the CDS originates from the ribosome bypassing the start codon. To test this hypothesis, a leaky scanning score (LS) for each mRNA was defined. The score was calculated by normalizing the RPKM number of footprints on the CDS with the number of footprints originating from initiating ribosomes. A positive score suggests the mRNA has more reads on the CDS than on the start codon. The inventors discriminated between two populations of mRNAs either with a strong negative score (LS<−3, defined as non-leaky) or with a strong positive score (LS >3, defined as leaky). In the DMSO condition, most of the genes had a significantly negative score (n=1223), but a population with a positive score (n=637) (FIG. 10B) was also identified. As the context predetermines if the translation machinery will bypass a start codon, the sequence around the annotated start codon of mRNAs with strong positive or strong negative scores was examined In accordance with the leaky scanning hypothesis, genes with negative scores had an overall stronger Kozak context than genes with positive scores (FIG. 10C, left vs. right panel). Interestingly, a C at position −1 is highly prevalent among the non-leaky genes. This result supports the hypothesis that the reads originating from the CDS region are not only the results of 80S ribosome dissociation but are primarily the consequence of leaky scanning. This is further supported by the observation that upon i14G1-12 treatment, there is a marked four-fold decrease in the number of non-leaky genes (n=325) and a more than two-fold increase in the number of leaky genes (n=1266, FIG. 10B). In line with the metagene analysis (FIG. 10A), the overall leaky score was increased by i14G1-12 (FIG. 10D). Regarding non-leaky genes, we observed that the enrichment of the cytosine at position −1 was further enhanced by i14G1-12 (FIG. 10C right panel vs. FIG. 10E right panel). The GC content of the non-leaky and leaky genes around the start codon was analysed, and non-leaky genes had a lower GC content than leaky genes (FIG. 10F). The difference was decreased upon i14G1-12 treatment suggesting lower GC content prevents leaky scanning. Regarding the length of mRNA features, non-leaky genes have significantly shorter leader sequence than leaky genes (FIG. 10G). On the other hand, non-leaky genes bear a significantly longer 5′ UTR in i14G1-12 than in DMSO (FIG. 10G), indicating for the relaxation of AUG recognition. GO terms analysis demonstrated that the non-leaky population was significantly enriched in genes involved in key functions such as translation and energy production (FIG. 10J). However, the leaky genes were significantly less enriched in specific pathways (FIG. 10J), suggesting that housekeeping genes are resistant to leaky scanning.

AUG codons placed proximal to the 5′ cap are known to be susceptible to leaky scanning. Thereof, the inventor took advantage of the leaky scanning data to examine how start codons positioned up to 30 nt from the 5′ cap perform. Surprisingly, a higher number of genes were non-leaky than leaky in DMSO (n=297, LS<0; n=81, LS >0). The context from non-leaky genes was highly enriched in purine at −3 and GCs at positions+4 to +7 (FIG. 10H right panel). The frequencies of the +4 G and +5 C were remarkably higher than those of non-leaky genes with longer leader sequences (FIG. 10C vs. 10H). The nucleotide enrichment of the short leader-non-leaky mRNA is reminiscent of the TISU element, of which positions+4, +5 and +6 were shown to prevent leaky scanning. Interestingly, leaky mRNAs with short leaders bore a stronger Kozak context than those with longer leaders, as they were enriched in +4 G (FIG. 10H). Upon eIF4G1-eIF1 inhibition, mRNAs displaying leaky scanning have a stronger Kozak context than under DMSO (FIG. 10I, n=99, LS<0; n=147, LS >0). However, mRNA with a TISU-like context remained resistant to inhibition. Thus, initiation from a short leader requires a more specific motif sequence than on longer leaders. Altogether, these results demonstrate that ribosomes on the CDS region are derived from ribosomes bypassing the start codon. Inhibition of the eIF4G1-eIF1 interaction impairs AUG recognition and promotes leaky scanning of mRNAs with weaker Kozak context and very short 5′UTR.

### Example 12: TCP-Seq can Distinguish AUG Recognition Modes

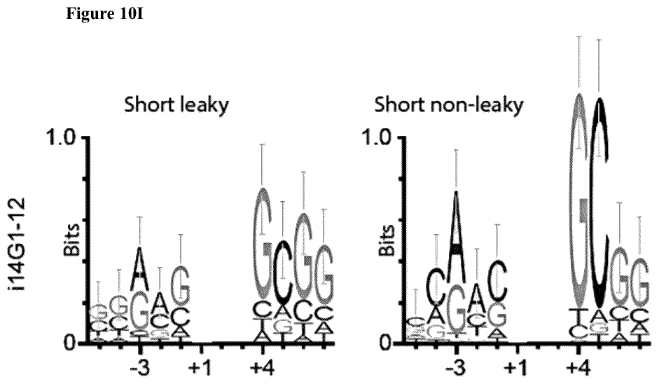

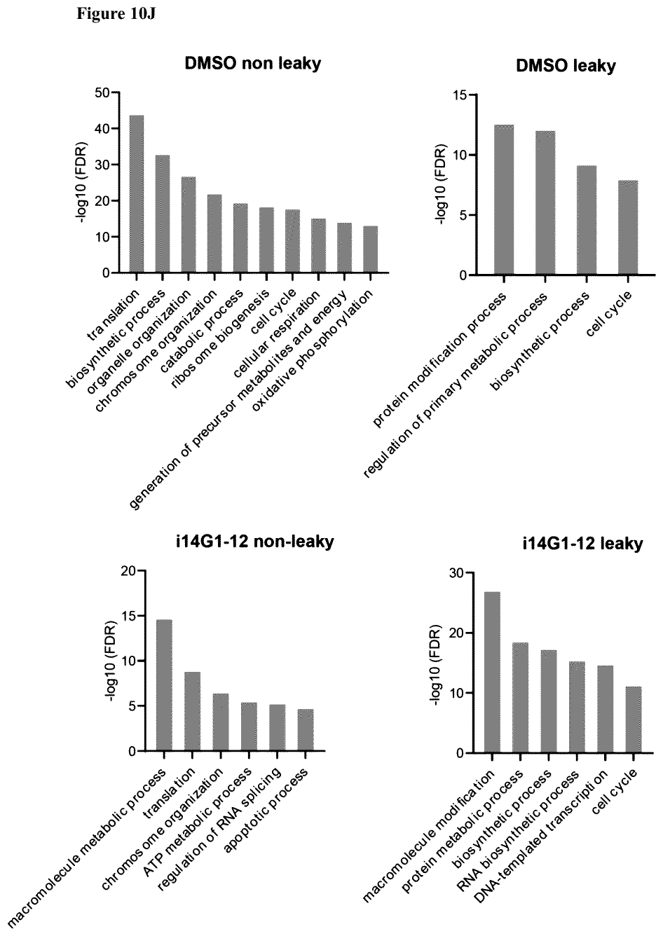

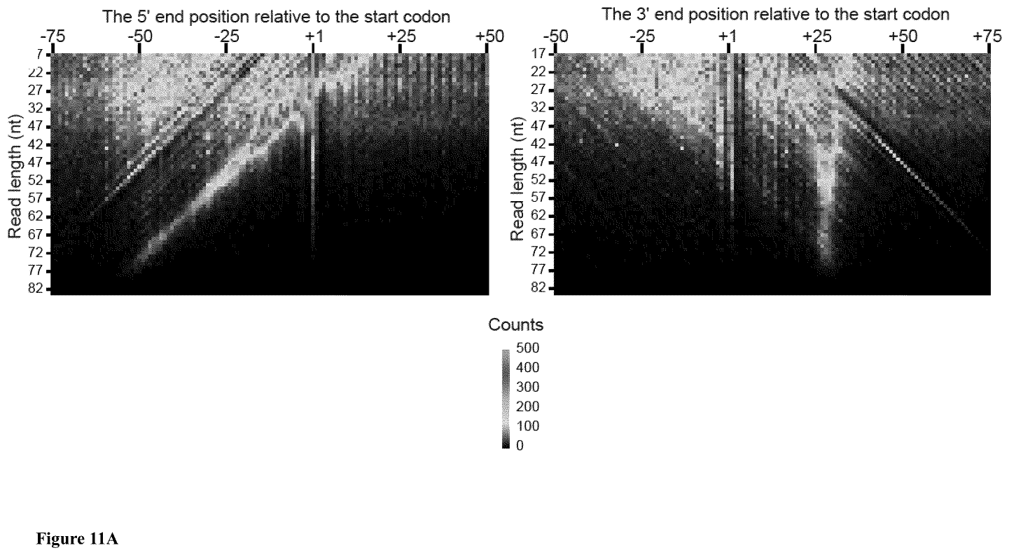

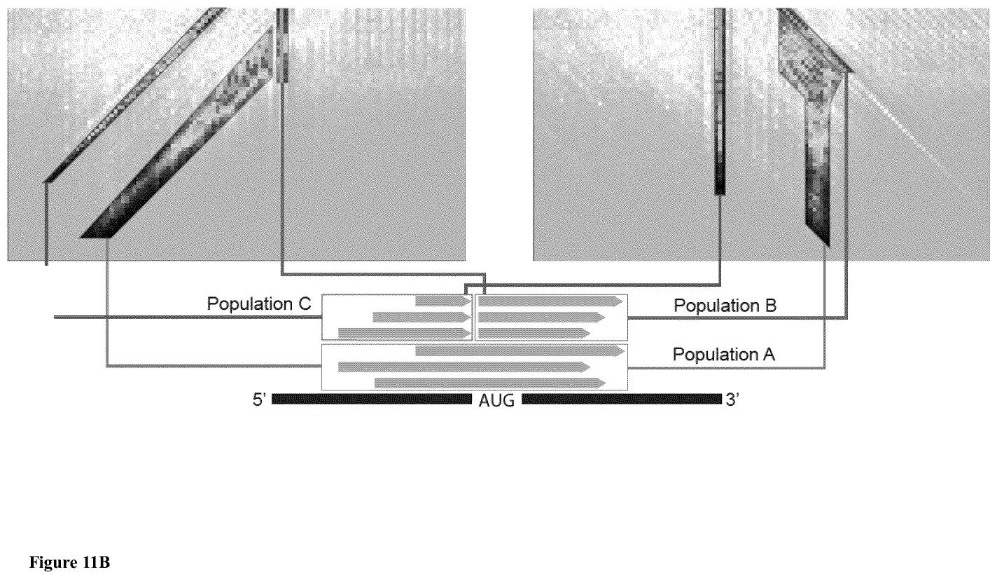

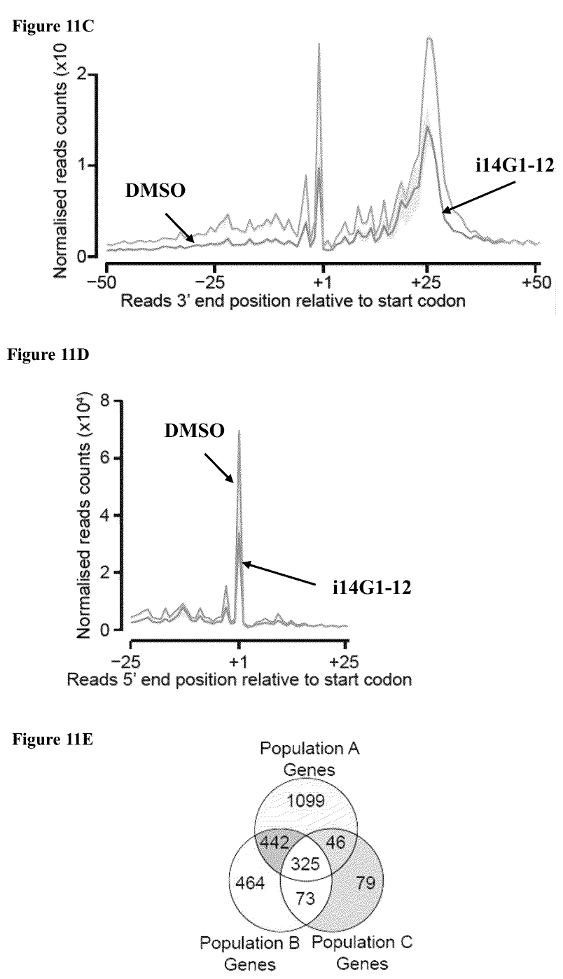

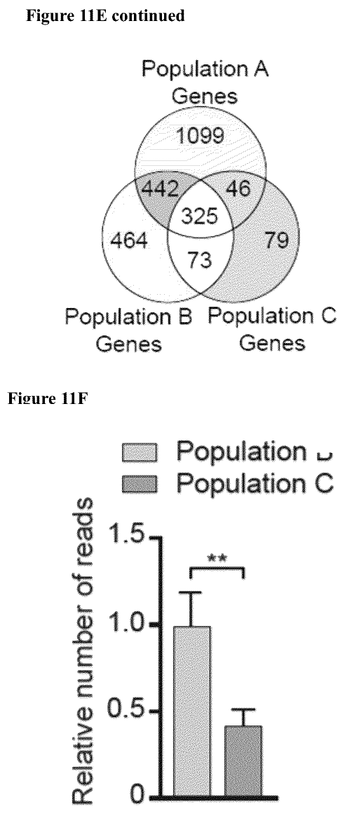

From the metagene analysis (FIGS. 9E and 10A), most of the reads accumulate at the start codon. The inventors assessed the distribution of the reads that span the AUG by their exact 5′ and 3′ boundaries and their length (FIG. 11A). The ends of the reads clustered at specific positions. In general, most of the 5′ ends are located upstream of the start codon at variable positions that depend on the read length (FIG. 11A, left panel). This observation suggests the 48S composition is heterogenous at the 5′ end. On the other hand, most of the 3′ ends were distributed in a narrow window of a few nucleotides downstream of the start codon (FIG. 11A, right panel). According to their length and ends positions, three populations of reads were defined namely A, B and C (FIG. 11B). Reads from population A were the most abundant and overlapped the start codon. The position of their 5′ end varied and their 3′ end is strictly located between +18 and +27 from the start codon and peaked at +24 to +27 (FIGS. 11A right panel and 11C). As they overlap the start codon, this population was defined as footprints from initiating ribosomes. Additionally, two other populations of reads (B and C) were distinguished that do not overlap the start codon. Population B consists of a relatively large number of footprints starting at +1 (FIGS. 11B and 11D) while population C footprints strictly end at −1 (FIGS. 11B and 11C). As in vitro data have shown that the P-site becomes accessible to the nuclease ReIE following AUG recognition, implying these reads are cleavage products from initiating ribosome footprints at −1/+1 by Rnase 1. In line with this notion, there is a significant overlap between populations B and C (FIG. 11E) suggesting that they originate from cleavage within the initiating 48S. According to the read counts, population B was more retained than population C suggesting that population C is less protected (FIG. 11F).

Treatment with i14G1-12 caused an overall diminution of the number of reads around the start codon consistent with the metagene plots (FIGS. 11C and 11D). However, the ratio of population B to A was significantly decreased by i14G1-12 suggesting a differential effect on initiation (FIG. 11F). To test this, the surrounding AUG sequence of genes that have at least one read from A, B and C in the three biological replicates were compared (FIG. 11G). All three populations bear a strong Kozak context as evident from the enrichment of A/G at position −3 and G at +4. However, a C at position −1 clearly distinguishes populations B and C genes from population A.

Based on the observation that initiation leads to cleavage at −1/+1, we searched for alternative upstream and internal open reading frames (uORF and iORF). The inventors examined clusters of 5′ end reads (similar to population B) and extracted the sequence of the 3 nt at the cluster site. Independently on the region, clusters on non-cognate AUG with an ANN base were discovered. The ACG and ATG codons were the most frequent codon in DMSO for uORF and iORFs, respectively. i14G1-12 treatment drastically decreased their frequency and of other non-cognate AUGs.

Altogether, these data demonstrate that initiating ribosomes from living cells can be observed in different modes which strongly depend on AUG context and start codon recognition by eIF1.

### Example 13: Differences in Start Codon Footprints Reveal the In Vivo Dynamics of the 48S Initiation Complex

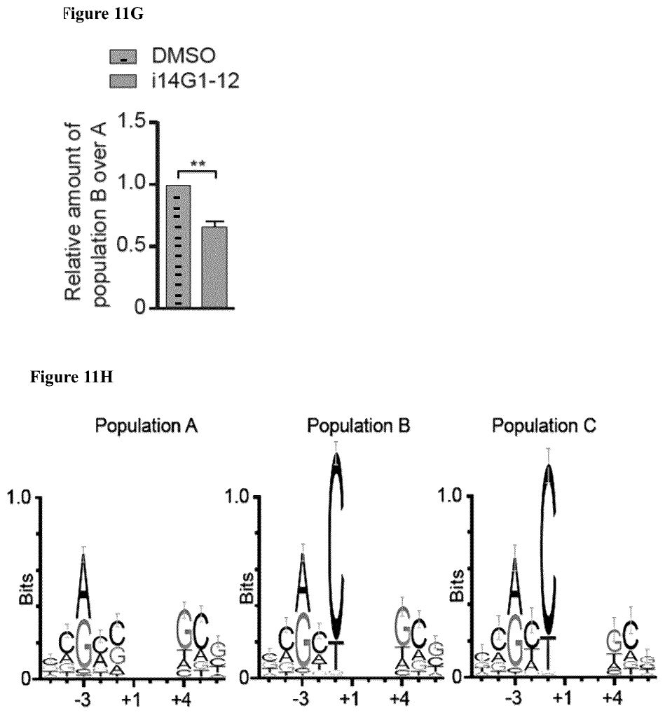

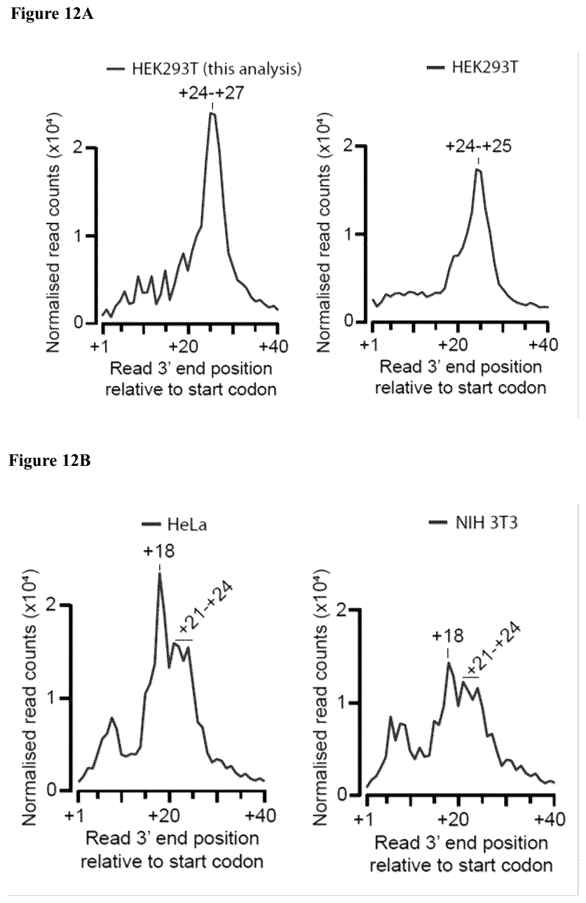

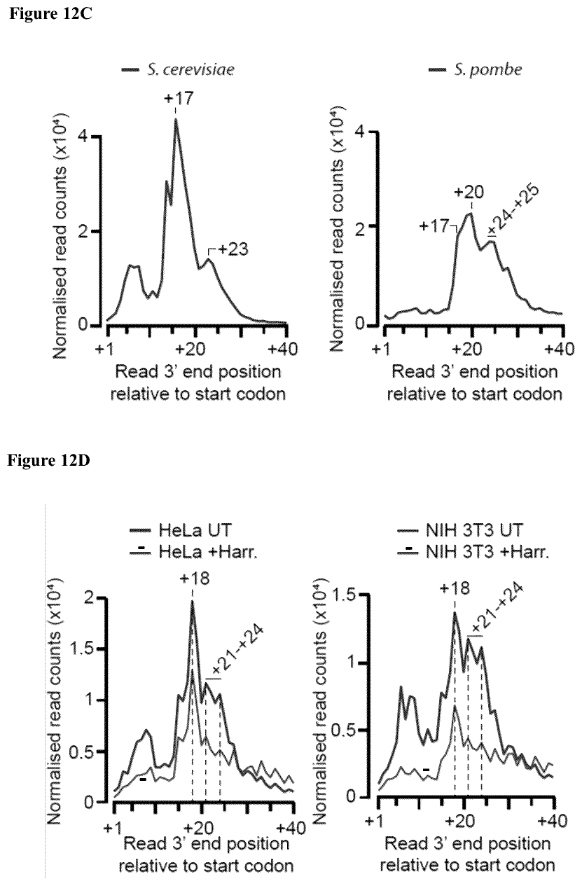

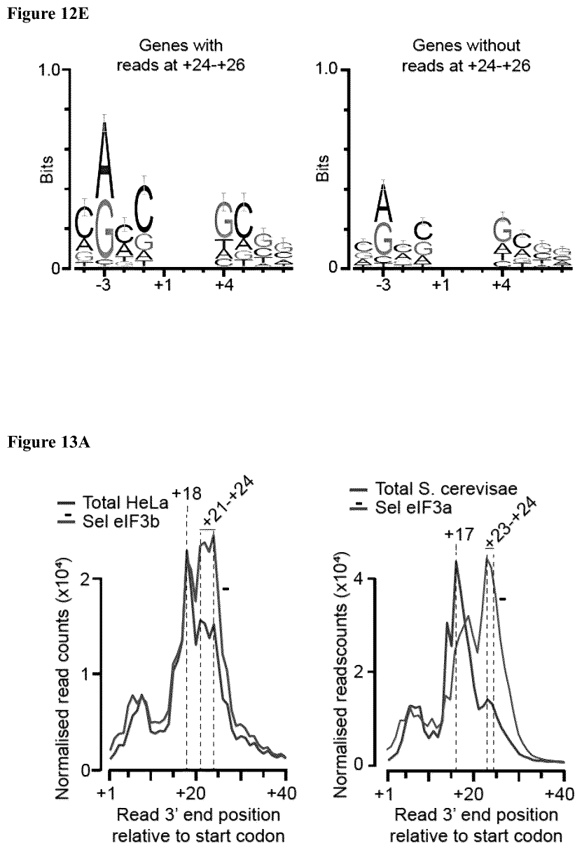

According to the distribution of the footprints around the start codon, most of the 3′ ends of populations A and B are positioned between +24 and +27 from the start codon (FIG. 11C). As these reads originate from initiating ribosomes, this finding suggests that the 48S covers the mRNA up to +27 from the P-site (FIG. 12A, left). However, in many previous in vitro assays such as toeprinting, the 48S and the 80S cover the mRNA up to +15 to +18 nt from the P site. To investigate this extension, the inventors analysed previously reported and publicly available TCP-seq data of mammalian and yeast cells. In samples of another reported TCP-seq from HEK293T cells, the 3′end of the footprints also extend downstream from to +24 and +25, (FIG. 12A, right). In HeLa cells and in the mouse fibroblast cell lines NIH3T3, a clear peak at +18 along with additional peaks from +21 to +24 were observed (FIG. 12B). The analysis of the TCP-seq data from yeast revealed a sharp peak at +17 in S. cerevisiae, while S. pombe showed both the +17 and the extended footprints at +20 to +25 (FIG. 12C). Interestingly, upon treatment with harringtonine, which is known to block ribosomes on start codons, the 3′ ends peaks from +21 to +24 shifted to +18 in HeLa and NIH 3T3 cells (FIG. 12D). The AUG initiation context of genes that have the +24 to +26 footprint were compared to those lacking it and found that the +24 to +26 extension is highly enriched in genes with a strong Kozak context (FIG. 12E).

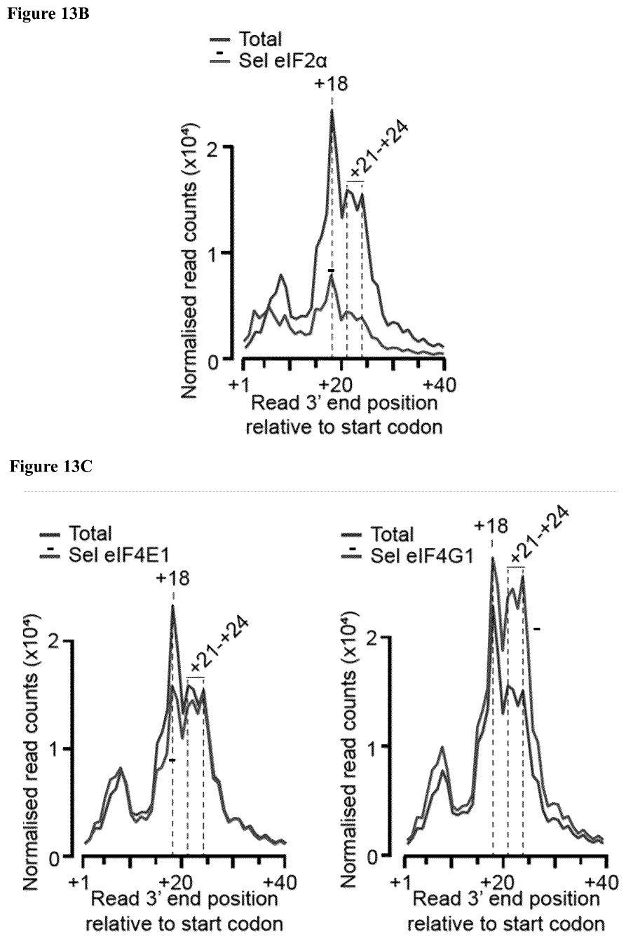

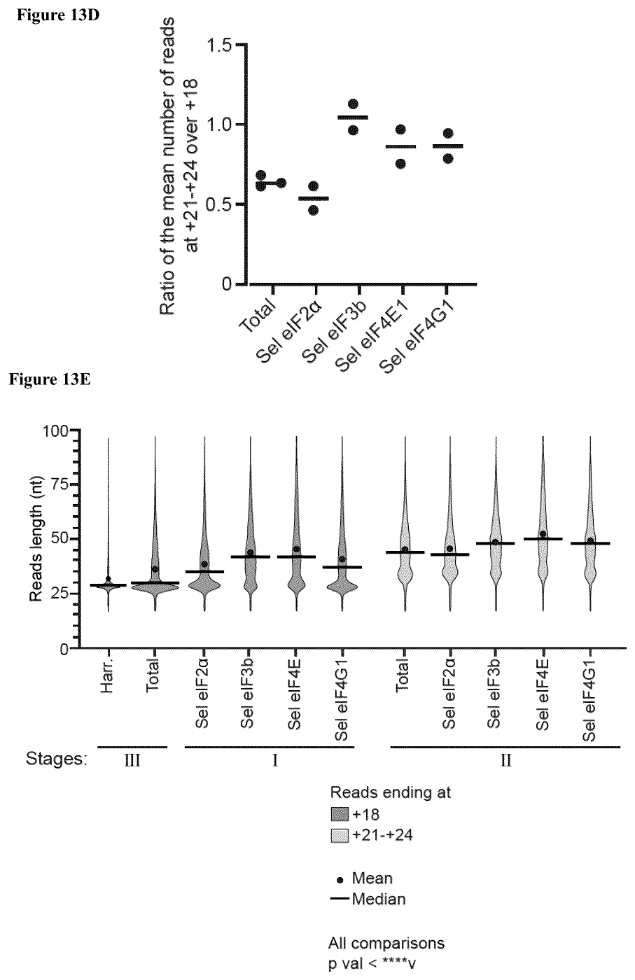

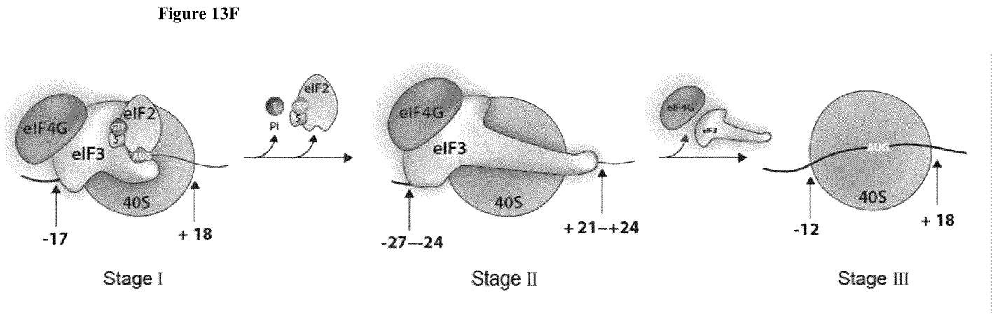

To better characterize the source of the footprint extension, publicly available selective TCP-seq data of HeLa and S. cerevisiae cells was analysed. In this procedure, the cross-linked 48S is immunoprecipitated with antibodies against a translation initiation factor and then subjected to TCP-seq. Remarkably, the extension at +21 to +25 is particularly enriched by the Sel-TCP-seq of eIF3 both in HeLa and yeast cells (FIG. 13A, 13D). On the other hand, the footprint extension is selectively depleted from the Sel-TCP-seq of eIF2α (FIG. 13B, 13D). The +21 to +24 extension are also enriched with the Sel-TCP-seq of eIF4E and eIF41G (FIG. 13C, 13D). The pattern of the latter confirms that the eIF4F members scan with the ribosome to the AUG. Altogether, these data reveal that eIF3 is responsible for the extension of the footprint of 48S ribosome after eIF2α release, providing in vivo evidence for ribosome conformation change upon start codon recognition.

To gain further insights on the initiation complex the length of the footprints ending either at +18 or the extended +21 to 24 was determined. While in the total sample and in treated cells with harringtonine, the median length of the +18 is 30, reflecting an upstream protection at −12 (FIG. 13F), in all the initiation factor Sel-TCP-seq the median is increased up to 42 nt (FIG. 13E, left violins). With the +21 to +24 footprints, the median length is 45 nt indicating that the 5′end is located at −21 to −24 (FIG. 13F). This extension is further increased with the Sel-TCP-seq of eIF4E, eIF4G1 and eIF3B but not eIF2α. Thus, the initiation factors extend the 5′-end protection of the initiation complex, indicating that the shorter footprints ending at −12 represent the late initiating small ribosomal subunit that released the eIFs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of inhibiting Eukaryotic translation initiation factor 4 gamma 1 (eIF4G1) binding to Eukaryotic translation initiation factor 1 (eIF1) comprising contacting said eIF4G1 with a compound, a salt thereof, a tautomer thereof, or any combination thereof; wherein said compound is represented by Formula II: 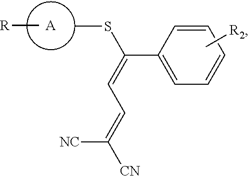
or by Formula I: 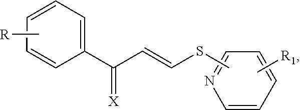
wherein:
each R and R₂ is independently selected from H, or is absent, or represents a substituent comprising any one of —NO₂, —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted;
each R′ is independently H or comprises an optionally substituted C₁-C₁₀ alkyl, an C₁-C₁₀ alkyl-aryl, an C₁-C₁₀ alkyl-cycloalkyl, optionally substituted C₃-C₁₀ cycloalkyl, optionally substituted C₃-C₁₀ heterocyclyl, optionally substituted heteroaryl, optionally substituted aryl or a combination thereof,
A represents any of cycloalkyl, aryl, and heteroaryl, a fused aryl, a fused cycloalkyl or any combination thereof,
X comprises O, S, NH, or NR′;
R₁ is H or represents a substituent comprising any one of an electron withdrawing group; —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted.

2. The method of claim 1, wherein said R₁ is said electron withdrawing group.

3. The method of claim 1, wherein said X is O.

4. The method of claim 1, wherein said A is heteroaryl, and wherein said R₂ is not H.

5. The method of claim 1, wherein said compound is selected from:

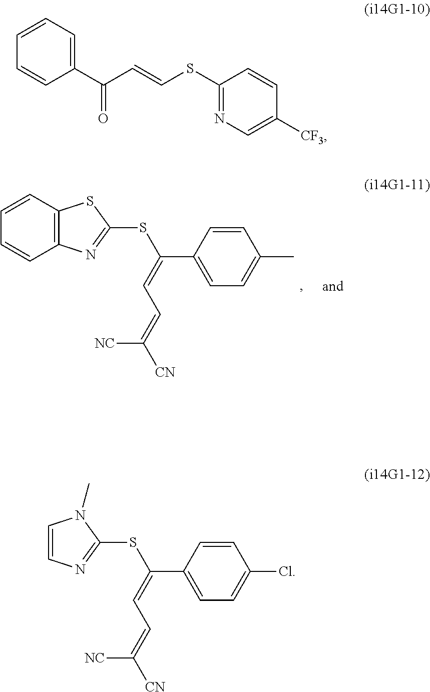 [SMILES: O=C(/C=C/Sc1ccc(C(F)(F)F)cn1)c1ccccc1 ; Cc1ccc(/C(=C\C=C(C#N)C#N)Sc2nc3ccccc3s2)cc1 ; Cn1ccnc1S/C(=C/C=C(C#N)C#N)c1ccc(Cl)cc1]

6. The method of claim 1, wherein said eIF4G1 and said eIF1 are in a cell and said method is a method of decreasing translation initiation in said cell.

7. The method of claim 6, wherein said contacting is with i14G1-10 and wherein said contacting further increases binding of said eIF4G1 to Eukaryotic translation initiation factor 4E (eIF4E).

8. The method of claim 6, wherein said method is a method of increasing translation from open reading frames with short 5′ UTRs and decreasing translation from open reading frames with long 5′ UTRs.

9. (canceled)

10. The method of claim 9, wherein at least one of: said method is a method of increasing recognition of non-AUG translational start codons; said method is a method of increasing recognition of a most cap-proximal AUG codon and decreasing leaky recognition of more downstream AUG codons: said method is a method of increasing translation of at least one stress-response protein: said method is a method of killing said cell.

11. (canceled)

12. (canceled)

13. (canceled)

14. The method of claim 10, wherein said stress-response protein is selected from an unfolded protein response (UPR) pathway protein, an endoplasmic reticulum (ER)-stress response pathway protein and a UV-response pathway protein, Activating transcription factor 3 (ATF3), Activating transcription factor 4 (ATF4), Growth arrest and DNA damage inducible alpha (GADD45A), DNA damage inducible transcript 3 (DDIT3) and Protein phosphatase 1 regulatory subunit 15A (PPP1R15A or GADD34).

15. (canceled)

16. (canceled)

17. The method of claim 10, wherein said cell is characterized by increased protein expression or increased number of upstream open reading frames (uORFs) as compared to a healthy control cell.

18. The method of claim 1, wherein said contacting is with either i14G1-10, i14G1-12 or a combination thereof.

19. A method of reducing translation in a target cell, the method comprising reducing binding of eIF4G1 to eIF1 without increasing binding of eIF4G1 to eIF4E in said cell, thereby reducing translation in a target cell.

20. The method of claim 19, comprising contacting said cell with a compound that binds eIF4G1 at an eIF1 binding site and occludes, blocks or otherwise makes inaccessible an eIF4E binding site in eIF4G1.

21. The method of claim 20, wherein said compound is represented by Formula II or a salt, tautomer, or functional derivative thereof.

22. The method of claim 20, wherein said agent is i14G1-11, i14G-12.

23. A pharmaceutical composition comprising a compound, a salt thereof, a tautomer thereof, a functional derivative thereof or any combination thereof; wherein said compound is represented by Formula II: 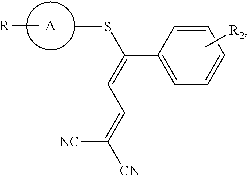
or by Formula I: 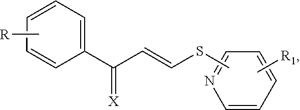
wherein:
each R and R₂ is independently selected from H, or is absent, or represents a substituent comprising any one of —NO₂, —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted;
each R′ is independently H or comprises an optionally substituted C₁-C₁₀ alkyl, an C₁-C₁₀ alkyl-aryl, an C₁-C₁₀ alkyl-cycloalkyl, optionally substituted C₃-C₁₀ cycloalkyl, optionally substituted C₃-C₁₀ heterocyclyl, optionally substituted heteroaryl, optionally substituted aryl or a combination thereof,
A represents any of cycloalkyl, aryl, and heteroaryl, a fused aryl, a fused cycloalkyl or any combination thereof,
X comprises O, S, NH, or NR′;
R₁ is H or represents a substituent comprising any one of: an electron withdrawing group; —CN, —OH, —CONH₂, —CONR′₂, —CNNR′₂, —CSNR′₂, —CONH—OH, —CONH—NH₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, carbonyl, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted.
and pharmaceutically acceptable carrier, excipient or adjuvant; and wherein said functional derivative is functional in inhibiting interaction of eiF4G1 and eIF1.

24. The pharmaceutical composition of claim 23, wherein said R₁ is said electron withdrawing group: wherein said X is O: wherein said A is heteroaryl and wherein said R2 is not H.

25.-36. (canceled)


SEQUENCE LISTING (51 sequences)

SEQ ID NO: 1 - DNA, 20 bp, synthetic construct
  misc_feature 1..20: note = Synthetic
  source 1..20: mol_type = other DNA
gcatcgtaggcaccgtatcc

SEQ ID NO: 2 - DNA, 34 bp, synthetic construct
  misc_feature 1..34: note = Synthetic
  source 1..34: mol_type = other DNA
agatcggaagagcacacgtctgaactccagtcac

SEQ ID NO: 3 - skipped

SEQ ID NO: 4 - DNA, 46 bp, synthetic construct
  misc_feature 1..46: note = Synthetic
  source 1..46: mol_type = other DNA
aacgcaataatacgactcactatagggctctgagctattccagaag

SEQ ID NO: 5 - DNA, 43 bp, synthetic construct
  misc_feature 1..43: note = Synthetic
  source 1..43: mol_type = other DNA
tttttttttttttttttttttttacaatttggactttccgccc

SEQ ID NO: 6 - DNA, 21 bp, synthetic construct
  misc_feature 1..21: note = Synthetic
  source 1..21: mol_type = other DNA
aggtcggtgtgaacggatttg

SEQ ID NO: 7 - DNA, 19 bp, synthetic construct
  misc_feature 1..19: note = Synthetic
  source 1..19: mol_type = other DNA
ggggtcgttgatggcaaca

SEQ ID NO: 8 - DNA, 22 bp, synthetic construct
  misc_feature 1..22: note = Synthetic
  source 1..22: mol_type = other DNA
caccattggcaatgagcggttc

SEQ ID NO: 9 - DNA, 22 bp, synthetic construct
  misc_feature 1..22: note = Synthetic
  source 1..22: mol_type = other DNA
aggtctttgcggatgtccacgt

SEQ ID NO: 10 - DNA, 21 bp, synthetic construct
  misc_feature 1..21: note = Synthetic
  source 1..21: mol_type = other DNA
ggaggatagagcctcacatgc

SEQ ID NO: 11 - DNA, 20 bp, synthetic construct
  misc_feature 1..20: note = Synthetic
  source 1..20: mol_type = other DNA
tgttcgaggatctgtgccaa

SEQ ID NO: 12 - DNA, 20 bp, synthetic construct
  misc_feature 1..20: note = Synthetic
  source 1..20: mol_type = other DNA
gtgctctatggaggcatcgc

SEQ ID NO: 13 - DNA, 20 bp, synthetic construct
  misc_feature 1..20: note = Synthetic
  source 1..20: mol_type = other DNA
tgaggcttgtgtctcggatt

SEQ ID NO: 14 - DNA, 20 bp, synthetic construct
  misc_feature 1..20: note = Synthetic
  source 1..20: mol_type = other DNA
cttggactcacagaccggag

SEQ ID NO: 15 - DNA, 19 bp, synthetic construct
  misc_feature 1..19: note = Synthetic
  source 1..19: mol_type = other DNA
ggcagggtaggagtacagc

SEQ ID NO: 16 - DNA, 20 bp, synthetic construct
  misc_feature 1..20: note = Synthetic
  source 1..20: mol_type = other DNA
acctgcaaggaagaagcgat

SEQ ID NO: 17 - DNA, 21 bp, synthetic construct
  misc_feature 1..21: note = Synthetic
  source 1..21: mol_type = other DNA
catcaacgtaccagtttcgcc

SEQ ID NO: 18 - DNA, 20 bp, synthetic construct
  misc_feature 1..20: note = Synthetic
  source 1..20: mol_type = other DNA
gagactcgggtgatagcgtg

SEQ ID NO: 19 - DNA, 20 bp, synthetic construct
  misc_feature 1..20: note = Synthetic
  source 1..20: mol_type = other DNA
gtggtgctgtctgaactgga

SEQ ID NO: 20 - DNA, 20 bp, synthetic construct
  misc_feature 1..20: note = Synthetic
  source 1..20: mol_type = other DNA
cagcctcgcctcacatacaa

SEQ ID NO: 21 - DNA, 20 bp, synthetic construct
  misc_feature 1..20: note = Synthetic
  source 1..20: mol_type = other DNA
gtgccatccggtaaggtcaa

SEQ ID NO: 22 - DNA, 20 bp, synthetic construct
  misc_feature 1..20: note = Synthetic
  source 1..20: mol_type = other DNA
cccacaaaccacctggacat

SEQ ID NO: 23 - DNA, 20 bp, synthetic construct
  misc_feature 1..20: note = Synthetic
  source 1..20: mol_type = other DNA
ttcgcatacccacaactccc

SEQ ID NO: 24 - DNA, 22 bp, synthetic construct
  misc_feature 1..22: note = Synthetic
  source 1..22: mol_type = other DNA
cccttcaccttcttacaacctc

SEQ ID NO: 25 - DNA, 22 bp, synthetic construct
  misc_feature 1..22: note = Synthetic
  source 1..22: mol_type = other DNA
tgcccagctctaaactaaagga

SEQ ID NO: 26 - DNA, 20 bp, synthetic construct
  misc_feature 1..20: note = Synthetic
  source 1..20: mol_type = other DNA
cctctgcgctggaatcagtc

SEQ ID NO: 27 - DNA, 22 bp, synthetic construct
  misc_feature 1..22: note = Synthetic
  source 1..22: mol_type = other DNA
ttctttctcgtcgcctcttttt

SEQ ID NO: 28 - DNA, 20 bp, synthetic construct
  misc_feature 1..20: note = Synthetic
  source 1..20: mol_type = other DNA
gaacggctcaagcagaaatc

SEQ ID NO: 29 - DNA, 22 bp, synthetic construct
  misc_feature 1..22: note = Synthetic
  source 1..22: mol_type = other DNA
ttcaccattcggtcaatcagag

SEQ ID NO: 30 - DNA, 22 bp, synthetic construct
  misc_feature 1..22: note = Synthetic
  source 1..22: mol_type = other DNA
aaccacggaggataaaagaaca

SEQ ID NO: 31 - DNA, 21 bp, synthetic construct
  misc_feature 1..21: note = Synthetic
  source 1..21: mol_type = other DNA
ctgaacgatactcccaggacc

SEQ ID NO: 32 - DNA, 23 bp, synthetic construct
  misc_feature 1..23: note = Synthetic
  source 1..23: mol_type = other DNA
gccgctaaacttgcatatcttca

SEQ ID NO: 33 - DNA, 23 bp, synthetic construct
  misc_feature 1..23: note = Synthetic
  source 1..23: mol_type = other DNA
tcacacgtagtagcaaaagaacc

SEQ ID NO: 34 - DNA, 23 bp, synthetic construct
  misc_feature 1..23: note = Synthetic
  source 1..23: mol_type = other DNA
cgacaacaagaagactcgcatca

SEQ ID NO: 35 - DNA, 22 bp, synthetic construct
  misc_feature 1..22: note = Synthetic
  source 1..22: mol_type = other DNA
tgtgcgatggtgactttgccca

SEQ ID NO: 36 - DNA, 22 bp, synthetic construct
  misc_feature 1..22: note = Synthetic
  source 1..22: mol_type = other DNA
agccgcaaggagagctattcag

SEQ ID NO: 37 - DNA, 22 bp, synthetic construct
  misc_feature 1..22: note = Synthetic
  source 1..22: mol_type = other DNA
cgctcgaagatgtcgttgacga

SEQ ID NO: 38 - DNA, 22 bp, synthetic construct
  misc_feature 1..22: note = Synthetic
  source 1..22: mol_type = other DNA
gaaggatggcaagaagcgcaag

SEQ ID NO: 39 - DNA, 22 bp, synthetic construct
  misc_feature 1..22: note = Synthetic
  source 1..22: mol_type = other DNA
caaggtgctgaagcaagtccac

SEQ ID NO: 40 - DNA, 22 bp, synthetic construct
  misc_feature 1..22: note = Synthetic
  source 1..22: mol_type = other DNA
aggtgatggtcgagcgcttgtt

SEQ ID NO: 41 - DNA, 19 bp, synthetic construct
  misc_feature 1..19: note = Synthetic
  source 1..19: mol_type = other DNA
aacaagcgctcgaccatca

SEQ ID NO: 42 - DNA, 19 bp, synthetic construct
  misc_feature 1..19: note = Synthetic
  source 1..19: mol_type = other DNA
cctttggggttgggcttta

SEQ ID NO: 43 - DNA, 21 bp, synthetic construct
  misc_feature 1..21: note = Synthetic
  source 1..21: mol_type = other DNA
taggccaccacggccggatcc

SEQ ID NO: 44 - DNA, 23 bp, synthetic construct
  misc_feature 1..23: note = Synthetic
  source 1..23: mol_type = other DNA
ctgcagcttaagttcgagactgt

SEQ ID NO: 45 - DNA, 22 bp, synthetic construct
  misc_feature 1..22: note = Synthetic
  source 1..22: mol_type = other DNA
tggtggccggacaacccttagc

SEQ ID NO: 46 - DNA, 39 bp, synthetic construct
  misc_feature 1..39: note = Synthetic
  source 1..39: mol_type = other DNA
cggatctcatgagaagcgattcaaagtactagtagctgg

SEQ ID NO: 47 - DNA, 31 bp, synthetic construct
  misc_feature 1..31: note = Synthetic
  source 1..31: mol_type = other DNA
gcttctcatgagatccggctgctaacaaagc

SEQ ID NO: 48 - DNA, 28 bp, synthetic construct
  misc_feature 1..28: note = Synthetic
  source 1..28: mol_type = other DNA
gttgtccggccaccagtctgatgaagca

SEQ ID NO: 49-50 - skipped (2 entries)

SEQ ID NO: 51 - DNA, 11 bp, synthetic construct
  misc_feature 1..11: note = Synthetic
  source 1..11: mol_type = other DNA
atcgtatgtcc